SEQUENCE LISTING (20 sequences)

SEQ ID NO: 1 - DNA, 20 bp, Artificial Sequence
  Synthesized
tttggaagacatgagctcga

SEQ ID NO: 2 - DNA, 20 bp, Artificial Sequence
  Synthesized
aagggctgaaccaactcaga

SEQ ID NO: 3 - DNA, 20 bp, Artificial Sequence
  Synthesized
gactgtccgagcttgtgaaa

SEQ ID NO: 4 - DNA, 20 bp, Artificial Sequence
  Synthesized
ggccgtccatgtgaatgtat

SEQ ID NO: 5 - DNA, 21 bp, Artificial Sequence
  Synthesized
tgaaagaagactcacggagga

SEQ ID NO: 6 - DNA, 20 bp, Artificial Sequence
  Synthesized
tagcaaacgctccaaatggt

SEQ ID NO: 7 - DNA, 20 bp, Artificial Sequence
  Synthesized
ttgatgatttggggagaagg

SEQ ID NO: 8 - DNA, 20 bp, Artificial Sequence
  Synthesized
tggatgaaggacgtgtctca

SEQ ID NO: 9 - DNA, 20 bp, Artificial Sequence
  Synthesized
ggtggctatgcagagaaagc

SEQ ID NO: 10 - DNA, 20 bp, Artificial Sequence
  Synthesized
cagggaagaccacagctgag

SEQ ID NO: 11 - DNA, 20 bp, Artificial Sequence
  Synthesized
agctgagggacaacaacacc

SEQ ID NO: 12 - DNA, 20 bp, Artificial Sequence
  Synthesized
ggccgtccatgtgaatgtat

SEQ ID NO: 13 - DNA, 20 bp, Artificial Sequence
  Synthesized
tgaaagcagcaggaataggc

SEQ ID NO: 14 - DNA, 20 bp, Artificial Sequence
  Synthesized
caggcgcctcttcttagttg

SEQ ID NO: 15 - DNA, 22 bp, Artificial Sequence
  Synthesized
ctggagttttcttgtgcagttg

SEQ ID NO: 16 - DNA, 20 bp, Artificial Sequence
  Synthesized
aaacccaagcttcatgcaag

SEQ ID NO: 17 - DNA, 20 bp, Artificial Sequence
  Synthesized
gggtataaagcattgccagg

SEQ ID NO: 18 - DNA, 21 bp, Artificial Sequence
  Synthesized
gccttcaatgagccaagtaca

SEQ ID NO: 19 - DNA, 20 bp, Artificial Sequence
  Synthesized
ctttggccaagacaatgtca

SEQ ID NO: 20 - DNA, 20 bp, Artificial Sequence
  Synthesized
gtgtgagatgcagtgcccta

(19) United States
(12) Patent Application Publication
(10) Pub. No.: US 2023/0143011 A1
(43) Pub. Date: May 11, 2023

(54) METHODS OF DETECTING circRNA

(71) Applicant: Washington University, St. Louis, MO (US)

(72) Inventors: CARLOS CRUCHAGA, St. Louis, MO (US); LAURA IBANEZ, St. Louis, MO (US); UMBER DUBE, St. Louis, MO (US)

(21) Appl. No.: 17/766,112

(22) PCT Filed: Oct. 2, 2020
(86) PCT No.: PCT/US2020/054123

§ 371 Date: Apr. 1, 2022

Related U.S. Application Data
(60) Provisional application No. 62/909,316, filed on Oct. 2, 2019.

(51) Int. Cl. G01N 33/68; C12Q 1/6883

(52) U.S. Cl. CPC G01N 33/6896; C12Q 1/6883; G01N 2800/2821

(57) ABSTRACT

The present disclosure relates to a method to detect or measure circRNAs in a biological sample of a subject. In particular the present disclosure is directed to a method for diagnosing a neurological, neurodegenerative, or neuropathological disease such as Alzheimer's disease, in a subject and comprises the steps of—determining the level of one or more circRNA in a sample of a biological sample of said subject.

### CROSS-REFERNCE TO RELATED APPLICATION

This application claims priority from U.S. Provisional Application No. 62/909,316 filed on Oct. 2, 2019, which is incorporated herein by reference in its entirety.

### FIELD OF THE TECHNOLOGY

This present disclosure generally relates to methods for detection of circular RNA in a biological sample of a subject.

### REFERENCE TO SEQUENCE LISTING

This application contains a Sequence Listing that has been submitted in ASCII format via EFS-Web and is hereby incorporated by reference in its entirety. The ASCII copy, created on Sep. 29, 2020, is named 667595_ST25.txt, and is 3.86 KB bytes in size.

### BACKGROUND

Circular RNAs (circRNAs) are a class of RNAs that result from backsplicing events, in which the 3′ ends of transcripts are covalently spliced with the 5′ ends thereby forming continuous loops. As RNA sequencing has become widespread, thousands of circRNAs have been identified across eukaryotes. These studies have found circRNAs to be are highly expressed in the nervous system and enriched in synapses. In the brain, circRNA expression can occur independently of linear transcript expression, and may be a gene's most highly expressed isoform. Brain circRNAs are also regulated during development and in response to neuronal excitation. CircRNAs accumulate in aging mouse and fly brains, possibly due to their lack of free hydroxyl ends conferring resistance to exonucleases. Much is still unknown regarding circRNA biology; for example, it was only recently demonstrated that circRNAs can be translated in vivo. Thus far, the most well-established role of circRNAs is in microRNA (miRNA) regulation via sequestration leading to loss of function.

Alzheimer disease (AD) is a progressive, neurodegenerative disorder and the most common cause of dementia, affecting millions worldwide. AD is neuropathologically characterized by the accumulation of amyloid beta plaques and tau inclusions as well as widespread neuronal atrophy which results in dramatic cognitive impairment. Unfortunately, no effective preventative, palliative, or curative therapies currently exist for AD.

Therefore, a need in the art exists for a rapid practical and reliable test to guide physicians and patients in the decision-making process during treatment and care of neurodegenerative disorders.

### SUMMARY

Among the various aspects of the present disclosure is the provision of methods to detect or measure circRNAs in a biological sample or biological fluid of a subject. Briefly, therefore, the present disclosure is directed to detecting circRNAs associated with neurological, neurodegenerative, or neuropathological diseases in the biological fluid of a subject.

An aspect of the present disclosure provides for a method of measuring differential expression of circular RNA (circRNA DE). In some embodiments, the method comprises providing or having been provided a biological sample; extracting circRNA from the biological sample; detecting circRNA using a transcriptome-wide analysis; and/or determining the measured differential expression (DE) of the circRNA. In some embodiments, the transcriptome-wide analysis comprises quantifying specific transcript levels and analyses, wherein the analyses comprises a series of custom algorithms and methods, to predict a disease status.

In some embodiments, the method comprises identifying backsplice junctions using a calling and filtering approach; and/or collapsing the backsplice junctions onto the backsplice junctions annotated linear gene of origin or cognate linear mRNA.

In some embodiments, the method comprises excluding backsplices without a linear gene of origin annotation. In some embodiments, the method comprises determining expression levels of circRNA in the biological sample; and/or performing statistical analysis to determine quantitative changes in expression levels between a subject of unknown neuropathology and a control. In some embodiments, the method comprises depleting rRNA from the biological sample.

In some embodiments, the measurement of a circRNA DE is decreased compared to a control, then the subject is diagnosed with a neurological or neurodegenerative disease. In some embodiments, the measurement of a circRNA DE is decreased compared to a control, then the subject's disease severity has increased.

In some embodiments, the measurement of a circRNA DE is increased compared to a control, then the subject is diagnosed with a neurological or neurodegenerative disease. In some embodiments, the measurement of a circRNA DE is increased compared to a control, then the subject's disease severity has increased.

In some embodiments, the method comprises determining the severity of a neuropathological disease in a subject. In some embodiments, the subject has Alzheimer's disease (AD). In some embodiments, the biological sample is a biological fluid, blood, brain tissue, CSF, or plasma. In some embodiments, the subject does not have clinical dementia. In some embodiments, the subject is symptomatic. In some embodiments, the subject is pre-symptomatic or asymptomatic.

In some embodiments, the method comprises stratifying subjects based on disease severity. In some embodiments, the method comprises monitoring disease progression or disease severity. In some embodiments, the subject has a neurological disease or a neurodegenerative disease. In some embodiments, the neurodegenerative disease is selected from Frontotemporal dementia, Parkinson's disease dementia, and Lewy body dementia.

In some embodiments, the neurodegenerative disease is selected from AD, clinical dementia severity, or neuropathological severity. In some embodiments, primer pairs are used specifically for amplifying a backsplice junction of one or more of circHOMER1, circDOCK1, circKCNN2, circMAN2A1, circICA1, circFMN1, circRTN4, circST18, circATRNL1, and circEXOSC1.

In some embodiments, the detected circRNA is circHOMER1. In some embodiments, the primer pair is selected from the group consisting of SEQ ID NOs: 1-20. In some embodiments, the control is a biological sample from a subject without a neurodegenerative disease or neurolopathological disease.

Another aspect of the present disclosure provides for a method of identifying circRNAs associated with a neurological, neurodegenerative, or neuropathological diseases.

In some embodiments the method comprises providing or having been provided a biological samples from a subject known to have a neurological, neurodegenerative, or neuropathological disease; extract circRNA from the sample; and/or correlate circRNA to a disease state.

Another aspect of the present disclosure provides for a method of identifying circRNAs associated with a disease state comprising: generating or having been provided a discovery (disease state) dataset comprising RNA sequences of a biological sample from a subject with a disease state; generating or having been provided a replication dataset comprising RNA sequences of a biological sample from a subject; identifying disease state traits or phenotypes; processing the traits or phenotypes in the discovery dataset and/or the replication dataset; processing and alignment of the discovery dataset and/or the replication dataset; calling circRNA-defining backsplices in the discovery dataset and/or the replication dataset; filtering and collapsing annotated backsplices to identify high-confidence circRNAs in the discovery dataset and/or the replication dataset; calling linear transcripts in the discovery and/or the replication dataset; measuring transcript integrity number; and/or analyzing differential expression and correlation between the high-confidence circRNA counts and disease traits or phenotypes.

In some embodiments, the method comprises traditional linear mRNA analyses. Another aspect of the present disclosure provides for a system comprising a device capable of performing any of the preceding methods.

In an aspect, the present disclosure encompasses a method to diagnose a subject as having an increased risk for conversion to mild cognitive impairment (MCI) or dementia due to Alzheimer's disease (AD). The method comprises providing or having been provided a biological sample; extracting circRNA from the biological sample; detecting circRNA using a transcriptome-wide analysis; and/or determining the measured differential expression (DE) of the circRNA.

In another aspect, the present disclosure encompasses a method to diagnose a subject as having dementia due to Alzheimer's disease (AD). The method comprises providing or having been provided a biological sample; extracting circRNA from the biological sample; detecting circRNA using a transcriptome-wide analysis; and/or determining the measured differential expression (DE) of the circRNA.

In another aspect, the present disclosure encompasses a method to stage a subject after onset of Alzheimer's disease (AD) symptoms. The method comprises providing or having been provided a biological sample; extracting circRNA from the biological sample; detecting circRNA using a transcriptome-wide analysis; and/or determining the measured differential expression (DE) of the circRNA.

In another aspect, the present disclosure encompasses a method for treating a subject in need thereof. The method comprises providing or having been provided a biological sample; extracting circRNA from the biological sample; detecting circRNA using a transcriptome-wide analysis; and/or determining the measured differential expression (DE) of the circRNA; and (b) administering a pharmaceutical composition to the subject when the measured DE of the circRNA and/circRNA expression signature indicates pre-symptomatic or symptomatic AD relative to control reference value.

In another aspect, present disclosure encompasses a method for enrolling a subject into a clinical trial. The method comprises providing or having been provided a biological sample; extracting circRNA from the biological sample; detecting circRNA using a transcriptome-wide analysis; and/or determining the measured differential expression (DE) of the circRNA; and enrolling the subject into a clinical trial when the measured DE of the circRNA and/circRNA expression signature indicates pre-symptomatic or symptomatic AD relative to control reference value.

Other objects and features will be in part apparent and in part pointed out hereinafter.

### BRIEF DESCRIPTION OF THE FIGURES

The application file contains at least one drawing executed in color. Copies of this patent application publication with color drawing(s) will be provided by the Office upon request and payment of the necessary fee.

FIG. 1 shows cortical circRNAs are associated with AD traits. Each circular Manhattan plot presents the results from a meta-analysis of circRNA-AD association results from discovery (parietal cortex) and replication (inferior frontal gyrus (BM44)) datasets. In order from outermost to innermost circular plot, the AD traits include: CDR, Braak score and AD case-control status (AD case). The study-wide significance threshold is based on an FDR of 0.05 and depicted by the red dashed line. CircRNAs that passed this threshold are denoted with star symbols. Lines extending through all three plots identify circRNAs that are significantly associated with multiple AD traits: dotted line, two traits; solid line, three traits.

FIG. 2 shows changes in cortical circRNA expression tracks with AD clinical severity. Presented are boxplots of library-size, normalized, differential expression, covariate-adjusted counts for two AD-associated circRNAs, circHOMER1 and circCORO1C, in the Knight ADRC parietal dataset. PreSympAD (CDR≤0.5). Box plot elements: center line (median), box (first and third quartiles), whiskers (quartile±1.5×interquartile range), dots (outlier points as defined by falling outside of whiskers). AU, arbitrary units.

FIG. 3 shows AD-associated circRNAs elucidate more of the observed variation in clinical dementia rating compared with number of APOE4 alleles or the estimated proportion of neurons. Percentage of variation in CDR explained by the top 10 most meta-analysis significant CDR-associated circRNAs compared with two known contributors: number of APOE4 alleles—the most common genetic risk factor for AD—and the estimated proportion of neurons. Knight ADRC: PCtx, parietal discovery dataset (nCDR=96); MSBB BM44, inferior frontal gyrus replication dataset (nCDR=195).

FIG. 4 shows AD-associated circRNAs co-express with AD-relevant genes. Spearman's correlation-based, network co-expression module c1_46 (module association with CDR, P=1.52×10−7) in the MSBB BM44 dataset (n=195). Module association with CDR was determined from a multivariate linear regression with module eigengene and differential expression covariates. The significance of the module eigengene's association with CDR was determined using a two-tailed, Student's t-test.

FIG. 5 is a Venn diagram depicting the overlap of high-confidence circRNAs called in the different cortical RNA-seq datasets. Parietal: Knight ADRC and DIAN parietal cortex dataset (nₛₐₘₚₗₑ=170); BM10: MSBB: Brodmann Area 10 dataset (nₛₐₘₚₗₑ=265); BM22: MSBB: Brodmann Area 22 dataset (nₛₐₘₚₗₑ=264); BM36: MSBB: Brodmann Area 36 dataset (nₛₐₘₚₗₑ=267); BM44: MSBB: Brodmann Area 44 dataset (nₛₐₘₚₗₑ=230).

FIG. 6 shows Pearson's correlation plots demonstrating the imperfect correlation between a neuropathological diagnosis of definite AD (AD_case), a clinical measure of dementia—CDR—and a neuropathological measure of tau tangle pathology—Braak score (Braak). Parietal: Knight ADRC parietal cortex, discovery dataset (n_{CDR}=96, n_{Braak}=86, n_{control}=13, nAD=83); BM10: MSBB: Brodmann Area 10 replication dataset (n_{CDR}=218, n_{Braak}=209, n_{control}=46, n_{Definite} AD=108); BM22: MSBB: Brodmann Area 22 replication dataset (n_{CDR}=202, n_{Braak}=195, n_{control}=38, n_{Definite} AD=97); BM36: MSBB: Brodmann Area 36 replication dataset (n_{CDR}=187, n_{Braak}=177, n_{control}=41, n_{Definite} AD=91); BM44: MSBB: Brodmann Area 44 replication dataset (n_{CDR}=195, n_{Braak}=188, n_{control}=40, nDefinite AD=89).

FIG. 7 shows quantitative PCR validation of RNA-seq counts and direction of effect. GAPDH-normalized deltaCt values for 13 Knight ADRC discovery dataset RNA samples (n_{control}=3, n_{PreSympAD}=3, n_{AD}=7) versus RNA-seq-derived counts for the same individuals. A negative correlation is expected since circRNA transcripts with greater expression levels will reach the cycle threshold (Ct) sooner than those with lower expression and consequently have lower DeltaCt values. Shaded areas represent the 95% confidence level interval for predictions from the linear model. Corr: Pearson correlation estimates with significance determined by a two-tailed t-test. Box plot elements: center line (median), box (first and third quartiles), whiskers (quartile±1.5×interquartile range), dots (outlier points as defined by falling outside of whiskers).

FIG. 8 shows the overlap between circRNAs significantly associated with different AD traits in the Knight ADRC, parietal discovery dataset. All circRNAs were significantly associated with the AD trait at a significance less than the false discovery rate threshold of 0.05. CDR: clinical dementia rating, a clinical measure of AD severity; Braak: Braak score, a neuropathological measure of AD severity. Sample size: n_{CDR}=96, n_{Braak}=86, n_{control}=13, n_{AD}=83.

FIG. 9 shows overlap between circRNAs significantly associated with different AD traits in the meta-analysis of the Knight ADRC parietal discovery and the MSBB BM44 replication datasets. All circRNAs were significantly associated with the AD trait at a significance less than the false discovery rate threshold of 0.05. CDR: clinical dementia rating, a clinical measure of AD severity; Braak: Braak score, a neuropathological measure of AD severity measuring the number of tau tangles. Discovery, parietal sample size: n_{CDR}=96, n_{Braak}=86, n_{control}=13, n_{AD}=83; Replication, BM44 sample size: n_{CDR}=195, n_{Braak}=188, n_{control}=40, n_{Definite} AD=89.

FIG. 10 shows overlap between circRNAs significantly associated with different AD traits in the meta-analyses of the Knight ADRC parietal discovery and all four cortical regions of the MSBB replication dataset. Clinical dementia rating (CDR), Braak score, and AD case-control status. PCtx: Knight ADRC parietal cortex, discovery dataset (n_{CDR}=96, n_{Braak}=86, n_{control}=13, n_{AD}=83); BM10: MSBB: Brodmann Area 10 replication dataset (n_{CDR}=218, n_{Braak}=209, n_{control}=46, n_{Definite} AD=108); BM22: MSBB: Brodmann Area 22 replication dataset (n_{CDR}=202, n_{Braak}=195, n_{control}=38, n_{Definite} AD=97); BM36: MSBB: Brodmann Area 36 replication dataset (n_{CDR}=187, n_{Braak}=177, n_{control}=41, n_{Definite AD}=91); BM44: MSBB: Brodmann Area 44 replication dataset (n_{CDR}=195, n_{Braak}=188, n_{control}=40, n_{Definite AD}=89).

FIG. 11 shows overlap between circRNAs significantly associated with mean number of amyloid plaques, a neuropathological measure of AD severity, in the four cortical regions of the MSBB replication dataset. All circRNAs were significantly associated with mean number of plaques at the false discovery rate threshold of 0.05. BM10: MSBB Brodmann Area 10 replication dataset (n_{PlaqueMean}=218); BM22: MSBB Brodmann Area 22 replication dataset (n_{PlaqueMean}=202); BM36: MSBB Brodmann Area 36 replication dataset (n_{PlaqueMean}=187); BM44: MSBB Brodmann Area 44 replication dataset (n_{PlaqueMean}=195).

FIG. 12 shows overlap between circRNAs significantly associated with ADAD versus Braak-score-adjusted AD and versus controls (CO). ADAD versus AD* analysis was adjusted for neuropathological severity, measured by Braak score. All circRNAs were significantly associated at the false discovery rate threshold of 0.05. Sample sizes: ADADvsCO (n_{ADAD}=17, n_{control}=13); ADADvsAD* (samples with available Braak score: n_{ADAD}=17, n_{AD}=73).

FIG. 13 shows AD-associated circRNAs explain more of the observed variation in Braak score compared with number of APOE4 alleles or the estimated proportion of neurons. Percent of variation in Braak score (Braak) explained by the top 10, most meta-analysis significant Braak-associated circRNAs compared to known contributors: number of APOE4 alleles—the most common genetic risk factor for AD—and the estimated proportion of neurons. Knight ADRC: PCtx—parietal discovery dataset (n_{Braak}=86); MSBB BM44—inferior frontal gyrus replication dataset (n_{Braak}=188).

FIG. 14 shows AD-associated circRNAs improve sensitivity and specificity of logistic models predicting AD case status. The base, genetic-demographic model includes the differential expression covariates (post mortem interval, transcript integrity number, age of death, batch, sex, genetic ancestry) as well as number of APOE4 alleles. The Circ model includes normalized counts for the top 10 circRNAs most significantly associated with CDR on meta-analysis. The Circ+Base model combined the previous two models together. PCtx: Parietal discovery dataset (ncontrol=13, nAD=83); BM10: Brodmann Area 10 replication dataset (ncontrol=46, nDefinite AD=108); BM22: Brodmann Area 22 replication dataset (ncontrol=38, nDefinite AD=97); BM36: Brodmann Area 36 replication dataset (ncontrol=41, nDefinite AD=91); BM44: Brodmann Area 44 replication dataset (ncontrol=40, nDefinite AD=89).

FIG. 15 shows Backsplice junction filtering to remove artifactual junctions and generate a set of high confidence circRNA counts. Backsplice junctions detected in spiked-in, linear ERCC RNA in the Knight ADRC parietal, discovery dataset (nsamples=170) are artifactual. Two levels of filtering are depicted. The minimum number of samples filter indicates how many different samples a particular backsplice has to be observed in to be included. The minimum circular to linear (Circ: Linear) filter indicates the minimum percentage of reads classified as circular, compared to linear, a backsplice must be supported by to be included. Graph points and lines are depicted with jitter for clarity in observing overlapping points and lines.

FIG. 16 shows differential expressed circRNAs. 1A: circRNA differential expression in inferior frontal gyrus dataset between neuropathologically confirmed AD vs. neuropathologically confirmed controls. A total of 118 circRNAs are significant at a FDR of 0.05. Note x-axis depicts log2 fold change in expression. 1B: circRNA differential expression in inferior frontal gyrus dataset on the basis of clinical dementia rating (CDR). A total of 154 circRNAs are significant at an FDR of 0.05. Note x-axis depicts log2 fold change in expression per unit of CDR (range: 0-3).

FIG. 17 shows circRNA differential expression occurs early in pre-symptomatic AD and is more severe in autosomal dominant Alzheimers disease. Boxplots of circRNA normalized counts in the parietal cortex discovery dataset categorized by disease status: PreSympAD (At most, very-mild dementia), late-onset Alzheimers disease (AD), and autosomal-dominant AD (ADAD).

FIG. 18 shows 10 circRNAs provide high specificity (red line, AUC=0.88) and sensitivity for classifying brains as being from AD cases or control.

FIG. 19 shows circular RNAs can be quantified in blood and RNA and are differentially expressed.

### DETAILED DESCRIPTION

The present disclosure is based, at least in part, on the discovery that circular RNA (circRNA) can be used as a non-invasive biomarker of diseases, such as neurodegenerative disease. As outlined herein and exemplified in the Examples, the Applicants provide evidence that circRNA present in biological samples such as CSF and whole blood is differentially expressed and useful to indicate diseases in a subject. A robust and replicable differential expression (DE) of circular RNAs in brain tissues from neuropathologically-confirmed AD cases and controls was identified. circRNAs are so significantly associated with AD, that a predictive model using the counts of only 10 DE circRNAs in brain tissues provided high specificity and sensitivity (AUC: 0.88). In addition, circRNA DE was observed to be significantly associated with both clinical and pathological AD severity. Consistent with this, nominally significant circRNA DE in pre-symptomatic brains—evidence of AD pathology but no clinical dementia—compared to control brains and more severe DE in brains from individuals with ADAD was identified. Co-expression of AD-related genes with AD circRNAs through network analysis suggests a pathogenic role and therefore therapeutic compositions which directly or indirectly stabilize or reverse the DE circRNAs are useful in method of treating AD. Altogether, the present disclosure provides multiple lines of evidence linking changes in circRNA expression to AD. Thus, the present disclosure encompasses use of the methods to quantify circRNA DE to predict time to onset of mild cognitive impairment or dementia due to Alzheimer's disease, guide treatment decisions, select subjects for clinical trials, and evaluate the clinical efficacy of certain therapeutic interventions. Other aspects and iterations of the invention are described more thoroughly below.

### I. Definitions

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The term “about,” as used herein, refers to variation of in the numerical quantity that can occur, for example, through typical measuring techniques and equipment, with respect to any quantifiable variable, including, but not limited to, mass, volume, time, distance, and amount. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations, which can be up to ±5%, but can also be ±4%, 3%, 2%,1%, etc. Whether or not modified by the term “about,” the claims include equivalents to the quantities.

When introducing elements of the present disclosure or the preferred aspects(s) thereof, the articles “a,” “an,” “the,” and “said” are intended to mean that there are one or more of the elements. The terms “comprising,” “including,” and “having” are intended to be inclusive and mean that there may be additional elements other than the listed elements.

“Measuring” or “measurement,” or alternatively “detecting” or “detection,” means determining the presence, absence, quantity or amount (which can be an effective amount) of either a given substance within a clinical or subject-derived sample, including the derivation of qualitative or quantitative concentration levels of such substances, or otherwise determining the values or categorization of a subject's clinical parameters.

The terms “patient,” “subject,” “individual,” and the like are used interchangeably herein, and refer to any animal or cells thereof whether in vitro or in situ, amenable to the methods described herein. In certain non-limiting embodiments, the patient, subject or individual is a human.

“Platform” or “technology” as used herein refers to an apparatus (e.g., instrument and associated parts, computer, computer-readable media comprising one or more databases as taught herein, reagents, etc.) that may be used to measure a signature, e.g., gene expression levels, in accordance with the present disclosure. Examples of platforms include, but are not limited to, an array platform, a thermal cycler platform (e.g., multiplexed and/or real-time PCR platform), a nucleic acid sequencing platform, a hybridization and multi-signal coded (e.g., fluorescence) detector platform, etc., a nucleic acid mass spectrometry platform, a magnetic resonance platform, and combinations thereof.

In some embodiments, the platform is configured to measure gene expression levels semi-quantitatively, that is, rather than measuring in discrete or absolute expression, the expression levels are measured as an estimate and/or relative to each other or a specified marker or markers (e.g., expression of another, “standard” or “reference,” gene).

In some embodiments, semi-quantitative measuring includes “real-time PCR” by performing PCR cycles until a signal indicating the specified mRNA is detected, and using the number of PCR cycles needed until detection to provide the estimated or relative expression levels of the genes within the signature.

A real-time PCR platform includes, for example, a TaqMan® Low Density Array (TLDA), in which samples undergo multiplexed reverse transcription, followed by real-time PCR on an array card with a collection of wells in which real-time PCR is performed. A real-time PCR platform also includes, for example, a Biocartis Idylla™ sample-to-result technology, in which cells are lysed, DNA/RNA extracted and real-time PCR is performed and results detected. A real-time PCR platform also includes, for example, CyTOF analysis: CyTOF (Fludigm) is a recently introduced mass-cytometer capable of detecting up to 40 markers conjugated to heavy metals simultaneously on single cells.

A magnetic resonance platform includes, for example, T2 Biosystems® T2 Magnetic Resonance (T2MR®) technology, in which molecular targets may be identified in biological samples without the need for purification.

The terms “array,” “microarray” and “micro array” are interchangeable and refer to an arrangement of a collection of nucleotide sequences presented on a substrate. Any type of array can be utilized in the methods provided herein. For example, arrays can be on a solid substrate (a solid phase array), such as a glass slide, or on a semi-solid substrate, such as nitrocellulose membrane. Arrays can also be presented on beads, i.e., a bead array. These beads are typically microscopic and may be made of, e.g., polystyrene. The array can also be presented on nanoparticles, which may be made of, e.g., particularly gold, but also silver, palladium, or platinum. See, e.g., Nanosphere Verigene® System, which uses gold nanoparticle probe technology. Magnetic nanoparticles may also be used. Other examples include nuclear magnetic resonance microcoils. The nucleotide sequences can be DNA, RNA, or any permutations thereof (e.g., nucleotide analogues, such as locked nucleic acids (LNAs), and the like). In some embodiments, the nucleotide sequences span exon/intron boundaries to detect gene expression of spliced or mature RNA species rather than genomic DNA. The nucleotide sequences can also be partial sequences from a gene, primers, whole gene sequences, non-coding sequences, coding sequences, published sequences, known sequences, or novel sequences. The arrays may additionally comprise other compounds, such as antibodies, peptides, proteins, tissues, cells, chemicals, carbohydrates, and the like that specifically bind proteins or metabolites.

An array platform includes, for example, the TaqMan® Low Density Array (TLDA) mentioned above, and an Affymetrix® microarray platform.

A hybridization and multi-signal coded detector platform includes, for example, NanoString nCounter® technology, in which hybridization of a color-coded barcode attached to a target-specific probe (e.g., corresponding to a gene expression transcript of interest) is detected; and Luminex® xMAP® technology, in which microsphere beads are color coded and coated with a target-specific (e.g., gene expression transcript) probe for detection; and IIlumina® BeadArray, in which microbeads are assembled onto fiber optic bundles or planar silica slides and coated with a target-specific (e.g., gene expression transcript) probe for detection.

A nucleic acid mass spectrometry platform includes, for example, the Ibis Biosciences Plex-ID® Detector, in which DNA mass spectrometry is used to detect amplified DNA using mass profiles.

A thermal cycler platform includes, for example, the FilmArray® multiplex PCR system, which extract and purifies nucleic acids from an unprocessed sample and performs nested multiplex PCR; the RainDrop Digital PCR System, which is a droplet-based PCR platform using microfluidic chips; and the GenMark eSensor or ePlex systems.

The term “genetic material” refers to a material used to store genetic information in the nuclei or mitochondria of an organism's cells. Examples of genetic material include, but are not limited to, double-stranded and single-stranded DNA, cDNA, RNA, and mRNA.

The term “plurality of nucleic acid oligomers” refers to two or more nucleic acid oligomers, which can be DNA or RNA.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

“Aβ amyloidosis” is clinically defined as evidence of Aβ deposition in the brain. A subject that is clinically determined to have Aβ amyloidosis is referred to herein as “amyloid positive,” while a subject that is clinically determined to not have Aβ amyloidosis is referred to herein as “amyloid negative.” Aβ□amyloidosis likely exists before it is detectable by current techniques. Nonetheless, there are accepted indicators of Aβ amyloidosis in the art. At the time of this disclosure, Aβ amyloidosis is typically identified by amyloid imaging (e.g., PiB PET, fluorbetapir, or other imaging methods known in the art) or by decreased cerebrospinal fluid (CSF) Aβ42 or a decreased CSF Aβ42/40 ratio. [11C]PIB-PET imaging with mean cortical binding potential (MCBP) score>0.18 is an indicator of Aβ amyloidosis, as is cerebral spinal fluid (CSF) Aβ42 concentration of about 1 ng/ml by immunoprecipitation and mass spectrometry (IP/MS)). Values such as these, or others known in the art, may be used alone or in combination to clinically confirm Aβ□amyloidosis. See, for example, Klunk W E et al. Ann Neurol 55(3) 2004, Fagan A M et al. Ann Neurol, 2006, 59(3), Patterson et. al, Annals of Neurology, 2015, 78(3): 439-453, or Johnson et al., J. Nuc. Med., 2013, 54(7): 1011-1013, each hereby incorporated by reference in its entirety. Subjects with Aβ amyloidosis may or may not be symptomatic, and symptomatic subjects may or may not satisfy the clinical criteria for a disease associated with Aβ amyloidosis. Non-limiting examples of symptoms associated with Aβ amyloidosis may include impaired cognitive function, altered behavior, abnormal language function, emotional dysregulation, seizures, dementia, and impaired nervous system structure or function. Diseases associated with Aβ amyloidosis include, but are not limited to, Alzheimer's Disease (AD), cerebral amyloid angiopathy, Lewy body dementia, and inclusion body myositis. Subjects with Aβ amyloidosis are at an increased risk of developing a disease associated with Aβ amyloidosis.

A “clinical sign of Aβ amyloidosis” refers to a measure of Aβ deposition known in the art. Clinical signs of Aβ amyloidosis may include, but are not limited to, Aβ deposition identified by amyloid imaging (e.g. PiB PET, fluorbetapir, or other imaging methods known in the art) or by decreased cerebrospinal fluid (CSF) Aβ42 or Aβ42/40 ratio. See, for example, Klunk W E et al. Ann Neurol 55(3) 2004, and Fagan A M et al. Ann Neurol 59(3) 2006, each hereby incorporated by reference in its entirety. Clinical signs of Aβ amyloidosis may also include measurements of the metabolism of Aβ, in particular measurements of Aβ42 metabolism alone or in comparison to measurements of the metabolism of other Aβ variants (e.g. Aβ37, Aβ38, Aβ39, Aβ40, and/or total Aβ), as described in U.S. patent Ser. Nos. 14/366,831, 14/523,148 and 14/747,453, each hereby incorporated by reference in its entirety. Additional methods are described in Albert et al. Alzheimer's & Dementia 2007 Vol. 7, pp. 170-179; McKhann et al., Alzheimer's & Dementia 2007 Vol. 7, pp. 263-269; and Sperling et al. Alzheimer's & Dementia 2007 Vol. 7, pp. 280-292, each hereby incorporated by reference in its entirety. Importantly, a subject with clinical signs of Aβ amyloidosis may or may not have symptoms associated with Aβ deposition. Yet subjects with clinical signs of Aβ amyloidosis are at an increased risk of developing a disease associated with Aβ amyloidosis.

A “candidate for amyloid imaging” refers to a subject that has been identified by a clinician as an individual for whom amyloid imaging may be clinically warranted. As a non-limiting example, a candidate for amyloid imaging may be a subject with one or more clinical signs of Aβ amyloidosis, one or more Aβ plaque associated symptom, one or more CAA associated symptom, or combinations thereof. As a non-limiting example, a candidate for amyloid imaging may be a subject with genetic predisposition for Aβ amyloidosis. A clinician may recommend amyloid imaging for such a subject to direct his or her clinical care. As another non-limiting example, a candidate for amyloid imaging may be a potential participant in a clinical trial for a disease associated with Aβ amyloidosis (either a control subject or a test subject).

An “Aβ plaque associated symptom” or a “CAA associated symptom” refers to any symptom caused by or associated with the formation of amyloid plaques or CAA, respectively, being composed of regularly ordered fibrillar aggregates called amyloid fibrils. Exemplary Aβ plaque associated symptoms may include, but are not limited to, neuronal degeneration, impaired cognitive function, impaired memory, altered behavior, emotional dysregulation, seizures, impaired nervous system structure or function, and an increased risk of development or worsening of Alzheimer's disease or CAA. Neuronal degeneration may include a change in structure of a neuron (including molecular changes such as intracellular accumulation of toxic proteins, protein aggregates, etc. and macro level changes such as change in shape or length of axons or dendrites, change in myelin sheath composition, loss of myelin sheath, etc.), a change in function of a neuron, a loss of function of a neuron, death of a neuron, or any combination thereof. Impaired cognitive function may include but is not limited to difficulties with memory, attention, concentration, language, abstract thought, creativity, executive function, planning, and organization. Altered behavior may include, but is not limited to, physical or verbal aggression, impulsivity, decreased inhibition, apathy, decreased initiation, changes in personality, abuse of alcohol, tobacco or drugs, and other addiction-related behaviors. Emotional dysregulation may include, but is not limited to, depression, anxiety, mania, irritability, and emotional incontinence. Seizures may include but are not limited to generalized tonic-clonic seizures, complex partial seizures, and non-epileptic, psychogenic seizures. Impaired nervous system structure or function may include, but is not limited to, hydrocephalus, Parkinsonism, sleep disorders, psychosis, impairment of balance and coordination. This may include motor impairments such as monoparesis, hemsiparesis, tetraparesis, ataxia, ballismus and tremor. This also may include sensory loss or dysfunction including olfactory, tactile, gustatory, visual and auditory sensation. Furthermore, this may include autonomic nervous system impairments such as bowel and bladder dysfunction, sexual dysfunction, blood pressure and temperature dysregulation. Finally, this may include hormonal impairments attributable to dysfunction of the hypothalamus and pituitary gland such as deficiencies and dysregulation of growth hormone, thyroid stimulating hormone, lutenizing hormone, follicle stimulating hormone, gonadotropin releasing hormone, prolactin, and numerous other hormones and modulators.

As used herein, the term “subject” refers to a mammal, preferably a human. The mammals include, but are not limited to, humans, primates, livestock, rodents, and pets. A subject may be waiting for medical care or treatment, may be under medical care or treatment, or may have received medical care or treatment.

As used herein, the term “healthy control group,” “normal group” or a sample from a “healthy” subject means a subject, or group subjects, who is/are diagnosed by a physician as not suffering from Aβ amyloidosis, or a clinical disease associated with Aβ amyloidosis (including but not limited to Alzheimer's disease) based on qualitative or quantitative test results. A “normal” subject is usually about the same age as the individual to be evaluated, including, but not limited, subjects of the same age and subjects within a range of 5 to 10 years.

As used herein, the term “blood sample” refers to a biological sample derived from blood, preferably peripheral (or circulating) blood. The blood sample can be whole blood, plasma or serum, although plasma is typically preferred.

“Significantly deviate from the mean” refers to values that are at least 1 standard deviation, preferably at least 1.3 standard deviations, more preferably at least 1.5 standard deviations or even more preferably at least 2 standard deviations, above or below the mean.

### II. Methods

One aspect of the present disclosure encompasses a method for diagnosing a disease of a subject, comprising the step of: determining the presence or absence of one or more circular RNA (circRNA) in a biological sample (e.g. a bodily fluid) of said subject; wherein the presence or absence of said one or more circRNA is indicative for the disease. Preferably, said disease is not a disease of a bodily fluid. Certain circRNAs may be present in samples of a diseased subject at differing levels as compared to samples from healthy subjects or at differing levels based on disease severity of the subject. Therefore, the present disclosure encompasses determining the “presence” or “absence” of a circRNA when compared to a reference level and/or determining the level of said one or more circRNA; comparing the determined level to a reference level of said one or more circRNA. Thus, the present disclosure provides the steps of determining the level of said one or more circRNA; comparing the determined level to a reference level of said one or more circRNA; wherein differing levels between the determined and the reference level are indicative for the disease. Hence, the invention also relates to a method for diagnosing a disease of a subject or the disease severity of a subject, comprising the step of: determining the level of said one or more circRNA; comparing the determined level to a reference level of said one or more circRNA; wherein differing levels between the determined and the reference level are indicative for the disease or disease severity.

In an embodiment of the present invention, the method is a method for diagnosing a neurodegenerative disease such as Alzheimer's disease, Frontotemporal dementia, Parkinsons disease dementia, and Lewy body dementia in a subject. In some embodiments the subject has a diagnosis of MCI or dementia. In some embodiments the subject has no clinical signs of a neurodegenerative disease.

The methods as disclosed herein generally comprise providing or having been provided a biological sample. As used herein, the term “biological sample” means a biological material isolated from a subject. Any biological sample containing any biological material suitable for detecting the desired circRNAs, and may comprise cellular and/or non-cellular material obtained from the subject is suitable. Non-limiting examples include blood, plasma, serum, urine, and tissue. Frequently the sample will be a “clinical sample” which is a sample derived from a patient. Typical clinical samples include, but are not limited to, bodily fluid samples such as synovial fluid, sputum, blood, urine, blood plasma, blood serum, sweat, mucous, saliva, lymph, bronchial aspirates, peritoneal fluid, cerebrospinal fluid, and pleural fluid, and tissues samples such as blood-cells (e.g., white cells), tissue or fine needle biopsy samples and abscesses or cells therefrom. Biological samples may also include sections of tissues, such as frozen sections or formalin fixed sections taken for histological purposes. In some embodiments, the biological sample is selected from CSF, blood, or brain tissues.

A “sample” may also be a sample originating from a biochemical or chemical reaction such as the product of an amplification reaction. Liquid samples may be subjected to one or more pre-treatments prior to use in the present disclosure. Such pre-treatments include, but are not limited to dilution, filtration, centrifugation, concentration, sedimentation, precipitation or dialysis. Pre-treatments may also include the addition of chemical or biochemical substances to the solution, e.g. in order to stabilize the sample and the contained nucleic acids, in particular the circRNAs. Such addition of chemical or biochemical substances include acids, bases, buffers, salts, solvents, reactive dyes, detergents, emulsifiers, or chelators, like EDTA. The sample may for instance be taken and directly mixed with such substances. In one embodiment the sample is a whole blood sample. The whole blood sample is preferably not pre-treated by means of dilution, filtration, centrifugation, concentration, sedimentation, precipitation or dialysis. It is, however, preferred that substances are added to the sample in order to stabilize the sample until onset of analysis. “Stabilizing” in this context means prevention of degradation of the circRNAs to be determined. Preferred stabilizers in this context are EDTA, e.g. K2EDTA, RNase inhibitors, alcohols e.g. ethanol and isopropanol, agents used to salt out proteins (such as RNAlater). In one aspect, the sample is treated to remove rRNA (ribosomal RNA). Techniques to deplete rRNA from samples are known in the art, for example RiboMinus technology.

As will be appreciated by a skilled artisan, the method of collecting a biological sample can and will vary depending upon the nature of the biological sample and the type of analysis to be performed. Any of a variety of methods generally known in the art may be utilized to collect a biological sample. Generally speaking, the method preferably maintains the integrity of the sample such that the circRNA can be accurately detected and the amount measured according to the disclosure.

In some embodiments, a single sample is obtained from a subject to detect one or more circRNAs in the sample. Alternatively, one or more circRNAs may be detected in samples obtained over time from a subject. As such, more than one sample may be collected from a subject over time. For instance, 2, 3, 4, 5, 6, 7, 8, 9, 10, 11, 12, 13, 14, 15, 16 or more samples may be collected from a subject over time. In some embodiments, 2, 3, 4, 5, or 6 samples are collected from a subject over time. In other embodiments, 6, 7, 8, 9, or 10 samples are collected from a subject over time. In yet other embodiments, 10, 11, 12, 13, or 14 samples are collected from a subject over time. In other embodiments, 14, 15, 16 or more samples are collected from a subject over time.

When more than one sample is collected from a subject over time, samples may be collected every 0.5, 1, 2, 3, 4, 5, 6, 7, 8, 9, 10, 11, 12 or more hours. In some embodiments, samples are collected every 0.5, 1, 2, 3, or 4 hours. In other embodiments, samples are collected every 4, 5, 6, or 7 hours. In yet other embodiments, samples are collected every 7, 8, 9, or 10 hours. In other embodiments, samples are collected every 10, 11, 12 or more hours. Additionally, samples may be collected every 1, 2, 3, 4, 5, 6, 7, 8, 9, 10, 11, 12 or more days. In some embodiments, a sample is collected about every 6 days. In some embodiments, samples are collected every 1, 2, 3, 4, or 5 days. In other embodiments, samples are collected every 5, 6, 7, 8, or 9 days. In yet other embodiments, samples are collected every 9, 10, 11, 12 or more days.

In some embodiments the sample comprises a nucleic acid or nucleic acids. The term “nucleic acid” is here used in its broadest sense and comprises ribonucleic acids (RNA) and deoxyribonucleic acids (DNA) from all possible sources, in all lengths and configurations, such as double stranded, single stranded, circular, linear or branched. All sub-units and subtypes are also comprised, such as monomeric nucleotides, oligomers, plasmids, viral and bacterial nucleic acids, as well as genomic and non-genomic DNA and RNA from the subject, circular RNA (circRNA), messenger RNA (mRNA) in processed and unprocessed form, transfer RNA (tRNA), heterogeneous nuclear RNA (hn-RNA), ribosomal RNA (rRNA), complementary DNA (cDNA) as well as all other conceivable nucleic acids. However, in the most preferred embodiment the sample comprises circRNAs. “Presence” or “absence” of a circRNA in connection with the present disclosure means that the circRNA is present at levels above a certain threshold or below a certain threshold, respectively. In case the threshold is “0” this would mean that “presence” is the actual presence of circRNA in the sample and “absence” is the actual absence. However, “presence” in context with the present disclosure may also mean that the respective circRNA is present at a level above a threshold, e.g. the levels determined in a control, “absence” in this context then means that the level of the circRNA is at or below the certain threshold. Hence, it is preferred that the method of the present disclosure comprises determining of the level of one or more circRNA and comparing it to a reference level of said one or more circRNA. In one embodiment the determination step comprises: (i) determining the level of said one or more circRNA; and (ii) comparing the determined level to a reference level of said one or more circRNA; wherein differing levels between the determined and the reference level are indicative for the disease. In other words, the disclosure relates to a method for diagnosing a disease of a subject, comprising the step of (i) determining the level of said one or more circRNA; and (ii) comparing the determined level to a reference level of said one or more circRNA; wherein differing levels between the determined and the reference level are indicative for the disease.

The term “reference level” relates to a level to which the determined level is compared in order to allow the distinction between “presence” or “absence” of the circRNA. The reference level includes the level which is determinant for the deductive step of making the actual diagnose. The reference level in a preferred embodiment relates to the level of the respective circRNA in a healthy subject or a population of healthy subjects, i.e. a subject not having the disease to be diagnosed, e.g. not having a neurodegenerative disease, such as Alzheimer's disease. The skilled person with the disclosure of the present application is in the position to determine suited control levels using common statistical methods.

A “reference level” of a circRNA may also mean a level of the circRNA that is indicative of the absence of a particular disease state or disease severity. In some embodiments, when the level of a circRNA in a subject is above the reference level of the circRNA it is indicative of the presence of a disease state or increased disease severity. In some embodiments, when the level of a circRNA in a subject is above the reference level of the circRNA it is indicative of the lack of a particular disease state or lack of severity of the disease. In some embodiments, when the level of a circRNA in a subject is below the reference level of the circRNA it is indicative of the presence of a particular disease state or increased severity of the disease. In some embodiments, when the level of a circRNA in a subject is below the reference level of the circRNA it is indicative of the lack of a particular disease state or lack of severity of the disease. In some embodiments, when the level of a circRNA in a subject is within the reference level of the circRNA it is indicatives a particular disease state or severity.

As used herein, the term “indicative” when used with circRNA expression levels, means that the circRNA expression levels are up-regulated or down-regulated, altered, or changed compared to the expression levels in alternative biological states (e.g., asymptomatic, pre-symptomatic, MCI, dementia) or control.

In certain embodiments, to classify the amount of one or more circRNAs as increased in a biological sample, the amount of the one or more circRNAs in the biological sample compared to the reference value is increased at least 1-fold. For example, the amount of the one or more circRNAs in the sample compared to the reference value is increased at least 1-fold, at least 1.25-fold, at least 1.5-fold, at least 1.75-fold, at least 2-fold, at least 5-fold, at least 10-fold, at least 15-fold, at least 20-fold, at least 25-fold, at least 30-fold, at least 35-fold, at least 40-fold, at least 45-fold, at least 50-fold, at least 100-fold, at least 200-fold, at least 300-fold, at least 400-fold, at least 500-fold, at least 1000-fold, at least 5000-fold, or at least 10000-fold.

In certain embodiments, to classify the amount of one or more circRNAs as decreased in a biological sample, the amount of the one or more circRNAs in the biological sample compared to the reference value is decreased at least 1-fold. For example, the amount of the one or more circRNAs in the sample compared to the reference value is decreased at least 1-fold, at least 1.25-fold, at least 1.5-fold, at least 1.75-fold, at least 2-fold, at least 5-fold, at least 10-fold, at least 15-fold, at least 20-fold, at least 25-fold, at least 30-fold, at least 35-fold, at least 40-fold, at least 45-fold, at least 50-fold, at least 100-fold, at least 200-fold, at least 300-fold, at least 400-fold, at least 500-fold, at least 1000-fold, at least 5000-fold, or at least 10000-fold.

In another embodiment, the increase or decrease in the amount of a host analyte is measured using p-value. For instance, when using p-value, a circRNA is identified as being differentially expressed between a biological sample and a reference value when the p-value is less than 0.1, preferably less than 0.05, more preferably less than 0.01, even more preferably less than 0.005, the most preferably less than 0.001.

The levels of the one or more circRNA may be analyzed in a number of fashions well known to a person skilled in the art. For example, each assay result obtained may be compared to a “normal” or “control” value, or a value indicating a particular disease or outcome. A particular diagnosis/prognosis may depend upon the comparison of each assay result to such a value, which may be referred to as a diagnostic or prognostic “threshold”. In certain embodiments, assays for one or more diagnostic or prognostic indicators are correlated to a condition or disease by merely the presence or absence of the circRNAs in the assay. For example, an assay can be designed so that a positive signal only occurs above a particular threshold level of interest, and below which level the assay provides no signal above background.

The sensitivity and specificity of a diagnostic and/or prognostic test depends on more than just the analytical “quality” of the test, they also depend on the definition of what constitutes an abnormal result, i.e. when a level may be regarded as differing from a control level. In practice, Receiver Operating Characteristic curves (ROC curves), are typically calculated by plotting the value of a variable versus its relative frequency in “normal” (i.e. apparently healthy individuals not having ovarian cancer) and “disease” populations. For any particular marker, a distribution of marker levels for subjects with and without a disease will likely overlap. Under such conditions, a test does not absolutely distinguish normal from disease with 100% accuracy, and the area of overlap indicates where the test cannot distinguish normal from disease. A threshold is selected, below which the test is considered to be abnormal and above which the test is considered to be normal. The area under the ROC curve is a measure of the probability that the perceived measurement will allow correct identification of a condition. ROC curves can be used even when test results don't necessarily give an accurate number. As long as one can rank results, one can create a ROC curve. For example, results of a test on “disease” samples might be ranked according to degree (e.g. 1=low, 2=normal, and 3=high). This ranking can be correlated to results in the “normal” or “control” population, and a ROC curve created. These methods are well known in the art. See, e.g., Hartley et al. 1982. Radiology 143: 29-36. Preferably, a threshold is selected to provide a ROC curve area of greater than about 0.5, more preferably greater than about 0.7, still more preferably greater than about 0.8, even more preferably greater than about 0.85, and most preferably greater than about 0.9. The term “about” in this context refers to +/−5% of a given measurement.

The horizontal axis of the ROC curve represents (1-specificity), which increases with the rate of false positives. The vertical axis of the curve represents sensitivity, which increases with the rate of true positives. Thus, for a particular cut-off selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test. In other embodiments, a positive likelihood ratio, negative likelihood ratio, odds ratio, or hazard ratio is used as a measure of a test's ability to predict risk or diagnose a disease. In the case of a positive likelihood ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In the case of a negative likelihood ratio, a value of 1 indicates that a negative result is equally likely among subjects in both the “diseased” and “control” groups; a value greater than 1 indicates that a negative result is more likely in the test group; and a value less than 1 indicates that a negative result is more likely in the control group.

In the case of an odds ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group.

In the case of a hazard ratio, a value of 1 indicates that the relative risk of an endpoint (e.g., death) is equal in both the “diseased” and “control” groups; a value greater than 1 indicates that the risk is greater in the diseased group; and a value less than 1 indicates that the risk is greater in the control group.

The skilled artisan will understand that associating a diagnostic or prognostic indicator, with a diagnosis or with a prognostic risk of a future clinical outcome is a statistical analysis. For example, a marker level of lower than X may signal that a patient is more likely to suffer from an adverse outcome than patients with a level more than or equal to X, as determined by a level of statistical significance. For another marker, a marker level of higher than X may signal that a patient is more likely to suffer from an adverse outcome than patients with a level less than or equal to X, as determined by a level of statistical significance. Additionally, a change in marker concentration from baseline levels may be reflective of patient prognosis, and the degree of change in marker level may be related to the severity of adverse events. Statistical significance is often determined by comparing two or more populations, and determining a confidence interval and/or a p value. See, e.g., Dowdy and Wearden, Statistics for Research, John Wiley & Sons, New York, 1983. Preferred confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001. Suitable threshold levels for the diagnosis of the disease can be determined for certain combinations of circRNAs. This can e.g. be done by grouping a reference population of patients according to their level of circRNAs into certain quantiles, e.g. quartiles, quintiles or even according to suitable percentiles. For each of the quantiles or groups above and below certain percentiles, hazard ratios can be calculated comparing the risk for an adverse outcome, i.e. a “disease” or “Alzheimer's disease”, between those patients who have a certain disease and those who have not. In such a scenario, a hazard ratio (HR) above 1 indicates a higher risk for an adverse outcome for the patients. A HR below 1 indicates beneficial effects of a certain treatment in the group of patients. A HR around 1 (e.g. +/−0.1) indicates no elevated risk for the particular group of patients. By comparison of the HR between certain quantiles of patients with each other and with the HR of the overall population of patients, it is possible to identify those quantiles of patients who have an elevated risk and those who benefit from medication and thereby stratify subjects according to the present invention. In some cases presence of the disease will not affect patients with levels (e.g. in the fifth quintile) of a circRNA different from the “reference level”, while in other cases patients with levels similar to the control level will be affected (e.g. in the first quintile). However, with the above explanations and his common knowledge, a skilled person is able to identify those groups of patients having a disease, e.g. a neurodegenerative disease as Alzheimer's disease. Exemplarily, some combinations of levels of circRNAs are listed for Alzheimer's disease in the appended examples. In another embodiment of the invention, the diagnosis is determined by relating the patient's individual level of marker peptide to certain percentiles (e.g. 97.5th percentile (in case increased levels being indicative for a disease) or the 2.5th percentile (in case decreased levels being indicative for a disease)) of a healthy population.

Kaplan-Meier estimators may be used for the assessment or prediction of the outcome or risk (e.g. diagnosis, relapse, progression or morbidity) of a patient.

“Equal” level in context with the present invention means that the levels differ by not more than ±10%, preferably by not more than ±5%, more preferably by not more than ±2%.“Decreased” or “increased” level in the context of the present invention mean that the levels differ by more than 10%, preferably by more than 15%, preferably more than 20%.

“Circular RNA” (circRNA) has been previously described. The skilled person is able to determine whether a detected RNA is a circular RNA. In particular, a circRNA does not contain a free 3 ‘-end or a free 5’ end, i.e. the entire nucleic acid is circularized. The circRNA is preferably a circularized, single stranded RNA molecule. Furthermore, the circRNA is a result of a 5′ to 3′ splicing event that results in a discontinuous sequence with respect to the genomic sequence encoding the RNA. This means that a first sequence being present 5′-upstream of a second sequence in the genomic context, on the circRNA said first sequence at its 5′-end is linked to the 3′ end of said second sequence and thereby closing the circle. The consequence of this arrangement is that at the junction where the 5′-end of said first sequence is linked to the 3′-end of said second sequence a unique sequence is build that is neither present in the genomic context nor in the normally transcribed RNA, e.g. mRNA. Thus, the circRNAs according to the disclosure preferably contain a backsplice junction in a head-to-tail arrangement. The skilled person will recognize that a usual mRNA transcript contains exon-exon junctions in a tail-to-head arrangement, i.e. the 3′end (tail) of exon being upstream in the genomic context is linked to the 5′end (head) of the exon being downstream in the genomic context. The actual junction, i.e. the point at which the one exon is linked to the other is also referred to herein as “breakpoint”. In a preferred embodiment the presence or absence of a circRNA or the level of a circRNA is determined by detection of a backsplice junction. In some embodiments, the methods disclosed herein comprise detecting one or more circRNAs by contacting the sample with one or more of the following primer pairs designed to the backsplice junctions of circHOMER1 (forward 5′-TTTGGAAGACATGAGCTCGA-3′(SEQ ID NO:1); reverse 5′-AAGGGCTGAACCAACTCAGA-3′(SEQ ID NO:2)), circKCNN2 (forward 5′-GACTGTCCGAGCTTGTGAAA-3′(S EQ ID N0:3); reverse 5′-GGCCGTCCATGTGAATGTAT-3′(SEQ ID NO:4)), circMAN2A1 (forward 5′-TGAAAGAAGACTCACGGAGGA-3′(SEQ ID NO:5); reverse 5′-TAGCAAACGCTCCAAATGGT-3′(SEQ ID NO:6)), circICA1 (forward 5′-TTGATGATTTGGGGAGAAGG-3′(SEQ ID NO:7); reverse 5′-TGGATGAAGGACGTGTCTCA-3′(SEQ ID NO:8)), circFMN1 (forward 5′-GGTGGCTATGCAGAGAAAGC-3′(SEQ ID NO:9); reverse 5′-CAGGGAAGACCACAGCTGAG-3′(SEQ ID NO:10)); circDOCK1 (forward 5′-AGCTGAGGGACAACAACACC-3′(SEQ ID NO:11); reverse 5′-GGCCGTCCATGTGAATGTAT-3′(SEQ ID NO:12)), circRTN4 (forward 5′-TGAAAGCAGCAGGAATAGGC-3′(SEQ ID NO:13); reverse 5′-CAGGCGCCTCTTCTTAGTTG-3′(SEQ ID NO:14)), circST18 (forward 5′-CTGGAGTTTTCTTGTGCAGTTG-3′(SEQ ID NO:15); reverse 5′-AAACCCAAGCTTCATGCAAG-3′(SEQ ID NO:16)), circIATRNL1 (forward 5′-GGGTATAAAGCATTGCCAGG-3′(SEQ ID NO:17); reverse 5′-GCCTTCAATGAGCCAAGTACA-3′(SEQ ID NO:18)), circEXOSC1 (forward 5′-CTTTGGCCAAGACAATGTCA-3′(SEQ ID NO:19); reverse 5′-GTGTGAGATGCAGTGCCCTA-3′(SEQ ID NO:20)). In some embodiments, the methods include the use of primers which comprise, consist essentially of, or consist of the nucleotide sequence of SEQ ID NOs: 1-20.

The detection of circular RNA has also been previously described in Memczak S, Jens M, Elefsinioti A, et al. Circular RNAs are a large class of animal RNAs with regulatory potency. Nature. 2013; 495(7441):333-338, which is incorporated herein by reference in particular as relates to the detection and annotation of circRNAs. The biogenesis of many mammalian circRNAs depends on complementary sequences within flanking introns {see Ashwal-Fluss R, Meyer M, Pamudurti N R, et al. circRNA Biogenesis Competes with Pre-mRNA Splicing. MOLCEL. 2014; 1-12; Rybak-Wolf A, Stottmeister C, Glazar P, et al. Circular RNAs in the Mammalian Brain Are Highly Abundant, Conserved, and Dynamically Expressed. MOLCEL. 2015;1-17; Zhang X-O, Wang H-B, Zhang Y, et al. Complementary Sequence-Mediated Exon Circularization. Cell. 2014; Liang D, Wilusz J E. Short intronic repeat sequences facilitate circular RNA production. Genes and Development. 2014; Conn S J, Pillman K A, Toubia J, et al. The RNA Binding Protein Quaking Regulates Formation of circRNAs. Cell. 2015; 160(6): 1 125-1 134; and Ivanov A, Memczak S, Wyler E, et al. Analysis of Intron Sequences Reveals Hallmarks of Circular RNA Biogenesis in Animals. CellReports. 2015; 10(2): 170-177). Hence, in one embodiment the two introns upstream and downstream of and direct adjacent in the genomic context to the exons of the exon-exon junction (i.e. forming the exon-exon junction) in a head to tail arrangement often contain complementary sequences, e.g. a complementary sequence stretch of at least 15 nucleotides, preferably 500 nucleotides, more preferably 1000 nucleotides. For detection of circRNA in principle, the RNA of a sample is sequenced after reverse transcription and library preparation. Afterwards, the sequences are analyzed for the presence of exon-exon junctions in a head-to-tail arrangement i.e. backsplice junctions. For instance RNA sequenced can be mapped to a reference genome using common mapping programs and software, e.g. bowtie2 (version 2.1.0; see Langmead B, Salzberg S L. Fast gapped-read alignment with Bowtie 2. Nature Methods. 2012; 9(4):357-359). Human reference genomes are known to the skilled person and include the human reference genome hg19 (February 2009, GRCh37; downloadable from the UCSC genome browser; see Kent W J, Sugnet C W, Furey T S, et al. The human genome browser at UCSC. Genome Research. 2002; 12(6):996-1006). Although circRNA detection in blood is possible without any preprocessing of the total RNA sample, it is preferred to deplete ribosomal RNAs (rRNA), preferably the majority of rRNA, to increase the sensitivity of circRNA detection, in particular when using RNA Sequencing approaches. To this end, the content of rRNAin the sample should be depleted to less than 20%, preferably less than 10%, more preferably less than 2% with respect to the total RNA content. The rRNA depletion may performed as known in the art, e.g. it may be facilitated by commercially available kits (e.g. Ribominus, Themo Scientific) or enzymatic methods (Xian Adiconis et al. Comprehensive comparative analysis of RNA sequencing methods for degraded or low input samples Nat Methods. 2013 July; 10(7): 10.1038/nmeth.2483.). Further, in a preferred embodiment RNA sequences which map continuously to the genome by aligning without any trimming (end-to-end mode) are neglected. Reads not mapping continuously to the genome are preferably used for circRNA candidate detection. The terminal sequences (anchors) from the sequences, e.g. 20 nt or more, may be extracted and re-aligned independently to the genome. From this alignment the sequences may be extended until the full circRNA sequence is covered, i.e. aligned. Consecutively aligning anchors indicate linear splicing events whereas alignment in reverse orientation indicates head-to-tail splicing as observed in circRNAs.

The circRNAs according to the present invention may be detected using different techniques. As outlined herein, the backsplice arrangement is unique to the circRNAs. Hence, the detection of these is preferred. Nucleic acid detection methods are commonly known to the skilled person and include probe hybridization based methods, nucleic acid amplification based methods, and nucleic acid sequencing, or combinations thereof. Hence, in a preferred embodiment of the present invention circRNA is detected using a method selected from the group consisting of probe hybridization based methods, nucleic acid amplification based methods, and nucleic acid sequencing.

Probe hybridization based method employ the feature of nucleic acids to specifically hybridize to a complementary strand. To this end nucleic acid probes may be employed that specifically hybridize to the exon-exon junction in a head-to-tail arrangement of the circRNA, i.e. to a sequence spanning the exon-exon junction, preferably to the region extending from 10 nt upstream to 10 nt downstream of the exon-exon junction, preferably to the region from 20 nt upstream to 20 nt downstream of the exon-exon junction, or even a greater region spanning the exon-exon junction. The skilled person will recognize that hybridization probes specifically hybridizing to the respective sequence of the circRNA may be used, as well as hybridization probes specifically hybridizing to the reverse complement sequence thereof, e.g. in case the circRNA is previously reverse transcribed to cDNA and/or amplified.

Hybridization can also be used as a measure of homology between two nucleic acid sequences. A nucleic acid sequence hybridizing specifically to an exon-exon junction in a head-to-tail arrangement according to the present invention may be used as a hybridization probe according to standard hybridization techniques. The hybridization of the probe to DNA or RNA from a test source (e.g., the bodily fluid, like whole blood, or amplified nucleic acids from the sample of the bodily fluid) is an indication of the presence of the relevant circRNA in the test source.

Hybridization conditions are known to those skilled in the art and can be found in Current Protocols in Molecular Biology, John Wiley & Sons, N.Y., 6.3.1-6.3.6, 1991. Preferably, specific hybridization refers to hybridization under stringent conditions. “Stringent conditions” are defined as equivalent to hybridization in 6×sodium chloride/sodium citrate (SSC) at 45° C., followed by a wash in 0.2×SSC, 0.1% SDS at 65° C.; or as equivalent to hybridization in commercially available hybridization buffers (e.g. ULTRAHyb, ThermoScientific) for blotting techniques and 5×SSC 0.5% SDS (750 niM NaCl, 75 mM sodium citrate, 0.5% sodiumdodecylsulfate, pH 7.0) for array based detection methods at 65° C. The means and methods of the present invention preferably comprise the use of nucleic acid probes. A nucleic acid probe according to the present invention is an oligonucleotide, nucleic acid or a fragment thereof, which is substantially complementary to a specific nucleic acid sequence, “substantially complementary” refers to the ability to hybridize to the specific nucleic acid sequence under stringent conditions.

The skilled person knows means and methods to determine the levels of nucleic acids in a sample and compare them to control levels. Such methods may employ labeled nucleic acid probes according to the invention. “Labels” include fluorescent or enzymatic active labels as further defined herein below. Such methods include real-time PCR methods and microarray methods, like Affimetrix®, nanostring and the like.

The determination of the circRNAs or their level may also be detected using sequencing techniques. The skilled person is able to use sequencing techniques in connection with the present invention. Sequencing techniques include but are not limited to Maxam-Gilbert Sequencing, Sanger sequencing (chain-termination method using ddNTPs), and next generation sequencing methods, like massively parallel signature sequencing (MPSS), polony sequencing, 454 pyrosequencing, IIlumina (Solexa) sequencing, SOLiD sequencing, or ion torrent semiconductor sequencing or single molecule, real-time technology sequencing (SMRT).

The detection/determination of the circRNAs and the respective level may also employ nucleic acid amplification method alone or in combination with the sequencing and/or hybridization method. Nucleic acid amplification may be used to amplify the sequence of interest prior to detection. It may however also be used for quantifying a nucleic acid, e.g. by real-time PCR methods. Such methods are commonly known to the skilled person. Nucleic acid amplification methods for example include rolling circle amplification (such as in Liu, et al., “Rolling circle DNA synthesis: Small circular oligonucleotides as efficient templates for DNA polymerases,” J. Am. Chem. Soc. 1 18:1587-1594 (1996).), isothermal amplification (such as in Walker, et al., “Strand displacement amplification—an isothermal, in vitro DNA amplification technique,” Nucleic Acids Res. 20(7): 1691-6 (1992)), ligase chain reaction (such as in Landegren, et al., “A Ligase-Mediated Gene Detection Technique,” Science 241 : 1077-1080, 1988, or, in Wiedmann, et al., “Ligase Chain Reaction (LCR)—Overview and Applications,” PCR Methods and Applications (Cold Spring Harbor Laboratory Press, Cold Spring Harbor Laboratory, NY, 1994) pp. S51-S64.)). Nucleic-acid amplification can be accomplished by any of the various nucleic-acid amplification methods known in the art, including but not limited to the polymerase chain reaction (PCR), ligase chain reaction (LCR), transcription-based amplification system (TAS), nucleic acid sequence based amplification (NASBA), rolling circle amplification (RCA), transcription-mediated amplification (TMA), self-sustaining sequence replication (3SR) and QP amplification. It will be readily understood that the amplification of the circRNA may start with a reverse transcription of the RNA into complementary DNA (cDNA), optionally followed by amplification of the so produced cDNA.

It may be desirable to reduce or diminish non circRNA prior to the determination or the presence or level of the circRNAs. To this end RNA degrading agents may be added to the sample and/or the isolated total nucleic acids, e.g. total RNA, thereof, wherein said RNA degrading agent does not degrade circRNAs or does degrade circRNAs only at lower rates as compared to linear RNAs. One such agent is RNase R. RNase R is a 3-5′ exoribonuclease closely related to RNase II, which has been shown to be involved in selective mRNA degradation, particularly of non stop mRNAs in bacteria (see Cheng; Deutscher, M P et al. (2005). “An important role for RNase R in mRNA decay”. Molecular Cell 17(2):313-318; and Venkataraman, K; Guja, K E; Garcia-Diaz, M; arzai, A W (2014). “Non-stop mRNA decay: a special attribute of trans-translation mediated ribosome rescue.”; Frontiers in microbiology 5:93. Suzuki H I, Zuo Y, Wang J, Zhang M Q, Malhotra A, Mayeda A; Characterization of RNase R-digested cellular RNA source that consists of lariat and circular RNAs from pre-mRNA splicing; Nucleic Acids Res. 2006 May 8; 34(8):e63.). RNase R has homologues in many other organisms. When a part of another larger protein has a domain that is very similar to RNase R, this is called an RNase R domain. Hence, in a preferred embodiment the sample is treated with RNase R before determination of the circRNA to deplete linear RNA isoforms from the total RNA preparation and thereby increase detection sensitivity. As outlined herein, the diagnostic or prognostic value of a single circRNA may not be sufficient in order to allow a diagnosis or prognosis with a reliable result. In such case it may be desirable to determine the presence or level of more than one circRNA in the sample and optionally comparing them to the respective control level. The skilled person will acknowledge that these more than one circRNAs may be chosen from a predetermined panel of circRNAs. Such panel usually includes the minimum number of circRNAs necessary to allow a reliable diagnosis or prognosis. The number of circRNAs of the panel may vary depending on the desired reliability and/or the prognostic or diagnostic value of the included circRNAs, e.g. when determined alone. Hence, the method according to the present invention in a preferred embodiment determines more than one circRNA from a panel of circRNAs, e.g. their presence or absence, or level, respectively.

The panel for obtaining the desired may be chosen according to the needs. In particular the skilled person may apply statistical approaches as outlined herein in order to validate the diagnostic and/or prognostic significance of a certain panel. The inventors have herein shown for a neurodegenerative disease the development of a certain panel of circRNAs giving a reasonable degree of certainty. The skilled person may apply common statistical techniques in order to develop a panel of circRNAs. Such statistical techniques include cluster analysis (e.g. hierarchical or k-means clustering), principle component analysis or factor analysis.

In principle, the statistical methods aim the identification of circRNAs or panels of circRNAs that exhibit differing presence and/or levels in samples of diseased and healthy/normal subjects. As outlined, the panel is preferably a panel of more than one circRNA, i.e. a plurality. In a preferred embodiment of the invention said panel comprises a plurality of circRNAs that have been identified as being present at differing levels in bodily fluid samples of patients having the disease and patients not having the disease. The panel of circRNAs has been preferably identified by principle component analysis or clustering. The “principle component analysis” (PCA) (as also used exemplified herein) regards the analysis of factors differing between diseased and healthy subjects. PCA is known to the skilled person (see Pearson K., “On lines and planes of closest fit to systems of points in space”, The London, Edinburgh, and Dublin Philosophical Magazine and Journal of Science 2.1 1 559-572 (1901), and Hotelling H., “Analysis of a complex of statistical variables into principal components” Journal of educational psychology 24.6 417 (1933)). The circRNAs to be chosen for the principle component analysis may be those previously determined in samples of healthy and/or diseased subject. Thresholds may be incorporated in order to consider a circRNA for further analysis, in a preferred embodiment only circRNAs having an expression value of at least 6.7 after variance stabilizing transformation of raw read counts in one of the samples. PCA may be performed on circRNAs included in the analysis using the prcomp function of the standard package “stats” of the “R” programming language (R Core Team (2013). R: A language and environment for statistical computing. R Foundation for Statistical Computing, Vienna, Austria. ISBN 3-900051-07-0). Depending on the circRNAs chosen, the disease and other factors, the weights can vary. However, the skilled artisan will acknowledge that these circRNAs with the highest weight as regards the principle component of interest, i.e. disease/healthy state, shall be chosen in order to obtain the circRNAs with the highest predictive absolute values. PCA is used to visualize and measure the amount of variation in a data set. Mathematically, PCA is an orthogonal linear transformation that transforms the data to a new coordinate system such that the greatest variance by some projection of the data lies on the first coordinate which is called Principal Component 1 (PCI) and so on. The before mentioned calculated weight represents the distance of each circular RNA to this specific projection. Thus, the higher the absolute value the more relevant is for this projection.

“Hierarchical Clustering” (also referred to herein as “clustering”) may be performed as known in the art (reviewed in Murtagh, F and Conteras, P “Methods of hierarchical clustering” arXiv preprint arXiv: 1105.0121 (201 1)). Samples may be clustered on log2 transformed normalized circRNA expression profiles (log2(ni+1)). Hierarchical, agglomerative clustering may be performed with complete linkage and optionally by further using Spearman's rank correlation as distance metric (1−{corr [log(n; +1)]}). “The goal of cluster analysis is to partition observations (here circRNA expression) into groups (“clusters”) so that the pairwise dissimilarities between those assigned to the same cluster tend to be smaller than those in different clusters” (see Friedman J, Hastie T, and Tibshiriani R, “The elements of statistical learning”, Vol. 1. Soringer, Berlin: Springer series in statistics (2001)). Here, the measure for dissimilarity is defined as the Spearman's rank correlation. A visualization and complete description of the hierarchical clustering is provided by a dendrogram.

As disclosed herein, one or more circRNAs useful in the methods of the present disclosure include but are not limited to circHOMER1, circDOCK1, circKCNN2, circMAN2A1, circST18, circATRNL1, circEXOSC1, circICA1, circFMN1, circRTN4, circCDR1-AS, circMAP7, circTTLL7, circFANCL, circEPB41L5, circCORO1C, circDGKI, circKATNAL2, circWDR78, circADGRB3, circPLEKHM3, circERBIN, circPICALM, circRNASEH2B, circPDE4B, circPHC3, circFAT3, circMLIP, circLPAR1, circSLAIN2, circSPHKAP, circYY1AP1, circDNAJC6, circVCAN, circCDYL, circCUL5, circSOBP, circDGKB, circADARB1, circMVB12B, circNDST3, circNEBL, circRIMS1, circNELL1, circSPPL3, circZNF91, circPAN3, circUBAP2, circLINC00869, circAC011995.1, circFTO, circDOCK4, circRP1-73A14.1, circHNRNPM, circST6GAL2, circQKI, circVAPB, circSLC39A10, circCSMD3, circDOCK5, circPREX1, circZC3H6, circC16orf62, circRP5-1115A15.2, circUNC13C, circHP1BP3, circUBE3A, circSLC8A3, circBAZ2B, circSLC8A1, circMAN1A2, circCOL4A5, circZBED3-AS1, circREPS1, circPDE8A, circRNA5SP88, circAGTPBP1, circTOP1, circL3MBTL4, circRP11-406A9.2, circMEIS3, circBPTF, circDGKH, circPROSC, circEGLN3, circFANCB, circSLC30A7, circURI1, circREPS2, circRP11-1023L17.1, circSLAIN1, circTASP1, circATP8A1, circSV2B, circUSP54, circEDIL3, circLRRC49, circPSEN1, circAMD1, circLY86-AS1, circBMPER, circPTK2, circARHGEF26, circCCDC66, circAP3B1, circATG4C, circCCSER1, circKDM8, circRSRC1, circKCNH8, circRAPGEF5, circRHBDD1, circGRHPR, circZNF430, circHAT1, circSLCO1A2, circCACNA2D1, circCADPS, circFOXN2, circCEP85L, circRBBP8, circZNF148, circNOL4, circKLHDC1, circPHIP, circSPAST, circFRYL, circNRCAM, circMAPK4, circPEX5L-AS1, circSLC4A10, circDYNC1H1, circLPXN, circPCNX2, circVRK2, circFCHO2 ,circC10orf90, circKHDRBS3, circLZIC, circMBTD1, circSFMBT2, circDEK, circSLC45A4, circRNF19B, circRNF10, circNFKB1, circCDC14B, circELK4, circPLD1, circDNAJC3, circFGF14, circSH3GL3, circTMEM132D, circLRBA, circARPP21, circAAGAB, circSSX2IP, circFBXL17, circAPPL1, circUBXN2A, circGRIN2B, circAUTS2, circMBOAT2, circPGAP1, circCADPS2, circSENP6, circSIRT5, circASXL1, circTRAPPC12, circATE1, circRIMS2, circSORBS1, circMAML2, circIQCK, circMTUS1, circPTPN13, circFBXO7, circTERF2, circMED13, circLRRC7, circKLHL8, circHDAC9, circLIFR, circEML2, circFER, circCUX1, circLMTK2, circATXN7, circEP300, circTBC1D12, circWDR49 circELMOD3, circC1orf27, circNRXN1, circREXO4, circFSIP1, circSTXBP5L, circNARS2, circRNF13, circFAM208A, circHIPK3, circARHGAP32, circC2CD5, circKIAA0368, circCHD9, circRICTOR, circERI3, circARHGAP12, circDENND1A, circMARCH6, circRHOBTB3, circXYLT1, circPRUNE2, circPPP4R1, circAASS, circKCNMA1, circGPSM2, circGRM8, circPXK, circATP6V1H, circUBE2D2, circANAPC2, circMDGA2, circVPS37C, circNFASC, circPTPRR, circHDAC5, circUSP31, circGAB1, circANKIB1, circPTPRN2, circAP000304.12, circPLPPR5, circPRKAR1B, circHNRNPC, circKIF6, circUSP30, circCTNNA3, circMKLN1, circEML6, circSCLT1, circFAM120A, circZNF365, circZNF609, circATM, circARHGAP5, circIQGAP1, circBARD1, circNSD1, circRNF169, circHMBOX1, circFCHSD2, circRPS6KA5, circTARBP1, circOXCT1, circBIRC6, circFGD4, circNPAS3, circCREBRF, circEIF4G3, circGLIS2, circDUTP7, circCNOT2, circVWA3A, circOSBPL1A, circARFGEF2, circCASP8AP2, circRP11-191L9.4, circSUZ12, circTUSC3, circFAM135A, circZNF292, circDDI2, circPSMB1, circEXOC6B, circNOX4, circPDE8A, circSOS2, circRERE, circRGS7, circCLIP4, circRELL1, circSNTG1, circCNTNAP5, circZNF652, circHIPK3, circDMD, circBBX, circZKSCAN1, circRBM39, circARAP2, circZC3H13 , circCSMD1, circMETTL6, circSAMD4A, circCAP2, circATXN10, circARHGAP26, circRP11-223P11.3, circLPAR1, circNSD1, circGPBP1L1, circNEURL1, circSSH2, circEMB the RNA sequence for each is known and publically available, for example, in public databases such as Ensembl, UniProt and Entrez gene.

The one or more circRNAs disclosed herein encompass characteristic profiles which are identified as differentially expressed in a biological sample obtained from a subject relative to a reference value. See, e.g., the Examples below. In various embodiments, determining one or more circRNAs expression levels can be supplemented with diagnostic assays such as assays to determine presence, absence, amyloid plaques, advanced radiographic assays, and diagnostic assays.

In some embodiments, the methods may comprise determining the expression level of at least 1 circRNA, at least 2 circRNAs, at least 3 circRNAs, at least 4 circRNAs, at least 5 circRNAs, at least 6 circRNAs, at least 6 circRNAs, at least 7 circRNAs, at least 8 circRNAs, at least 9 circRNAs, at least 10 circRNAs, at least 11 circRNAs, at least 12 circRNAs, at least 13 circRNAs, at least 14 circRNAs, at least 15 circRNAs, at least 16 circRNAs, at least 17 circRNAs, at least 18 circRNAs, at least 19 circRNAs, at least 20 circRNAs, at least 21 circRNAs, at least 22 circRNAs, at least 23 circRNAs, at least 24 circRNAs, at least 25 circRNAs, at least 26 circRNAs, at least 27 circRNAs, at least 28 circRNAs, at least 29 circRNAs, at least 30 circRNAs, at least 31 circRNAs, at least 32 circRNAs, at least 33 circRNAs, at least 34 circRNAs, at least 35 circRNAs, at least 36 circRNAs, at least 37 circRNAs, at least 38 circRNAs, at least 39 circRNAs, at least 40 circRNAs, at least 41 circRNAs, at least 42 circRNAs, or at least 43 circRNAs, at least 44 circRNAs, at least 45 circRNAs, at least 46 circRNAs, at least 47 circRNAs, at least 48 circRNAs, at least 49 circRNAs, at least 50 circRNAs, at least 51 circRNAs, at least 52 circRNAs, at least 53 circRNAs, at least 54 circRNAs, at least 55 circRNAs, at least 56 circRNAs, at least 57 circRNAs, at least 58 circRNAs, at least 59 circRNAs, at least 60 circRNAs, at least 61 circRNAs, at least 62 circRNAs, at least 63 circRNAs, at least 64 circRNAs, at least 65 circRNAs, at least 66 circRNAs, at least 67 circRNAs, at least 68 circRNAs, at least 69 circRNAs, at least 70 circRNAs, at least 71 circRNAs, at least 72 circRNAs, at least 73 circRNAs, at least 74 circRNAs, at least 75 circRNAs, at least 76 circRNAs, at least 77 circRNAs, at least 78 circRNAs, at least 79 circRNAs, at least 80 circRNAs, at least 81 circRNAs, at least 82 circRNAs, at least 83 circRNAs, at least 84 circRNAs, at least 85 circRNAs, at least 86 circRNAs, at least 87 circRNAs, at least 88 circRNAs, at least 89 circRNAs, at least 90 circRNAs, at least 91 circRNAs, at least 92 circRNAs, at least 93 circRNAs, at least 94 circRNAs, at least 95 circRNAs, at least 96 circRNAs, at least 97 circRNAs, at least 98 circRNAs, at least 99 circRNAs, at least 100 circRNAs, at least 101 circRNAs, at least 102 circRNAs, at least 103 circRNAs, at least 104 circRNAs, at least 105 circRNAs, at least 106 circRNAs, at least 107 circRNAs, at least 108 circRNAs, at least 109 circRNAs, at least 110 circRNAs at least 111 circRNAs, at least 112 circRNAs, at least 113 circRNAs, at least 114 circRNAs, at least 115 circRNAs, at least 116 circRNAs, at least 117 circRNAs, at least 118 circRNAs, at least 119 circRNAs, at least 120 circRNAs, at least 121 circRNAs, at least 122 circRNAs, at least 123 circRNAs, at least 124 circRNAs, at least 125 circRNAs, at least 126 circRNAs, at least 127 circRNAs, at least 128 circRNAs, at least 129 circRNAs, at least 130 circRNAs, at least 131 circRNAs, at least 132 circRNAs, at least 133 circRNAs, at least 134 circRNAs, at least 135 circRNAs, at least 136 circRNAs, at least 137 circRNAs, at least 138 circRNAs, at least 139 circRNAs, at least 140 circRNAs, at least 141 circRNAs, at least 142 circRNAs, at least 143 circRNAs, at least 144 circRNAs, at least 145 circRNAs, at least 146 circRNAs, at least 147 circRNAs, at least 148 circRNAs, at least 149 circRNAs , at least 150 circRNAs, at least 151 circRNAs, at least 152 circRNAs, at least 153 circRNAs, at least 154 circRNAs, at least 155 circRNAs, at least 156 circRNAs, at least 157 circRNAs, at least 158 circRNAs, at least 159 circRNAs, at least 160 circRNAs, at least 161 circRNAs, at least 162 circRNAs, at least 163 circRNAs, at least 164 circRNAs, at least 165 circRNAs, at least 166 circRNAs, at least 167 circRNAs, at least 168 circRNAs, at least 169 circRNAs, at least 170 circRNAs, at least 171 circRNAs, at least 172 circRNAs, at least 173 circRNAs, at least 174 circRNAs, at least 175 circRNAs, at least 176 circRNAs, at least 177 circRNAs, at least 178 circRNAs, at least 179 circRNAs, at least 180 circRNAs, at least 181 circRNAs, at least 182 circRNAs, at least 183 circRNAs, at least 184 circRNAs, at least 185 circRNAs, at least 186 circRNAs, at least 187 circRNAs, at least 188 circRNAs, at least 189 circRNAs, at least 190 circRNAs, at least 191 circRNAs, at least 192 circRNAs, at least 193 circRNAs, at least 194 circRNAs, at least 195 circRNAs, at least 196 circRNAs, at least 197 circRNAs, at least 198 circRNAs, at least 199 circRNAs, at least 200, at least 250, at least 300 or at least 350 circRNAs.

The inventors have exemplified the method outlined above for a neurodegenerative disease, in particular Alzheimer's disease. A neurodegenerative disease in context with the present invention is to be understood as a disease associated with neurodegeneration. Neurodegeneration means a progressive loss of structure or function of neurons, including death of neurons. Many neurodegenerative diseases including ALS, Parkinson's, Alzheimer's, and Huntington's occur as a result of neurodegenerative processes. Nowadays, many similarities exist that relate these diseases to one another on a sub-cellular level. There are many parallels between different neurodegenerative disorders including atypical protein assemblies (protein misfolding and/or agglomeration) as well as induced cell death. Neurodegeneration can be found in many different levels of neuronal circuitry ranging from molecular to systemic. Hence, in a preferred embodiment of the present invention the disease is a neurodegenerative disease, preferably selected from the group of Alzheimer's, ALS, Parkinson's, and Huntington's. In a particularly preferred embodiment the disease is Alzheimer's disease. Alzheimer's disease has been identified as a protein misfolding disease (proteopathy), causing plaque accumulation of abnormally folded amyloid beta protein, and tau protein in the brain. Plaques are made up of small peptides, 39-43 amino acids in length, called amyloid beta (Aβ). Aβ is a fragment from the larger amyloid precursor protein (APP). APP is a transmembrane protein that penetrates through the neuron's membrane. APP is critical to neuron growth, survival, and post-injury repair. In Alzheimer's disease, an unknown enzyme in a proteolytic process causes APP to be divided into smaller fragments. One of these fragments gives rise to fibrils of amyloid beta, which then form clumps that deposit outside neurons in dense formations known as senile plaques. AD is also considered a tauopathy due to abnormal aggregation of the tau protein. In AD, tau undergoes chemical changes, becoming hyperphosphorylated; it then begins to pair with other threads, creating neurofibrillary tangles and disintegrating the neuron's transport system. A patient, is classified as having Alzheimer's disease according to the criteria as set by the National Institute of Neurological and Communicative Disorders and Stroke (NINCDS) and the Alzheimer's disease and Related Disorders Association (ADRDA, now known as the Alzheimer's Association), the NINCDS-ADRDA Alzheimer's Criteria for diagnosis in 1984, extensively updated in 2007 (see McKhann G, Drachman D, Folstein M, et al. Clinical Diagnosis of Alzheimer's disease: Report of the NINCDS-ADRDA Work Group under the Auspices of Department of Health and Human Services Task Force on Alzheimer's disease. Neurology. 1984; 34(7):939-44; and Dubois B, Feldman HH, Jacova C, et al. Research Criteria for the Diagnosis of Alzheimer's disease: Revising the NINCDS-ADRDA Criteria. Lancet Neurology. 2007; 6(8):734-469). These criteria require that the presence of cognitive impairment, and a suspected dementia syndrome, be confirmed by neuropsychological testing for a clinical diagnosis of possible or probable Alzheimer's disease. A histopathologic confirmation including a microscopic examination of brain tissue is required for a definitive diagnosis. Good statistical reliability and validity have been shown between the diagnostic criteria and definitive histopathological confirmation (see Blacker D, Albert M S, Bassett S S, et al. Reliability and validity of NINCDS-ADRDA criteria for Alzheimer's disease. The National Institute of Mental Health Genetics Initiative. Archives of Neurology. 1994; 51(12):1198-204). Eight cognitive domains are most commonly impaired in AD—memory, language, perceptual skills, attention, constructive abilities, orientation, problem solving and functional abilities. These domains are equivalent to the NENCDS-ADRDA Alzheimer's Criteria as listed in the Diagnostic and Statistical Manual of Mental Disorders (DSM-IV-TR) published by the American Psychiatric Association.

Another aspect of the present disclosure encompasses methods to diagnose subjects as pre-symptomatic or asymptomatic or having a high risk of conversion to mild cognitive impairment due to Alzheimer's disease, and to optionally stage or classify the subject in terms of the number of years of onset to MCI due to AD. Pre-symptomatic or asymptomatic refers to a subjects at risk of or having markers for AD but show no signs of cognitive impairment. Mild cognitive impairment (MCI) due to Alzheimer's disease (AD) refers to the symptomatic pre-dementia phase of AD. This degree of cognitive impairment is not normal for age and, thus, constructs such as age-associated memory impairment and age-associated cognitive decline do not apply. MCI due to AD is a clinical diagnosis, and clinical criteria for the diagnosis of MCI due to AD are known in the art. See, for instance, Albert et al. Alzheimer's & Dementia, 2011, 7(3): 270-279. Cognitive testing is optimal for objectively assessing the degree of cognitive impairment for a subject. Scores on cognitive tests for subjects with MCI are typically 1 to 1.5 standard deviations below the mean for their age and education matched peers on culturally appropriate normative data (i.e., for the impaired domain(s), when available). The designation of MCI is often supported by a global rating of 0.5 on the Clinical Dementia Rating (CDR) scale. The CDR is a numeric scale used to quantify the severity of symptoms of dementia. Other suitable cognitive tests are known in the art. While suitable tests exist to assess the severity of cognitive impairment, there is a need in the art for a test that identifies subjects with a high degree of confidence years before the onset of MCI due to AD.

In one embodiment, a method to diagnose a subject as having a high risk of conversion to MCI due to AD may comprise providing or having been provided a biological sample from a subject; detecting the expression levels of one or more circRNA in the sample; comparing the levels of one or more circRNA with a reference value; diagnosing the subject as having a high risk of conversion to MCI due to AD when the measured circRNA expression levels deviate from the reference value. In another embodiment, a method to diagnose a as an asymptomatic AD subject, the method comprising providing or having been provided a biological sample from a subject; detecting the expression levels of one or more circRNA in the sample; comparing the levels of one or more circRNA with a reference value; diagnosing the subject as having a high risk of conversion to MCI due to AD when the measured circRNA expression levels deviate from the reference value. An “asymptomatic subject” refers to a subject that does not show any signs or symptoms of AD. A subject may however exhibit signs or symptoms of AD (e.g., memory loss, misplacing things, changes in mood or behavior, etc.) but not show sufficient cognitive or functional impairment for a clinical diagnosis of mild cognitive impairment. In further embodiments, a subject may carry one of the gene mutations known to cause dominantly inherited Alzheimer's disease. In alternative embodiments, a subject may not carry a gene mutation known to cause dominantly inherited Alzheimer's disease. Alzheimer's disease that has no specific family link is referred to as sporadic Alzheimer's disease.

Another aspect of the present disclosure encompasses methods to diagnose a subject's stage of Alzheimer's disease. In various embodiments, a “stage of AD” may be defined as an amount of time (e.g. 1, 2, 3, 4, 5, 6, 7, 8, 9, 10, 11, 12 months, etc.) that has elapsed since the onset of MCI due to AD. Although there are criteria for a clinical diagnosis of AD, it is common in the clinical setting for the timing of symptom onset to be unknown for a given subject or for there to be a questionable diagnosis of either MCI or AD. As such, there is a need in the art for a test that objectively diagnoses a subject's stage of AD.

In one embodiment, a method to diagnose a subject's severity of AD may comprise providing or having been provided a biological sample from a subject; detecting the expression levels of one or more circRNA in the sample; comparing the levels of one or more circRNA with a reference value; classifying the severity of AD in the subject when the measured circRNA expression levels deviate or from the reference value of a healthy subject(s) or are the same or similar to the reference value of a subject with similar disease severity. In another aspect, the present disclosure encompasses a method to diagnose a subject as having dementia due to Alzheimer's disease (AD) comprising providing or having been provided a biological sample from a subject; detecting the expression levels of one or more circRNA in the sample; comparing the levels of one or more circRNA with a reference value; diagnosing the subject as having a dementia due to Alzheimer's disease (AD) when the measured circRNA expression levels deviate from the reference value of a healthy subject(s) or are similar to the reference value of a subject(s) with dementia.

In another aspect, present disclosure encompasses a method for enrolling a subject into a clinical trial comprising providing or having been provided a biological sample from a subject; detecting the expression levels of one or more circRNA in the sample; comparing the levels of one or more circRNA with a reference value.

In one aspect, each of the above methods may comprise detecting the level of one or more circRNA is selected from circHOMER1, circKCNN2, circMAN2A1, circICA1, circFMN1, circDOCK1, circRTN4, circST18, circIATRNL1, and circEXOSC1.

In another aspect, each of the above methods may comprise detecting the level of one or more circRNA is selected from circHOMER1, circDOCK1, circKCNN2, circMAN2A1, circST18, circATRNL1, circEXOSC1, circICA1, circFMN1, circRTN4, circCDR1-AS, circMAP7, circTTLL7, circFANCL, circEPB41L5, circCORO1C, circDGKI, circKATNAL2, circWDR78, circADGRB3, circPLEKHM3, circERBIN, circPICALM, circRNASEH2B, circPDE4B, circPHC3, circFAT3, circMLIP, circLPAR1, circSLAIN2, circSPHKAP, circYY1AP1, and circDNAJC6.

In some embodiments, the subject is classified with a disease status or disease severity when circDOCK1, circMAN2A1, circST18, circEXOSC1, circRTN4, circCDR1-AS, circMAP7, circTTLL7, circFANCL, circCORO1C, circWDR78, circERBIN, circPICALM, circRNASEH2B, circPHC3, circLPAR1, circSLAIN2, circYY1AP1, and circDNAJC6 are increased relative to a reference value and/or one or more of circHOMER1, circKCNN2, circATRNL1, circICA1, circFMN1, circEPB41L5, circDGKI, circKATNAL2, circPLEKHM3, circPDE4B, circFAT3, circMLIP, circSPHKAP, and circDNAJC6 are decreased relative to a reference value.

### III. Treatment

Another aspect of the present disclosure is a method for treating a subject in need thereof. The terms “treat,” “treating,” or “treatment” as used herein, refers to the provision of medical care by a trained and licensed professional to a subject in need thereof. The medical care may be a diagnostic test, a therapeutic treatment, and/or a prophylactic or preventative measure. The object of therapeutic and prophylactic treatments is to prevent or slow down (lessen) an undesired physiological change or disease/disorder. Beneficial or desired clinical results of therapeutic or prophylactic treatments include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition or disorder or those in which the disease, condition or disorder is to be prevented. In some embodiments, a subject receiving treatment is asymptomatic. An “asymptomatic subject,” as used herein, refers to a subject that does not show any signs or symptoms of AD. In other embodiments, a subject may exhibit signs or symptoms of AD (e.g., memory loss, misplacing things, changes in mood or behavior, etc.,) but not show sufficient cognitive or functional impairment for a clinical diagnosis of mild cognitive impairment or dementia due to Alzheimer's disease. The phrase “mild cognitive impairment due to Alzheimer's disease” is defined above. A symptomatic or an asymptomatic subject may have Aβ amyloidosis; however, prior knowledge of Aβ amyloidosis is not a requisite for treatment. In still further embodiments, a subject may be diagnosed as having AD. In any of the aforementioned embodiments, a subject may carry one of the gene mutations known to cause dominantly inherited Alzheimer's disease. In alternative embodiments, a subject may not carry a gene mutation known to cause dominantly inherited Alzheimer's disease.

In one embodiment, a method for treating a subject as described above may comprise (providing or having been provided a biological sample from a subject; detecting the expression levels of one or more circRNA in the sample; comparing the levels of one or more circRNA with a reference value; administering a pharmaceutical composition to the subject when the circRNA level(s) deviate from the reference value. In some embodiments, the pharmaceutical composition directly or indirect stabilizes or corrects the circRNA expression levels, in some embodiment the extent of change above or below the reference value may be used as criteria for treating a subject.

Many imaging agents and therapeutic agents are contemplated for, or used with, subjects at risk of developing Aβ amyloidosis or AD, subjects diagnosed as having Aβ amyloidosis, subjects diagnosed as having a tauopathy, or subjects diagnosed as having AD, target a specific pathophysiological change. For instance, Aβ targeting therapies are generally designed to decrease Aβ production, antagonize Aβ aggregation or increase brain Aβ clearance; tau targeting therapies are generally designed to alter tau phosphorylation patterns, antagonize tau aggregation, or increase NFT clearance; a variety of therapies are designed to reduce CNS inflammation or brain insulin resistance; etc.

In certain aspects, a therapeutically effective amount of a pharmaceutical composition may be administered to a subject. Administration is performed using standard effective techniques, including peripherally (i.e. not by administration into the central nervous system) or locally to the central nervous system. Peripheral administration includes but is not limited to oral, inhalation, intravenous, intraperitoneal, intra-articular, subcutaneous, pulmonary, transdermal, intramuscular, intranasal, buccal, sublingual, or suppository administration. Local administration, includes but is not limited to via a lumbar, intraventricular or intraparenchymal catheter or using a surgically implanted controlled release formulation. The route of administration may be dictated by the disease or condition to be treated.

Pharmaceutical compositions for effective administration are deliberately designed to be appropriate for the selected mode of administration, and pharmaceutically acceptable excipients such as compatible dispersing agents, buffers, surfactants, preservatives, solubilizing agents, isotonicity agents, stabilizing agents, and the like are used as appropriate. Remington's Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners.

In each of the above embodiments, a pharmaceutical composition may comprise an imaging agent. Non-limiting examples of imaging agents include functional imaging agents (e.g. fluorodeoxyglucose, etc.) and molecular imaging agents (e.g., Pittsburgh compound B, florbetaben, florbetapir, flutemetamol, radionuclide-labeled antibodies, etc.).

Alternatively, a pharmaceutical composition may comprise an active pharmaceutical ingredient. Non-limiting examples of active pharmaceutical ingredients include cholinesterase inhibitors, N-methyl D-aspartate (NMDA) antagonists, antidepressants (e.g., selective serotonin reuptake inhibitors, atypical antidepressants, aminoketones, selective serotonin and norepinephrine reuptake inhibitors, tricyclic antidepressants, etc.), gamma-secretase inhibitors, beta-secretase inhibitors, anti-Aβ antibodies (including antigen-binding fragments, variants, or derivatives thereof), anti-tau antibodies (including antigen-binding fragments, variants, or derivatives thereof), stem cells, dietary supplements (e.g. lithium water, omega-3 fatty acids with lipoic acid, long chain triglycerides, genistein, resveratrol, curcumin, and grape seed extract, etc.), antagonists of the serotonin receptor 6, p38alpha MAPK inhibitors, recombinant granulocyte macrophage colony-stimulating factor, passive immunotherapies, active vaccines (e.g. CAD106, AF20513, etc.), tau protein aggregation inhibitors (e.g. TRx0237, methylthionimium chloride, etc.), therapies to improve blood sugar control (e.g., insulin, exenatide, liraglutide pioglitazone, etc.), anti-inflammatory agents, phosphodiesterase 9A inhibitors, sigma-1 receptor agonists, kinase inhibitors, phosphatase activators, phosphatase inhibitors, angiotensin receptor blockers, CB1 and/or CB2 endocannabinoid receptor partial agonists, β-2 adrenergic receptor agonists, nicotinic acetylcholine receptor agonists, 5-HT2A inverse agonists, alpha-2c adrenergic receptor antagonists, 5-HT 1A and 1D receptor agonists, Glutaminyl-peptide cyclotransferase inhibitors, selective inhibitors of APP production, monoamine oxidase B inhibitors, glutamate receptor antagonists, AMPA receptor agonists, nerve growth factor stimulants, HMG-CoA reductase inhibitors, neurotrophic agents, muscarinic M1 receptor agonists, GABA receptor modulators, PPAR-gamma agonists, microtubule protein modulators, calcium channel blockers, antihypertensive agents, statins, and any combination thereof.

In some embodiments, a minimal dose is administered, and dose is escalated in the absence of dose-limiting toxicity. Determination and adjustment of a therapeutically effective dose, as well as evaluation of when and how to make such adjustments, are known to those of ordinary skill in the art of medicine.

The frequency of dosing may be daily or once, twice, three times or more per week or per month, as needed as to effectively treat the symptoms. The timing of administration of the treatment relative to the disease itself and duration of treatment will be determined by the circumstances surrounding the case. Treatment could begin immediately, such as at the site of the injury as administered by emergency medical personnel. Treatment could begin in a hospital or clinic itself, or at a later time after discharge from the hospital or after being seen in an outpatient clinic. Duration of treatment could range from a single dose administered on a one-time basis to a life-long course of therapeutic treatments.

Typical dosage levels can be determined and optimized using standard clinical techniques and will be dependent on the mode of administration.

A subject may be a rodent, a human, a livestock animal, a companion animal, or a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas, and alpacas. In still another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a preferred embodiment, the subject is a human.

Additionally, a subject in need thereof may be a subject suffering from, suspected of suffering from or at risk of pharyngitis.

### IV. Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to systems, assays, primers, or software. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or from a randomized group of subjects. A reference value can be used in place of a control or reference sample, which was previously obtained from a healthy subject or a group of healthy subject. A control sample or a reference sample can also be a sample with a known amount of a detectable compound or a spiked sample.

The methods and algorithms of the invention may be enclosed in a controller or processor. Furthermore, methods and algorithms of the present invention, can be embodied as a computer implemented method or methods for performing such computer-implemented method or methods, and can also be embodied in the form of a tangible or non-transitory computer readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer program include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

As various changes could be made in the above-described materials and methods without departing from the scope of the invention, it is intended that all matter contained in the above description and in the examples given below, shall be interpreted as illustrative and not in a limiting sense.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### An Atlas of Cortical Circular RNA Expression in Alzheimer Disease Brains Demonstrates Clinical and Pathological Associations

Transcriptional regulation underlies the complexity of the human nervous system, and its misregulation can contribute to disease (Lee, T. I., et al., Cell 152, 1237-1251 (2013)). Indeed, several studies focused on the linear transcriptome have identified co-expression networks and changes in splicing associated with AD status (Zhang, B. et al., Cell 153, 707-720 (2013); Karch C. M. Et al., PloS One &, e50976 (2012); Raj, T. et al., Nat. Genet. 50, 1584 (2018); Vereijen, J. et al., Trends Genet. TIG 34, 434-447 (2018)). The present Example provides insight into the AD-associated circular transcriptome. Parietal cortex RNA-sequencing (RNA-seq) data were generated from individuals with and without Alzheimer disease (AD; n_{control}=13; n_{AD}=83) from the Knight Alzheimer Disease Research Center (Knight ADRC). Using this and an independent (Mount Sinai Brain Bank (MSBB)) AD RNA-seq dataset, cortical circular RNA (circRNA) expression was quantified in the context of AD. Significant associations were identified between circRNA expression and AD diagnosis, clinical dementia severity and neuropathological severity. It was demonstrated that most circRNA-AD associations are independent of changes in cognate linear messenger RNA expression or estimated brain cell-type proportions. Evidence was provided for circRNA expression changes occurring early in presymptomatic AD and in autosomal dominant AD. It was also observed that AD-associated circRNAs co-expressed with known AD genes. Finally, potential microRNA-binding sites were identified in AD-associated circRNAs for miRNAs predicted to target AD genes. Together, these results highlight the importance of analyzing non-linear RNAs and support future studies exploring the potential roles of circRNAs in AD pathogenesis.

### Methods

Code Availability. A description of how all software has been run for this Example, including relevant command flags, is included in the Methods and the code used for analysis is provided in Dube, U., Del-Aguila, J. L., Li, Z. et al. An atlas of cortical circular RNA expression in Alzheimer disease brains demonstrates clinical and pathological associations. Nat Neurosci 22, 1903-1912 (2019), which is incorporated herein by reference.

### RNA-Sequencing

Discovery (Knight ADRC) and Autosomal Dominant AD (DIAN) datasets. 151 nucleotide (nt), paired-end, rRNA depleted RNA-sequencing (RNA-seq) data were generated from frozen brain parietal cortex tissue. The frozen brain tissues were donated by participants in either the prospective Knight Alzheimer's Disease Research Center (Knight ADRC) Memory and Aging Project study at Washington University School of Medicine or the Dominantly Inherited Alzheimer's Network (DIAN) study. All participants consented to brain donation and neuropathological analysis. the frozen cortical tissues was first disrupted using a TissueLyser LT and purified the RNA from this disrupted tissue using RNeasy Mini Kits. (Qiagen, Hilden, Germany). The RNA Integrity Number (RIN) was calculated using a RNA 6000 Pico assay on a Bioanalyzer 2100 (Agilent Technologies, Santa Clara, USA). The extracted RNA was also quantified using the Quant-iT RNA assay (Invitrogen, Carlsbad, USA) on a Qubit Fluorometer (Fisher Scientific, Waltham, USA). Prior to library construction, External RNA Controls Consortium (ERCC) (Pine, P. S. et al., BMC Biotechnol. 16, 54 (2016)) RNA Spike-In Mix (Invitrogen, Carlsbad, USA) was introduced. rRNA depleted cDNA libraries were prepared using a TruSeq Stranded Total RNA Sample Prep with Ribo-Zero Gold kit (Illumina, San Diego, USA) and sequenced on an Illumina HiSeq 4000 at the McDonnell Genome Institute at Washington University in St. Louis. All samples were randomly assigned to a sequencing pool prior to sequencing and RNA extraction and sequencing library preparation were performed blind to neuropathological case-control status. The average number of raw sequencing reads per individual was 58,094,683, see e.g., Umber, D., et al., Nat. Neurosci. 22(110); 1903-1912 (2019), Supplemental Table 6, incorporated herein by reference.

Replication Dataset (MSBB). Publicly available RNA-seq data from the Synapse portal (syn3157743, accessed May 2018) were downloaded from the Advanced Medicine Partnership for AD: Mount Sinai Brain Bank (MSBB) dataset. In short, this dataset was generated by sequencing RNA derived from four different cortical regions: frontal pole (Brodmann area (BM) 10), superior temporal gyrus (BM22), parahippocampal gyrus (BM36) and inferior frontal gyrus tissue (BM44) from 301 individuals. rRNAs was depleted using the Ribo-Zero rRNA Removal Kit (Human/Mouse/Rat) (Illumina, San Diego, USA). Sequencing libraries were prepared using TruSeq RNA Sample Preparation kit v2. From these libraries, rRNA-depleted 101nt single-end, and non-stranded RNA-seq data was generated via an Illumina HiSeq 2500 (Illumina, San Diego, USA) (Wang, M. et al., Sci Data 5, 180185 (2018)). The average number of raw sequencing reads per individual was 35,062,514.

Alzheimer Disease Traits. In this Example, differential expression and correlation of circular RNA (circRNA) expression was investigated in human cortical tissues with Alzheimer Disease (AD) case-control status, autosomal dominant Alzheimer Disease (ADAD) case-control status, and two AD quantitative traits: clinical dementia rating at expiration/death (CDR) and Braak score.

Case-control status was determined by post-mortem, neuropathological analysis of study participant brains following CERAD (Pamudurti, N.R. et al., Mol. Cell 66, 9-21.e7 (2017)) and/or Khachaturian (Panda, A.c. et al., Nucleic Acids Res. Doi:10.1093/nar/gkx297) criteria. ADAD status was determined via pre-mortem sequencing of APP, PSEN1, and PSEN2 genes to identify established, pathogenic mutations (Cheng, J. et al., Bioinforma. Oxf. Engl. 32, 1094-1096 (2016)). CDR is a clinical measure of cognitive impairment with a range from 0 (no dementia) to 3 (severe dementia) Rossetti, H. C. et al., Alzheimer Dis. Assoc. Disord. 24, 138-142 (2010)). Braak score is a neuropathological measure of AD severity, as determined by the number and distribution of neurofibrillary tau tangles through the brain (Li, Y. et al., Cell Res. 25, 981-984 (2015)). Braak scores range from 0 (absent, at most incidental tau tangles) to 6 (severe, extensive tau tangles in neocortical areas). Importantly, the neuropathological diagnoses available are based on criteria that require the presence of “neuritic” or “senile” plaques and thus individuals with neurofibrillary tau tangles but without plaques may still be considered controls. A subset of the AD brains were identified that were from individuals with pre-symptomatic or pre-clinical AD. These individuals did not have clinically significant dementia (clinical dementia rating<=0.5, at most, very mild dementia) but their brains had evidence of AD neuropathological changes. Finally, the MSBB dataset included an additional AD neuropathological quantitative trait, mean amyloid plaque number.

### Phenotype Processing

Discovery Dataset: Knight ADRC. Genetic ancestry covariates were generated using principal components analysis via PLINK v.1.9 software (Chang, C.C. et al., GigaScience 4, 7 (2015)) and previously generated Genome-wide Association Study (GWAS) data. In brief, genetic microarray data from the Knight ADRC study participants were merged with the HapMap reference panel (Gibbs, R.A. et al., Nature 426, 789-796 (2003)), filtered to include only variants with a mean allele frequency >5% and a genotype rate >95%, pruned to include only those variants that were not in linkage disequilibrium, and the -pca command was used. The first two principal components were used to represent genetic ancestry for downstream analyses. Differential expression, correlation and meta-analyses were only conducted using parietal cortex-derived samples donated by individuals from whom all differential expression analysis covariates (PMI, median TIN32, a measure of RNA quality, AOD, batch, sex and genetic ancestry covariates) were available.

Samples from individuals who were neuropathologically classified as controls but had mild or worse dementia (CDR≤1), that is demented controls, were excluded because their dementias can be expected to have non-AD etiologies.

Four samples were excluded because their circular transcriptomic profiles, as measured by the first two transcriptomic principal components, were outliers compared with the distribution of other parietal region samples.

Replication Dataset: MSBB. Additional data were downloaded from the MSBB replication dataset, including clinical phenotype and RNA-seq covariates (syn12178045), whole genome sequencing (WGS) data (syn10901600) and quality control remapping data (syn12178047) from the Synapse portal (accessed May 2018). These data were processed as follows:

AOD listed as ‘90+’ was reassigned as ‘90’ to make the variable quantitative.

PMI was adjusted from minutes to hours to match the discovery dataset scale.

The number of APOE4 alleles was inferred using the WGS data based on the SNP rs429358. After confirming that there was a high concordance between the non-missing number of APOE4 alleles provided in the clinical covariates file and this inferred number, the inferred number of alleles was used for all downstream analyses to increase the number of individuals with these data.

Genetic ancestry covariates were generated from the MSBB WGS data through principal components analysis via PLINK v.1.9 software, as with the discovery dataset.

Missing batch and RIN information was assigned to files that had been resequenced using information from the original sequencing run, matching the two files on the basis of a common barcode.

Between the originally sequenced and resequenced samples, the RNA-seq data with a greater number of mapped reads were selected.

Individuals and reassigned sample-swap IDs were excluded on the basis of information provided in the quality control remapping data (syn12178047) file.

Samples were excluded from individuals who were neuropathologically classified as controls but had mild or worse dementia (CDR≤1), that is demented controls, because their dementias can be expected to have non-AD etiologies.

Five samples were excluded because their circular transcriptomic profiles, as measured by the first two transcriptomic principal components, were outliers compared with the distribution of other samples from that same cortical region.

Differential expression, correlation and meta-analyses were only conducted using data derived from individuals for whom information for all differential expression analysis covariates (PMI, median TIN, AOD, batch, sex and genetic ancestry covariates) were available.

RNA-seq Data Processing and Alignment. To increase detection power, RNA-seq data derived from all available samples in each dataset, not just those from samples that met inclusion criteria for downstream analyses, were processed and aligned. All RNA-seq data processing and alignment were performed blind to neuropathological case-control status.

Raw sequencing reads from the discovery RNA-seq dataset were aligned to the primary assembly of the human reference genome, GRCh38, using STAR v.2.5.3a (Dobin A. et al., Bioinforma. Oxf. Engl. 29, 15-21 (2013)) in chimeric alignment mode and parameters suggested by the documentation of the circRNA calling software, DCC (Cheng, J. et al., Bioinforma. Oxf. Engl. 32, 1094-1096 (2016)). First an alignment index was prepared with a splice junction database overhang of 150 (--sjdbOverhang 150) using the GENCODE v.26 (Harrow, J. et al., Genome Res. 22, 1760-1774 (2012)) comprehensive gene annotation. Then each mate pair was aligned individually and together, for a total of three alignments per sample, using the following parameters:

--outSJfilterOverhangMin 15 15 15 15

--alignSJoverhangMin 15

--alignSJDBoverhangMin 15

--seedSearchStartLmax 30

--outFilterMultimapNmax 20

--outFilterScoreMin 1

--outFilterMatchNmin 1

--outFilterMismatchNmax 2

--chimSegmentMin 15

--chimScoreMin 15

--chimScoreSeparation 10

--chimJunctionOverhangMin 15

The replication MSBB RNA-seq dataset was provided as aligned and unmapped files and thus required additional processing prior to alignment. After downloading aligned and unmapped files for each sample from the Synapse web portal (syn3157743), the functions of Picard tools' RevertSam, FastqToSam and MergeSamFiles were used to generate raw, unaligned files. These generated files were aligned as above using STAR v.2.5.3a, but with an alignment index suitable for 101-nt reads (--sjdbOverhang 100) and only once per sample due to the single-ended nature of the data. For all alignments, any adapter sequence was soft-clipped from the reads based on the generic IIlumina adapter sequence.

Calling circRNA-defining backsplices. DCC software v.0.4.4 (Cheng, J. et al., Bioinforma. Oxf. Engl. 32, 1094-1096 (2016)) was used to detect, annotate, quantify, filter and call circRNA-defining backsplices from the chimeric junctions identified during STAR alignment. Additional filtering was performed as reccomended in the DCC software documentation: backsplice junctions were excluded if they were located in repetitive regions of the genome (as defined in the UCSC Genome Browser: RepeatMasker and Simple Repeats tables), spanned multiple gene annotations or mapped to the mitochondrial chromosome. When analyzing paired-end data, DCC software takes into account chimeric junctions identified in both mates individually and together to improve sensitivity.

For the discovery dataset, DCC was run in paired-end, stranded mode using the following parameters:

-D -R GRCh38_Repeats_simpleRepeats_RepeatMasker.gtf -an gencode.v26.primary_assembly.annotation.gtf -Pi -F -M -Nr 1 1 -fg -G -A GRCh38.primary_assembly.genome.fa

For the replication dataset, we ran DCC in single-end, non-stranded mode with the following parameters:

-D -N -R GRCh38_Repeats_simpleRepeats_RepeatMasker.gtf -an gencode.v26.primary_assembly.annotation.gtf -F -M -Nr 1 1 -fg -G -A GRCh38.primary_assembly.genome.fa

Backsplices were also called using an additional software package, circRNA_finder (Barrett, S. P. et al., Development 143, 1838-1847 (2016), incorporated herein by reference). There was an average Pearson's correlation of 0.99 between the counts called by the DCC and circRNA_finder softwares. Similar to DCC, circRNA_finder calls backsplices from the chimeric junctions identified via STAR, but does not have parameters to adjust for type of RNA-seq data. Due to this limitation, the DCC-called backsplices were retained for downstream analyses. Backsplice calling was performed blind to neuropathological case-control status.

Filtering and collapsing annotated backsplices to identify high-confidence circRNAs. CircRNAs are detected in RNA-seq data by calling backsplices from reads overlapping chimeric junctions. Such junctions can form artifactually during library preparation via a template-switching process (Tang, C. et al., bioRxiv 259556 (2018) doi:10.1101/259556). As these artifactual junctions are formed randomly, filtering called backsplices by the number of samples in which they are observed, as well as the minimum ratio of linearly aligning versus chimerically aligning reads (circ:linear ratio), at each backsplice junction, allows for the selection of a high-confidence set of backsplices. To empirically determine the number of samples and circ:linear ratio filtering thresholds, artifactual backsplices identified in spiked-in linear ERCC (Pine, P. S. et al., BMC Biotechnol. 16, 54 (2016)) RNAs were called from the discovery dataset. As these spiked-in RNAs are linear, backsplices identified in ERCC sequences are expected to arise artifactually during the library preparation. As before, the raw sequencing reads were aligned using STAR v.2.5.3a with the same parameters as the discovery dataset, but using the ERCC92 fasta and gtf files (Invitrogen) rather than the human reference genome files. DCC was also used in stranded, paired-end mode, but without filtering for human genome annotations to identify the artifactual junctions. As expected, it was possible to detect artifactual backsplices in the ERCC spike-in RNA (FIG. 15). Based on these data, a highly conservative threshold of being observed in at least three samples with a minimum circ:linear ratio of 0.1 was selected for the inclusion of backsplices in downstream analyses.

In the discovery parietal cortex dataset, most (5,090/7,450) of the backsplice junctions identified using this calling and filtering approach were previously identified using a different calling algorithm in an independent analysis of healthy parietal cortex tissue (Glazar, P. et al., RNA (2014) doi:10.1261/rna.043687.113). After high-confidence backsplice junctions were identified, each of them was collapsed on to its annotated linear gene of origin/cognate linear mRNA for downstream differential expression and correlation analyses. Backsplices without a linear gene of origin annotation were excluded. For the MSBB replication dataset circRNA calls—which are derived from non-stranded RNA-seq data—the strand and linear gene of origin annotation was updated to match that of the stranded parietal dataset, but only if the backsplice calls had the same chromosome, start and end positions.

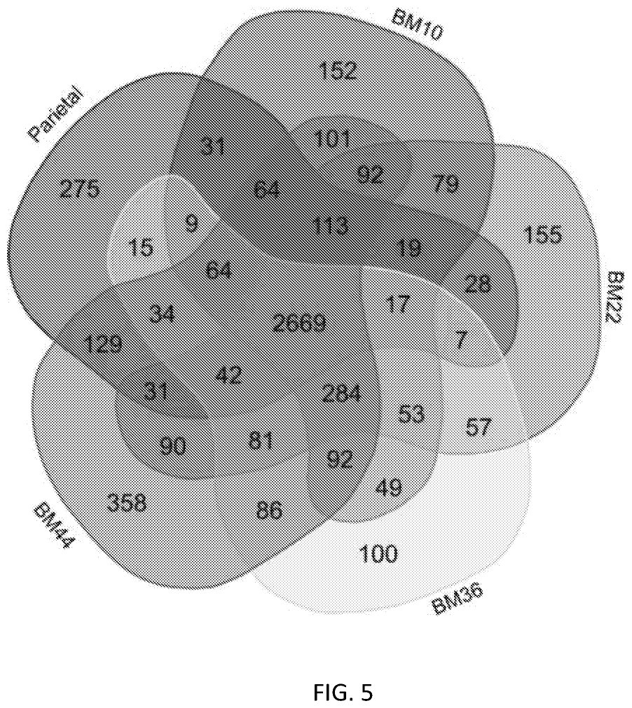

Overall, a total of 3,547, well-supported circRNAs were called in the discovery dataset and 4,330 in the larger replication dataset. There were 3,146 well-supported circRNAs common to both the discovery and the replication datasets. The overlap between the circRNAs called in each dataset was visualized using the Venn tool at: (FIG. 5). All circRNA identification was performed blind to neuropathological case-control status.

Calling linear transcripts. Linear transcripts were called using Salmon software v.0.8.2 (Patro, R. et al., Nat. Methods 14, 417-419 (2017)) in quasi-mapping-based alignment mode. In short, a quasi-mapping index was generated using the primary assembly of the human reference genome, GRCh38, and the GENCODE v.26 (Harrow, J. et al., Genome Res. 22, 1760-1774 (2012)) comprehensive gene annotation. Then the linear transcript expression was quantified from the raw, unaligned RNA-seq files for both the discovery and the replication datasets using the default Salmon pipeline parameters. All linear transcript calling was performed blind to neuropathological case-control status.

Measuring Transcript Integrity Number. TIN is measure of RNA quality that is derived from RNA-seq data and directly measures the degradation of mRNA (Wang, L. et al., BMC Bioinformatics 17, 58 (2016)). The median TIN score for each sample has been demonstrated to have robust concordance with the RIN—a commonly used measure of mRNA integrity based on rRNA amounts—in multiple, independent, RNA-seq datasets. TIN was calculated in each sample using the RSeQC software v.2.6.4 (Wang, L. et al., Bioinformatics 28, 2184-2185 (2012)) to provide a consistent quality control covariate for the differential expression and correlation analyses. In brief, the median TIN for each sample in the discovery and replication datasets was calculated based on the STAR-aligned RNA-seq coverage for the representative (annotated as ‘basic’) protein-coding transcript annotations as per GENCODE v.26.

Differential expression and correlation analyses. Differential expression and correlation analyses between the sets of high-confidence, cortical circRNA counts and AD traits were performed using the negative binominal family logistic regression and two-tailed statistical Wald test capabilities of DESeq2 v.1.18.1 (Love, M. I. et al., Genome Biol. 15, 550 (2014), incorporated herein by reference). The analysis approach of the present study follows previous analyses of circRNA differential expression (You, X. et al., Nat Neurosci. 18, 603-610 (2015); Rybak-wolf, A. et al., Mol. Cell 58, 870-885 (2015)). In general, differential expression analyses assume that the background distribution of RNA expression is equivalent between samples, with observed differences being attributable to adjustable technical differences (such as sequencing depth/library size or RNA quality) or adjustable biological differences (such as sex or AOD), or finally due to biological traits of interest (such as disease status or severity). The DESeq2 analysis approach takes all these factors into account. Before performing the logistic regression and Wald test, circRNA counts for each sample were normalized on the basis of a sequencing depth/library-size-derived size factor, estimated using circRNA counts from all samples derived from the same cortical region. After this normalization, the samples were subsetted to include only those samples for which complete information—including differential expression covariate data—was available for the particular AD trait under investigation. For example, Braak score was available for only 86/96 participants in the discovery dataset, so the sample size for discovery circRNA Braak score correlation analysis was 86. All differential expression and correlation analyses with these subsets were, in general, adjusted for the following covariates: PMI, median TIN, AOD, batch, sex and genetic ancestry—represented by the first two principal components derived from genetic data. Importantly, restricting the discovery analysis to only individuals of European genetic ancestries, that is dropping the six black individuals (five AD cases and one control), yielded consistent results (effect-size Pearson's correlation for CDR-associated circRNAs in the European-only versus original discovery parietal analysis: 0.94). No adjustment for AOD was made in the analyses that included ADAD samples. ADAD is early onset (Bateman, R. J. et al., Alzheimers Res. Ther. 3, 1 (2011)) and ADAD brains were donated by individuals who had a younger AOD compared with both control and AD participants, rendering AOD collinear with status. In addition, as GWAS data to calculate genetic ancestry covariates were unavailable for ADAD samples, self-reported ethnicity was substituted for genetic ancestry covariates in all analyses that included ADAD samples. Analyses were restricted to include only samples for which complete information for all included differential expression covariates was available. A statistical significance FDR threshold of 0.05 was set and uncorrected P values are presented in the text. DESeq2 software automatically filters out circRNAs with low expression before statistical analyses.

In the discovery and ADAD datasets, this approach was used to investigate for cortical circRNAs that are significantly differentially expressed between AD versus controls and ADAD versus controls. The present Example also investigated for cortical circRNAs that are significantly differentially expressed between ADAD and AD, adjusting for neuropathological severity as measured by the Braak score. Cortical circRNAs that were significantly correlated with CDR and Braak score were also investigated in the discovery dataset samples for which data on these AD traits was available. To replicate these findings, similar analyses were performed in the MSBB datasets. BM44 was selected as the primary replication dataset, but the analyses were performed in all cortical regions separately. The present Example investigated for significant differential cortical circRNA expression between definite AD and control status, significant correlation between CDR and cortical circRNA expression, and significant correlation between Braak score and cortical circRNA expression. Finally, analyses were performed to investigate for significant correlations between circRNAs and mean number of plaques in the MSBB dataset. With the exception of invalid statistical models due to collinearity between the quality control metric and the particular AD trait under investigation, substitution of median TIN or RIN quality control metrics yielded similar differential expression and correlation results. For example, the effect-size Pearson's correlation for the 31 discovery analysis, CDR-associated circRNAs obtained after substituting RIN for TIN is 0.99 (P=5.43×10⁻²⁶).

Validating RNA-seq Counts and Direction of Effect via Quantitative PCR. Divergent primers were designed to the backsplice junctions of circHOMER1 (forward 5′-TTTGGAAGACATGAGCTCGA-3′ (SEQ ID NO:1); reverse 5′-AAGGGCTGAACCAACTCAGA-3′ (SEQ ID NO:2)), circKCNN2 (forward 5′-GACTGTCCGAGCTTGTGAAA-3′ (SEQ ID NO:3); reverse 5′-GGCCGTCCATGTGAATGTAT-3′ (SEQ ID NO:4)), circMAN2A1 (forward 5′-TGAAAGAAGACTCACGGAGGA-3′ (SEQ ID NO:5); reverse 5′-TAGCAAACGCTCCAAATGGT-3′ (SEQ ID NO:6)), circICA1 (forward 5′-TTGATGATTTGGGGAGAAGG-3′ (SEQ ID NO:7); reverse 5′-TGGATGAAGGACGTGTCTCA-3′ (SEQ ID NO:8)), circFMN1 (forward 5′-GGTGGCTATGCAGAGAAAGC-3′ (SEQ ID NO:9); reverse 5′-CAGGGAAGACCACAGCTGAG-3′ (SEQ ID NO:10)); circDOCK1 (forward 5′-AGCTGAGGGACAACAACACC-3′ (SEQ ID NO:11); reverse 5′-GGCCGTCCATGTGAATGTAT-3′ (SEQ ID NO:12)), circRTN4 (forward 5′-TGAAAGCAGCAGGAATAGGC-3′ (SEQ ID NO:13); reverse 5′-CAGGCGCCTCTTCTTAGTTG-3′ (SEQ ID NO:14)), circST18 (forward 5′-CTGGAGTTTTCTTGTGCAGTTG-3′ (SEQ ID NO:15); reverse 5′-AAACCCAAGCTTCATGCAAG-3′ (SEQ ID NO:16)), circIATRNL1 (forward 5′-GGGTATAAAGCATTGCCAGG-3′ (SEQ ID NO:17); reverse 5′-GCCTTCAATGAGCCAAGTACA-3′ (SEQ ID NO:18)), circEXOSC1 (forward 5′-CTTTGGCCAAGACAATGTCA-3′ (SEQ ID NO:19); reverse 5′-GTGTGAGATGCAGTGCCCTA-3′ (SEQ ID NO:20)); circRNA transcripts based on circRNA fasta sequences extracted via the getcircfasta.py script provided with DCC software (. Divergent primers face outwards—as opposed to inward facing, typical primers—and as a result they only produce a PCR product in the context of a backsplice junction formed via circularization of a transcript or more rarely in the context of a transcript containing a tandem exon duplication. These primers were confirmed to be divergent through in silico PCR, and the amplification efficiency of these divergent primers was confirmed empirically to be suitable for quantitative PCR (qPCR). 13 parietal cortex-derived RNA samples were selected from individuals in the discovery study (n_{control}=3, n_{PreSympAD}=3, n_{AD}=7) to generate GAPDH-normalized expression values, which were then correlated with the RNA-seq-derived counts derived from the same individuals. Complementary DNA was generated from the RNA samples using a SuperScript VILO cDNA synthesis kit (Invitrogen) following the manufacturer's recommended protocol. With this cDNA, the qPCR experiment was performed using PowerUp SYBR Green Master Mix (Applied Biosystems) on a QuantStudio 12 K Flex Real-Time PCR System. The relative expression was calculated following the standard ΔΔCₜ method. In brief, the triplicate readings of Ct were averaged for each primer pair and the average linear GAPDH Cₜ was subtracted from the average circRNA Ct to calculate ΔCₜ. Further the ΔΔCₜ of each circRNA was calculated by subtracting the average control (n=3) ΔCt for each primer from the ΔCₜ values. Finally, the relative expression was generated using the following formula: relative expression=2^{-ΔCt}.

Meta-analyses and Overlap calculations. Meta-analyses of the cortical circRNA differential expression and correlation discovery and replication results were performed using the meta-RNA-seq R package v.1.0.2. The choice was made to combine the P values of the circRNAs common to both replication and discovery results using the inverse/Stouffer's method, due to the differences in sample size between the datasets. As before, a statistical significance threshold and FDR threshold of 0.05 were set and uncorrected P values presented. The results of the meta-analyses were visualized using the CMplot R package v.3.3.1.

Overlap between meta-analysis results was visualized using the VennDiagram R package v.1.6.20 and significance of overlap calculated using the SuperExactTest R package v.1.0.0, which reports one-tailed P values.

Independence of Circular versus Cognate Linear RNA AD-Associations or AD-Associated Changes in Estimated Brain Cell-type Proportions via Regression-Based Analyses. To demonstrate the independence of circular versus their cognate linear mRNA-AD associations, library-size-normalized counts for the CDR-associated circRNAs and their cognate linear mRNAs were included in the same regression models predicting CDR. The regression models also included the differential expression covariates: PMI, median TIN, AOD, batch, sex and genetic ancestry. Given the fact that circRNA expression levels are lower than their cognate linear mRNA expression levels, and the fact that most RNA-seq reads covering a circRNA will not include the backsplice (thereby inflating the cognate linear mRNA counts), circRNAs were considered to demonstrate an independent association with CDR if they retained a significant (P<0.05) association in the combined regression model. These regression analyses were performed for the CDR-associated circRNAs in both the discovery and the replication datasets and the results combined using a fixed-effects meta-analysis. In addition, the proportion of variation in CDR explained by circRNAs versus their cognate linear mRNAs was calculated, and the average proportion of variation explained in the two datasets presented. Two of 148 meta-analysis CDR-associated circRNAs did not have a cognate linear RNA and were excluded from these analyses.

The independence of circRNA-AD associations from AD-associated changes in estimated brain cell-type proportions was demonstrated using a similar regression-based approach. Library-size-normalized counts of the CDR-associated circRNAs and computationally deconvoluted estimated proportions of neurons, oligodendrocytes and microglia were included in the same regression models predicting CDR. The deconvoluted estimated astrocyte proportion was not included, to avoid multicollinearity and also because astrocyte- and neuron-estimated proportions are strongly inversely correlated. AD-associated circRNAs that retained a significant (P<0.05) association in these combined models are considered independent. These regression analyses were performed for all 148 CDR-associated circRNAs in both the discovery and the replication datasets, and the results combined using a fixed-effects meta-analysis.

Pre-symptomatic AD Bootstrapped Correlation Coefficient Analyses. In the discovery and replication datasets, a small number of individuals with PreSympAD—that is, neuropathological evidence of AD but, at most, very mild dementia (CDR≤0.5)—were included. It was investigated whether changes in expression in the PreSympAD brains were similar to the changes observed in SympAD—that is, neuropathological evidence of AD and dementia (CDR≥1) compared to controls.

First, a cortical circRNA differential expression analysis was performed between SympAD and controls and then between PreSympAD and controls, using the same methods as described above. Then, for all circRNAs that were not automatically filtered out by DESeq2 due to low expression, the correlation was calculated between the log2(fold change) (log2FC, effect size) observed in the PreSympAD analysis and that observed in the SympAD analysis. If the SympAD versus control brain differentially expressed circRNAs demonstrate similar changes in expression in the PreSympAD, it would be expected that the correlation between the log2FC values for these circRNAs would be stronger than those from the non-significant background circRNAs. This was tested for by performing 10,000 bootstrap simulations to identify a bias-corrected and -accelerated 95% confidence interval for the two log2FC correlation coefficients—one for the SympAD-associated circRNAs and the other for the non-significant background circRNAs. P values were generated for the significantly associated distribution being higher than the background distribution, using a one-tailed Kolmogorov-Smirnov test. This analysis was performed in the discovery dataset and all cortical regions in the replication dataset to assess for cortex regional differences in circRNA expression changes in PreSympAD. Bootstrap correlation coefficients and confidence intervals were generated using the boot R package v.1.3-20.

Receiver Operating Characteristic (ROC) curve and Area under the curve (AUC) analyses. To evaluate the predictive ability of AD-associated circRNAs, logistic regression models predicting AD case status were calculated in both the discovery and the replication MSBB datasets. Each dataset was subsetted to include only definite AD cases and controls, and three models were calculated. The first model (base) included the following as covariates: PMI, median TIN, AOD, batch, sex, genetic ancestry and number of APOE4 alleles. The second model (circ) included the top 10, most significantly CDR-associated, circRNAs from the meta-analysis. The third model (base+circ) combined the variables of the first two models. Receiver operating characteristic curves and areas under the curve were calculated using the R package pROC v.1.12.1.

Relative importance analyses. The number of APOE4 alleles—the most common genetic risk factor for AD—and the estimated proportion of neurons are known to contribute to the observed variation in AD quantitative traits such as CDR and Braak score. The relative importance of circRNA expression compared with these known contributors was assessed using the relaimpo R package, v.2.2.3. To do this, first the library-size-normalized counts of the top 10 most significant AD-trait associated circRNAs were adjusted for the same covariates used in the differential expression analyses: PMI, median TIN, AOD, batch, sex and genetic ancestry. Then these normalized, adjusted counts were included, first individually and then together in a multivariate model, with the number of APOE4 alleles and estimated neuronal proportion in the same linear regression models predicting CDR, Braak score, or mean number of plaques (only available in the replication MSBB dataset). The relative contribution of each of the model variables to the variation in the predicted AD quantitative trait was assessed using the lmg method of the relaimpo package. Thus, the contribution of each of the top 10, most meta-analysis significant circRNAs was compared with the number of APOE4 alleles and estimated neuronal proportions, both individually and when included together in the same model. These analyses were conducted in both the discovery dataset and all four cortical regions of the replication dataset, selecting the top 10 most meta-analysis significant circRNAs from each region-specific meta-analysis.

Network Co-expression Analyses. CircRNA and protein-coding linear transcript co-expression networks from AD and control samples were generated to infer the biological and pathological relevance of circRNAs based on the linear transcripts with which they co-expressed. First library-size-normalized, circRNA and linear transcript counts, from the same samples, were adjusted for the differential expression analysis covariates—PMI, median TIN, AOD, batch, sex and genetic ancestry. For the purpose of network generation, all circRNAs but only the top 10,000 most variable protein-coding linear transcripts were included, to reduce the computational burden. Gene co-expression networks were computed from these combined counts based on Spearman's correlation using multiscale, embedded-gene, co-expression network analysis (MEGENA, v.1.3.6). Briefly, this method leverages planar, maximally filtered, graph techniques to identify compact gene expression networks, and has been independently demonstrated to have high module conservation with, and to identify more modules than, the older weighted correlation network analysis (WGCNA) method. Importantly, this method identifies hierarchical networks with submodules existing within larger parent modules, when possible. As such the same linear transcript or circRNA may be assigned to multiple modules. After module identification, each module's eigengene was calculated using the WGCNA R package v.1.63. To identify significant associations between modules and CDR, two-tailed, P-value-generating regression analyses were performed between the module eigengenes and CDR, adjusting for the differential expression covariates. The significance of the module eigengene association with CDR was determined using a two-tailed, Student's t-test. Significant gene enrichment and pathway associations were identified for each module by extracting the linear transcript module members and processing them through the FUMA software's hypergeometric one-tailed test, with protein-coding genes as the background gene list. Finally, networks were visualized using the igraph R package v.1.2.1.

MicroRNA Binding Site Prediction. A fasta file of circRNA sequences was generated using the getcircfasta.py script provided with DCC software. The present study predicted miRNA-binding sites in these circRNA sequences using the targetscan_70.pl script provided with the TargetScan70 database, March 2018 release. When multiple isoforms of the same circRNA were predicted to have different numbers of binding sites for the same miRNA, the greatest number of predicted binding sites was selected to present at the gene level. Predicted targets of miRNA regulation were identified using the March 2018 release of the TargetScanHuman database.

Statistical Analysis. Differential expression of circRNAs was tested for using DESeq2 v.1.18.1 to perform negative binominal family logistic regressions with a two-tailed Wald test to determine significance. CircRNA association effect size correlations were tested for using Pearson's correlation with significance determined using a two-tailed, Student's t-test. The independence of circRNA-AD associations from AD-associated changes in cognate linear mRNAs or changes in estimated brain cell-type proportions, using linear regression analyses with significance determined using two-tailed, Student's t-tests. One-tailed P values were calculated for the significance of overlap between different sets of differentially expressed circRNAs using the SuperExactTest R package v.1.0.0. Whether bootstrapped effect-size correlation distributions between SympAD-associated circRNAs were greater than the background distribution was calculated using a one-tailed Kolmogorov-Smirnov test. The proportion of variation in quantitative AD traits explained by circRNAs and other contributors was calculated using linear regression followed by a relative importance analysis using the relaimpo R package v.2.2.3. CircRNA and linear mRNA co-expression network modules were generated based on Spearmann's correlation using MEGENA v.1.3.6. Module eigengenes were calculated and their association determined with CDR using linear regression, with the significance determined using two-tailed, Student's t-tests. Co-expression module enrichment for AD-related pathways was determined using a one-tailed hypergeometric test performed using FUMA software. For parametric tests, data distribution was assumed to be normal, but this was not formally tested. All statistical analysis was done using R statistical software.

Data Availability. Knight ADRC dataset: NG00083. Sequencing information derived from ADAD samples is protected and requires additional authorization from DIAN for access. Mount Sinai Brain Bank replication dataset: syn3159438.

### Results

(i) Study Design

The study design included calling and quantifying circRNA counts in two independent RNA-seq datasets derived from neuropathologically confirmed (Mirra S. S. et al., Neurology 41, 479-486 (1991); Khachaturian, Z. S., Arch. Neurol. 42, 1097-1105 (1985)) AD case and control brain tissues. In the discovery dataset, 150-nucleotide (nt), paired-end, ribosomal (r)RNA-depleted, RNA-seq data were generated from frozen parietal cortex tissue donated by 96 individuals (13 controls and 83 AD cases). These individuals were assessed at the Knight ADRC of Washington University School of Medicine, and their demographic, clinical severity and neuropathological information were recorded. For replication, an independent, publicly available, Accelerating Medicines Partnership-Alzheimer's Disease MSBB dataset (syn3157743) (Wang M. et al., Sci Data 5, 180185 (2018)) was leveraged. In brief, the MSBB dataset includes 100-nt, single-end, rRNA-depleted, RNA-seq data derived from 195 samples (40 controls, and 89 definite, 31 probable and 35 possible AD cases) of inferior frontal gyrus tissue (Brodmann area (BM) 44), as well as data derived from three additional cortical regions (frontal pole (BM10), superior temporal gyrus (BM22) and parahippocampal gyrus (BM36)). Demographic, clinical severity and neuropathological information for all individuals in the MSBB dataset, separated by cortical region.

STAR software (Dobin, A. et al., Bioinforma. Oxf. Engl. 29, 15-21 (2013), incorporated herein by reference) was used in chimeric read-detection mode to align the reads from both RNA-seq datasets to the GENCODE-annotated human reference genome (GRCh38). Chimeric reads were further processed and filtered using DCC software to identify backsplice junctions. Finally, the backsplice junction counts were collapsed on to their linear gene of origin to generate a set of high-confidence circRNA counts for downstream analyses (see Methods). Using this pipeline, 3,547 circRNAs in the discovery dataset (Knight ADRC) and an average of 3,924 circRNAs in the four regions of the replication dataset (MSBB) were called. Replication analyses were focused primarily on the BM44-derived data, because the largest overlap was observed between the circRNAs called in this region and the parietal dataset (FIG. 5). Analyses in the other MSBB cortical regions yielded similar results.

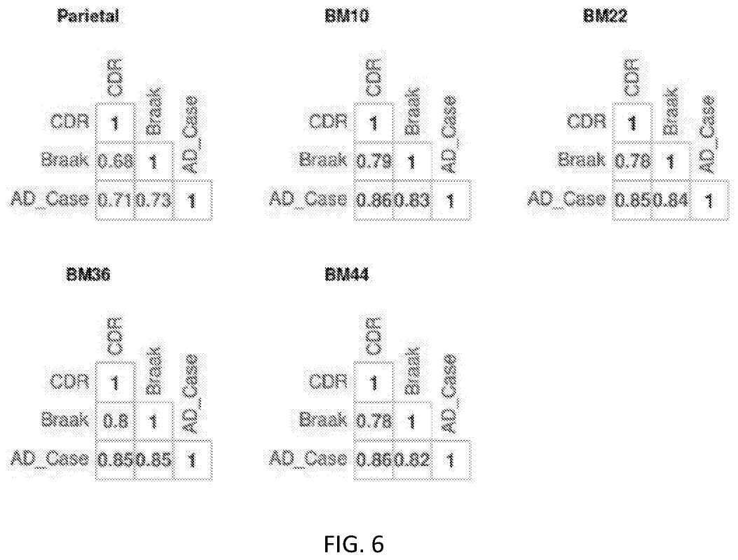

DESeq2 software (Love, M. I. et al., Genome Biol. 15, 550 (2014)) was used to perform circRNA differential expression analyses for neuropathological AD case-control status as well as for correlation with AD quantitative traits: Braak score and clinical dementia rating at expiration/death (CDR). Braak score is a neuropathological measure of AD severity determined by the number and distribution of neurofibrillary tau tangles throughout the brain. It ranges from 0 (absent, at most incidental tau tangles) to 6 (severe, extensive tau tangles in neocortical areas). CDR is a clinical measure of cognitive impairment with a range from 0 (no dementia) to 3 (severe dementia). These quantitative measures capture different aspects of the pathological mechanisms underlying AD and consequently are not perfectly correlated with each other or with AD case status (FIG. 6). Thus, each trait was analyzed separately, modeling the ordinal measures as continuous variables. All analyses were adjusted for postmortem interval (PMI), RNA quality as measured by median transcript integrity number (TIN), age at death (AOD), batch, sex and genetic ancestry as represented by the first two principal components derived from genetic data (see Methods). The circRNA analyses were extended to pre-symptomatic and autosomal dominant AD to investigate whether circRNA expression changes occurred before substantial symptom onset and whether these changes were restricted to sporadic AD. Finally, the AD relevance and potential disease-influencing mechanisms of AD-associated circRNAs were investigated through relative importance, network co-expression and miRNA-binding site-prediction analyses.

(ii) Discovery Analysis to Identify AD Differentially Expressed circRNAs

In the circular-transcriptome-wide discovery analysis (n_{CDR}=96, n_{Braak}=86, n_{control}=13, n_{AD}=83), 31 circRNAs were significantly correlated with CDR, passing a false discovery rate (FDR) of 0.05. The most significantly correlated circRNA was circHOMER1 (log2(fold change) −0.28 per unit of CDR, P=8.22×10−12). circCDR1-AS (log2(fold change) 0.17 per unit of CDR, P=3.18×10−2) was only nominally correlated with CDR, but, in contrast to the previous report (K+Lukiw, W. J. et al., Front Genet. 4, (2013)), its expression was observed to be upregulated with increasing dementia severity.

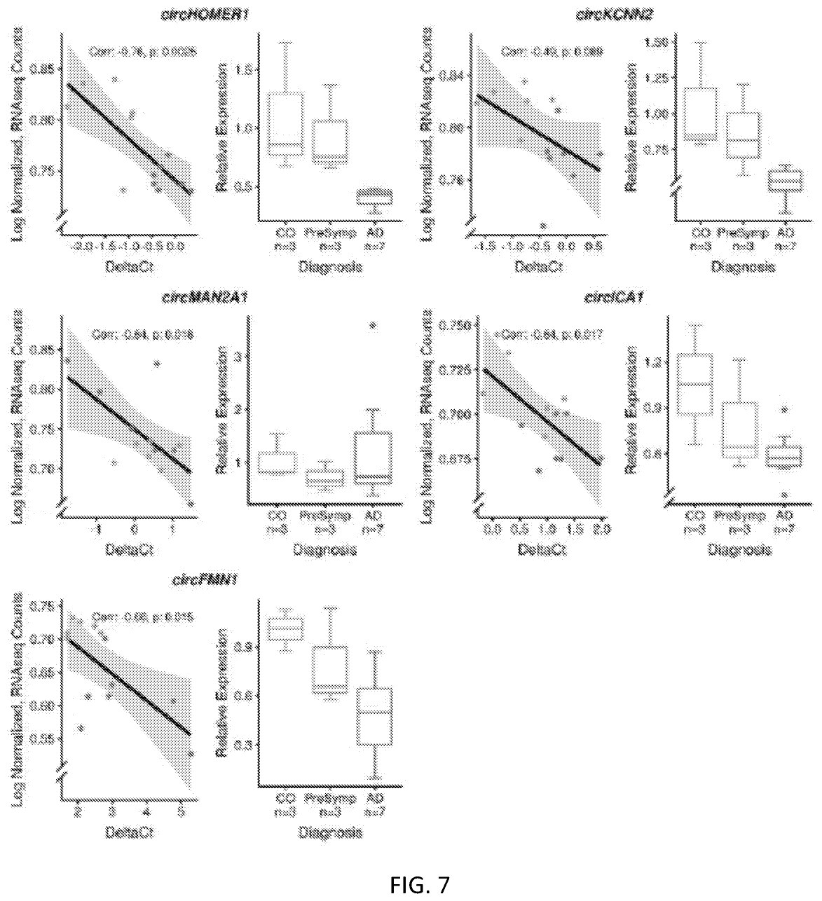

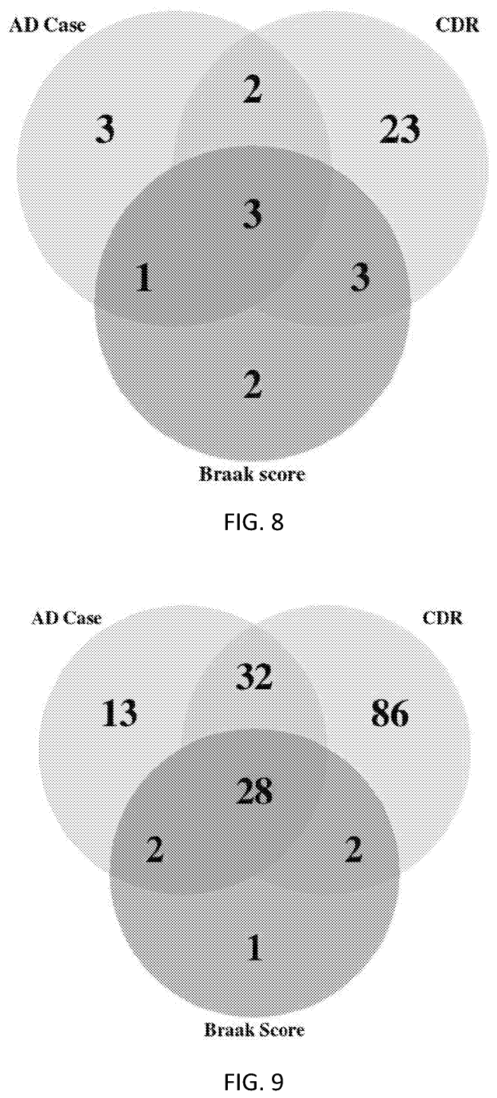

Also identified were circRNAs significantly associated with the two other complementary AD traits: Braak score (nine circRNAs passed FDR) and neuropathological AD versus control status (nine circRNAs passed FDR). These analyses yielded both AD-trait-specific associations and circRNAs that were consistently associated across all AD traits investigated. Three circRNAs passed FDR correction for all three traits. For example, in addition to the CDR association, circHOMER1 was also significantly associated with Braak score (P=1.19×10⁻⁷) and AD versus control status (P=2.76×10⁻⁶). In general, circRNAs associated with one AD trait were also, at least nominally, associated (P<0.05) with the remaining two traits. The RNA-seq findings were validated for 5 circRNAs using an orthogonal quantitative PCR approach with 13 discovery dataset RNA samples (n_{control}=3, n_{PreSympAD}=3, n_{AD}=7). A strong correlation was demonstrated between RNA-seq-derived counts for the five circRNA transcripts and the GAPDH-normalized ΔCt values (median absolute correlation: 0.64; FIG. 7). Importantly, consistent directions of effect were also observed, thereby validating the RNA-seq results (FIG. 7). Altogether, 37 circRNAs were identified in the discovery parietal cortex dataset analysis that were significantly associated with at least one AD trait (FIG. 8).

(iii) Replication and Meta-Analysis of circRNA Differential Expression Using an Independent AD Dataset

Replication analyses were performed in the MSBB BM44 dataset (n_{CDR}=195, n_{Braak}=188, n_{control}=40, n_{Definite AD}=89). Twenty-seven of the 31 CDR-associated circRNAs identified in the discovery analysis, were similarly correlated with CDR in the replication analysis, with, at minimum, a nominal P value, the same directions of effect and comparable effect sizes (effect size Pearson's correlation: 0.97, P=1.69×10⁻¹⁷). For example, decreasing circHOMER1 expression with increasing dementia severity (log2(fold change) −0.13 per unit of CDR, P=2.27×10⁻⁹) was replicated. A meta-analysis of the discovery and replication results revealed a total of 148 circRNAs that were significantly correlated with CDR after FDR correction, with 33 passing the stringent gene-based, Bonferroni multiple test correction of 5×10⁻⁶ (Table 1), including circHOMER1 (P=2.21×10⁻¹⁸) and circCDR1-AS (P=2.83×10⁻⁸), among others.

TABLE 1
Cortical circRNAs are significantly associated with AD case status, dementia severity and neuropathological severity.
CircRNA association with AD traits in the discovery Knight ADRC parietal dataset, replication MSBB BM44 dataset
and meta-analyses. Presented are the log2(fold changes) and P values generated via a Wald-log test for the discovery
(nCDR = 96) and replication (nCDR = 195) analyses, as well as the inverse/Stouffer's method combined
P values for the meta-analyses. Discovery and replication analyses were adjusted for PMI, RNA quality (median
TIN), age at death, batch, sex and genetic ancestry (principal components 1-2). Chr, chromosome.

| | | CDR-discovery | | CDR-replication | | Meta-analysis | | |
|---|---|---|---|---|---|---|---|---|
| | | log₂(fold | | log₂(fold | | CDR | Braak score | AD case |
| CircRNA | Chr | change) | P | change) | P | P | P | P |
| circHOMER1 | 5 | −0.28 | 8.22 × 10⁻¹² | −0.13 | 2.27 × 10⁻⁹ | 2.21 × 10⁻¹⁸ | 4.77 × 10⁻¹² | 4.35 × 10⁻¹⁰ |
| circDOCK1 | 10 | 0.30 | 8.49 × 10⁻⁶ | 0.20 | 7.55 × 10⁻⁸ | 6.47 × 10⁻¹² | 8.68 × 10⁻⁷ | 3.74 × 10⁻⁶ |
| circKCNN2 | 5 | −0.12 | 7.27 × 10⁻⁴ | −0.12 | 1.93 × 10⁻⁹ | 1.47 × 10⁻¹¹ | 4.43 × 10⁻⁸ | 8.38 × 10⁻⁸ |
| circMAN2A1 | 5 | 0.23 | 2.46 × 10⁻⁴ | 0.17 | 2.92 × 10⁻⁷ | 5.59 × 10⁻¹⁰ | 1.25 × 10⁻⁶ | 3.75 × 10⁻⁹ |
| circST18 | 8 | 0.37 | 1.27 × 10⁻⁴ | 0.28 | 6.60 × 10⁻⁷ | 6.80 × 10⁻¹⁰ | 7.30 × 10⁻⁶ | 1.22 × 10⁻⁹ |
| circATRNL1 | 10 | −0.13 | 2.42 × 10⁻³ | −0.13 | 4.15 × 10⁻⁸ | 9.47 × 10⁻¹⁰ | 4.26 × 10⁻⁵ | 2.73 × 10⁻⁶ |
| circEXOSC1 | 10 | 0.14 | 3.66 × 10⁻² | 0.18 | 8.13 × 10⁻⁹ | 7.92 × 10⁻⁹ | 6.22 × 10⁻⁵ | 1.27 × 10⁻⁶ |
| circICA1 | 7 | −0.16 | 7.40 × 10⁻⁵ | −0.11 | 2.33 × 10⁻⁵ | 1.77 × 10⁻⁸ | 3.43 × 10⁻² | 2.08 × 10⁻⁶ |
| circFMN1 | 15 | −0.16 | 1.01 × 10⁻⁴ | −0.11 | 2.13 × 10⁻⁵ | 2.07 × 10⁻⁸ | 2.12 × 10⁻⁶ | 3.79 × 10⁻⁶ |
| circRTN4 | 2 | 0.14 | 8.36 × 10⁻³ | 0.13 | 2.72 × 10⁻⁷ | 2.18 × 10⁻⁸ | 6.96 × 10⁻⁸ | 4.81 × 10⁻⁹ |
| circCDR1-AS | 23 | 0.17 | 3.18 × 10⁻² | 0.19 | 4.90 × 10⁻⁸ | 2.83 × 10⁻⁸ | 1.54 × 10⁻³ | 5.29 × 10⁻¹² |
| circMAP7 | 6 | 0.17 | 1.83 × 10⁻⁵ | 0.10 | 1.66 × 10⁻⁴ | 5.51 × 10⁻⁸ | 1.07 × 10⁻⁶ | 5.41 × 10⁻⁸ |
| circTTLL7 | 1 | 0.18 | 2.59 × 10⁻³ | 0.16 | 3.42 × 10⁻⁶ | 6.18 × 10⁻⁸ | 1.22 × 10⁻⁶ | 1.07 × 10⁻⁷ |
| circFANCL | 2 | 0.21 | 9.12 × 10⁻³ | 0.15 | 9.88 × 10⁻⁷ | 7.65 × 10⁻⁸ | 1.75 × 10⁻³ | 1.11 × 10⁻³ |
| circEPB41L5 | 2 | −0.13 | 1.12 × 10⁻³ | −0.09 | 1.02 × 10⁻⁵ | 7.84 × 10⁻⁸ | 1.71 × 10⁻⁵ | 2.67 × 10⁻⁴ |
| circCORO1C | 12 | 0.12 | 7.19 × 10⁻⁴ | 0.11 | 2.20 × 10⁻⁵ | 1.14 × 10⁻⁷ | 7.97 × 10⁻⁶ | 2.45 × 10⁻⁷ |
| circDGKI | 7 | −0.12 | 3.86 × 10⁻² | −0.14 | 2.42 × 10⁻⁷ | 1.41 × 10⁻⁷ | 3.78 × 10⁻³ | 1.05 × 10⁻³ |
| circKATNAL2 | 18 | −0.14 | 2.39 × 10⁻² | −0.21 | 5.78 × 10⁻⁷ | 1.55 × 10⁻⁷ | 2.11 × 10⁻³ | 8.74 × 10⁻⁵ |
| circWDR78 | 1 | 0.14 | 5.84 × 10⁻⁴ | 0.11 | 3.59 × 10⁻⁵ | 1.57 × 10⁻⁷ | 2.62 × 10⁻⁴ | 2.95 × 10⁻⁵ |
| circADGRB3 | 6 | −0.07 | 1.10 × 10⁻² | −0.07 | 2.20 × 10⁻⁶ | 1.94 × 10⁻⁷ | 5.97 × 10⁻³ | 1.47 × 10⁻³ |
| circPLEKHM3 | 2 | −0.19 | 6.13 × 10⁻⁶ | −0.10 | 1.00 × 10⁻³ | 2.32 × 10⁻⁷ | 3.77 × 10⁻⁴ | 4.13 × 10⁻⁶ |
| circERBIN | 5 | 0.25 | 1.34 × 10⁻³ | 0.17 | 2.92 × 10⁻⁵ | 2.67 × 10⁻⁷ | 2.42 × 10⁻⁴ | 1.20 × 10⁻⁵ |
| circPICALM | 11 | 0.07 | 1.29 × 10⁻² | 0.08 | 4.63 × 10⁻⁶ | 4.54 × 10⁻⁷ | 3.12 × 10⁻⁶ | 3.35 × 10⁻⁸ |
| circRNASEH2B | 13 | 0.20 | 3.57 × 10⁻³ | 0.14 | 3.13 × 10⁻⁵ | 7.11 × 10⁻⁷ | 1.72 × 10⁻³ | 4.63 × 10⁻³ |
| circPDE4B | 1 | −0.13 | 5.84 × 10⁻³ | −0.11 | 1.98 × 10⁻⁵ | 7.47 × 10⁻⁷ | 1.94 × 10⁻³ | 5.33 × 10⁻⁵ |
| circPHC3 | 3 | 0.16 | 7.43 × 10⁻⁴ | 0.11 | 1.40 × 10⁻⁴ | 7.99 × 10⁻⁷ | 2.09 × 10⁻² | 1.01 × 10⁻² |
| circFAT3 | 11 | −0.23 | 4.75 × 10⁻³ | −0.21 | 3.11 × 10⁻⁵ | 9.31 × 10⁻⁷ | 8.21 × 10⁻³ | 2.04 × 10⁻⁴ |
| circMLIP | 6 | −0.08 | 5.75 × 10⁻² | −0.10 | 3.41 × 10⁻⁶ | 2.24 × 10⁻⁶ | 7.22 × 10⁻⁶ | 2.71 × 10⁻⁷ |
| circLPAR1 | 9 | 0.17 | 2.17 × 10⁻² | 0.20 | 1.72 × 10⁻⁵ | 2.68 × 10⁻⁶ | 1.49 × 10⁻³ | 4.58 × 10⁻⁶ |
| circSLAIN2 | 4 | 0.14 | 5.25 × 10⁻⁴ | 0.12 | 5.62 × 10⁻⁴ | 2.70 × 10⁻⁶ | 2.51 × 10⁻² | 2.63 × 10⁻⁵ |
| circSPHKAP | 2 | −0.39 | 1.48 × 10⁻³ | −0.27 | 3.16 × 10⁻⁴ | 3.32 × 10⁻⁶ | 2.88 × 10⁻² | 2.44 × 10⁻¹ |
| circYY1AP1 | 1 | 0.20 | 4.47 × 10⁻⁴ | 0.11 | 9.71 × 10⁻⁴ | 4.40 × 10⁻⁶ | 1.83 × 10⁻⁴ | 1.15 × 10⁻³ |
| circDNAJC6 | 1 | 0.16 | 6.63 × 10⁻³ | 0.11 | 1.27 × 10⁻⁴ | 4.99 × 10⁻⁶ | 2.04 × 10⁻⁵ | 8.21 × 10⁻⁶ |

Similarly, five of the nine circRNAs that were correlated with Braak score in the discovery dataset replicated in the MSBB dataset (effect size Pearson's correlation=0.99; P=9.29×10⁻⁶). A total of 33 circRNAs were significantly associated with Braak score after FDR correction in the meta-analysis. Finally, five of nine circRNAs associated with AD case-control status replicated in the MSBB dataset (effect size Pearson's correlation=0.99; P=6.12×10⁻⁵) and 75 circRNAs were associated with AD case-control status after FDR correction in the meta-analysis.

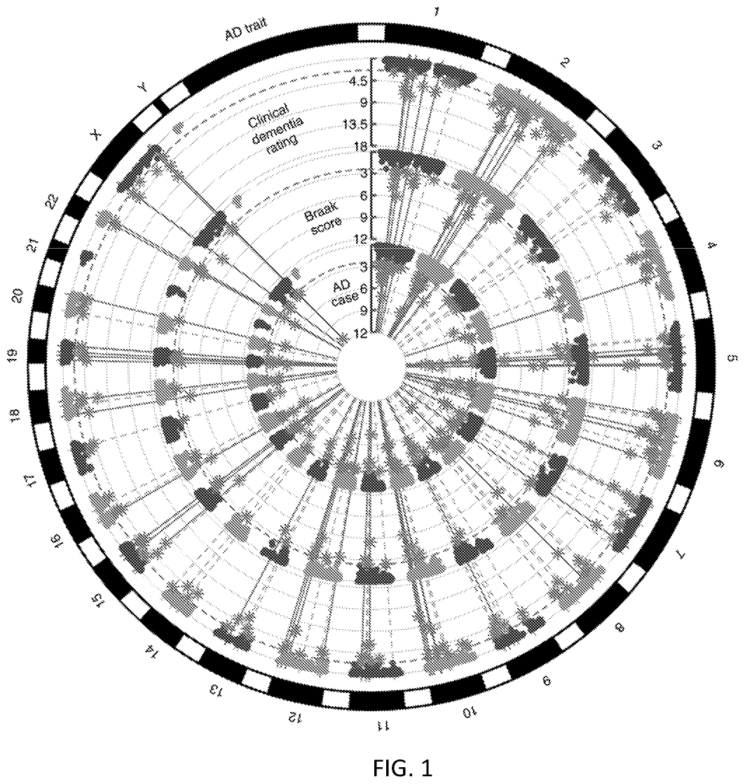

Overall, 164 circRNAs were identified that were significantly associated with at least one AD trait in the meta-analyses (FIG. 1). Twenty-eight of these circRNAs, including circHOMER1 and circCORO1C, were significantly associated with all three traits investigated (FIG. 9). Nine cross-trait circRNA associations had P values passing the gene-based stringent threshold of 5×10⁻⁶ (Table 1). Altogether, these results support a consistent, replicable and highly significant association between changes in circRNA expression and AD traits.

(iv) AD-Associated Changes in circRNA Expression Demonstrate Independence from AD-Associated Changes in Their Cognate Linear mRNAs and Estimated Brain Cell-Type Proportions

CirRNAs and their cognate linear mRNAs can demonstrate independent expression (You, X. et al., Nat. Neurosci. 18, 603-610 (2015)), but some level of correlation is expected given the shared genomic origin and biogenesis machinery. This correlation is also technically biased because most RNA-seq reads covering a circRNA transcript will not contain the circRNA-defining backsplice junction, and thus will be incorrectly counted as originating from a linear mRNA rather than a circRNA transcript (Rybak-Wolf, A. et al., Mol. Cell 58, 870-885 (2015)). For example, linear forms of circCDR1-AS are expressed at such low levels that they have been historically undetectable (Piwecka, M. Et al., Science 357, eaam8526; Barrett, S. P. et al., PLoS Genet. 13 e1007114 (2017)). However, abundant ‘linear’ CDR1-AS counts were observed in the present linear mRNA quantification, consistent with the known technical bias. This artifact is expected to bias circRNA-AD associations to the null, when the relatively less abundant circRNAs are included together in the same regression models as their cognate linear mRNAs. Nevertheless, it was demonstrated that most of the CDR-associated changes in circRNA expression are independent from CDR-associated changes in their cognate linear mRNAs using a regression-based approach.

In the meta-analysis of the discovery and replication, linear and circRNA, combined regression results, it was observed that 109 of 146 circRNAs retain a significant association (P<0.05) with CDR—for example, circHOMER1 (P=3.11×10⁻⁶) or circDOCK1 (P=1.65×10⁻⁶)—demonstrating an independent association. In addition, 62 CDR-associated circRNAs had association P values less than the association P values of their cognate linear mRNAs, and 78 CDR-associated circRNAs explained as much or more of the variation in CDR compared to their cognate linear mRNAs. In a separate analysis, a similar regression-based approach was employed to demonstrate that most (106 of 148) CDR-associated circRNAs—for example, circHOMER1 (P=8.15×10⁻¹³) or circDOCK1 (P=1.03×10⁻⁵)—are similarly independent of AD-associated changes in estimated neuronal and other brain cell-type proportions Li, Z. et al., Genome Med. 10, 43 (2018)). Together, these results demonstrate that the majority of AD-circRNA associations are independent of AD-associated changes in linear mRNA or estimated brain cell-type proportions.

(v) AD-Associated Changes in circRNA Expression are Consistent Across Cortical Regions

The MSBB dataset also includes RNA-seq data derived from three additional brain cortical regions: BM10, BM22, and BM36. To determine whether AD-associated changes in circRNA expression were consistent across the cortex, circular-transcriptome-wide analyses were performed in these additional datasets. As before, circRNA correlation with CDR and Braak score, as well as circRNA association with AD case-control status was investigated. Three sets of meta-analyses were performed with the parietal discovery results, one for each of the additional cortical regions: BM10, BM22, and BM36. Then a comparison was made of these results with the BM44 meta-analysis results to identify consistent AD-associated circRNA expression changes across these cortical regions.

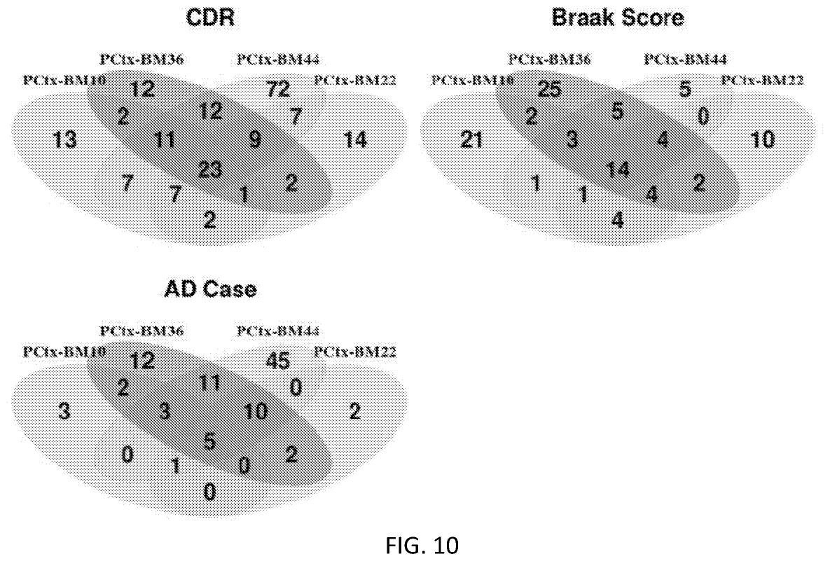

The present Example identified 23 circRNAs that were significantly associated with CDR in all four meta-analyses, with comparable effect sizes and the same directions of effect (overlap P=1.60×10⁻⁹⁴; FIG. 10). Similarly, 14 circRNAs were identified that were significantly associated with Braak score (overlap P=1.38×10⁻⁷⁰; FIGS. 10) and 5 that were significantly associated with AD case status (overlap P=3.90×10⁻²⁶; FIG. 10) with consistent directions of effect in all four meta-analyses. Three circRNAs, circHOMER1, circKCNN2 and circMAN2A1, were significantly associated with all three AD traits in all four meta-analyses. Eleven circRNAs were associated with the two quantitative AD traits in all four meta-analyses: circDGKB, circDNAJC6, circDOCK1, circERBIN, circFMN1, circHOMER1, circKCNN2, circMAN2A1, circMAP7, circSLAIN1 and circST18.

The MSBB dataset includes an additional measure of neuropathological severity, mean number of amyloid plaques. Results for circRNA correlation with mean number of plaques were consistent with the other AD traits in all MSBB cortical regions (Supplementary Results). Together, these results suggest that expression changes in some circRNAs are a consistent phenomenon across cortical regions in the context of AD.

(vi) Evidence Supporting circRNA Differential Expression in Pre-Symptomatic AD

The present Example investigated for early AD-related changes in circRNA expression in a small number (n_{Discovery}=6 and n_{Replication}=18) of individuals with pre-symptomatic AD—that is, neuropathological evidence of AD but, at most, very mild dementia (CDR≤0.5).

First circRNA expression was compared between pre-symptomatic AD (PreSympAD) versus controls (control n_{Discovery}=13, control n_{Replication}=40) in each dataset individually, but no significant circRNA differential expression was detected. Nevertheless, several nominal associations were identified, with directions and magnitudes of effect (log2(fold change)) consistent with those observed in complementary analyses identifying circRNA differential expression between symptomatic (CDR≥1) individuals with AD neuropathology (SympAD) versus controls in the BM44 dataset (n_{SympAD}=137), but not in the smaller parietal dataset (n_{SympAD}=77).

These results suggested that changes in circRNA expression occur in PreSympAD, but there were too few individuals to detect this on a transcriptome-wide basis. If this hypothesis is correct, then the effect size correlation between nominally PreSympAD-associated circRNAs and significantly SympAD-associated circRNAs should be stronger for the latter compared with the background, non-SympAD-associated circRNAs. Thus, bootstrapped confidence intervals were generated for the Pearson's correlation between effect sizes.

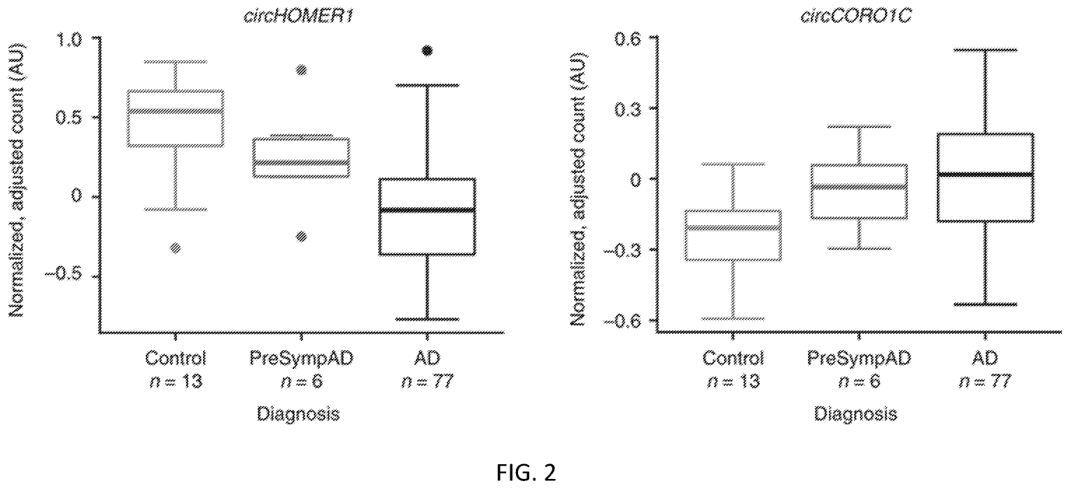

It was observed that the bootstrapped effect size correlation coefficient distribution for the SympAD-associated circRNAs was significantly higher than the background distribution in both the parietal discovery (14 SympAD-associated circRNAs, effect size correlation=0.67 (0.43, 0.90), versus 713 background circRNAs, effect size correlation=0.21 (0.14, 0.29), P<2.2×10⁻¹⁶; FIG. 2) and the BM44 replication (100 SympAD-associated circRNAs, effect size correlation=0.78 (0.68, 0.85) versus 1,544 background circRNAs, effect size correlation=0.36 (0.31, 0.41), P<2.2×10-16) datasets.

When these analyses were extended to the three other cortical regions of the MSBB dataset, it was also observed that there was similar evidence for pre-symptomatic changes in circRNA expression (P<2.2×10⁻¹⁶). The distribution width of the SympAD-associated, circRNA effect size correlations varied by cortical region: BM44˜BM36<BM22<parietal cortex<BM10, in a sequence reminiscent of the observed spatiotemporal progression of AD pathology within the cortex. Together, these results support early changes in circRNA expression in multiple cortical regions prior to substantial AD symptoms.

(vii) Changes in circRNA Expression are More Severe in Individuals with Autosomal Dominant AD

Autosomal dominant AD (ADAD) is an early onset form of AD caused by pathogenic mutations in APP, PSEN1 or PSEN2. It was investigated whether changes in circRNA expression also occur in the context of ADAD using parietal cortex-derived, RNA-seq data from 21 brains donated by individuals with ADAD who were enrolled in the Dominantly Inherited Alzheimer Network (DIAN) study. ADAD participant demographic, clinical and neuropathological data are presented in were recorded. The ADAD RNA-seq data were generated at the same time as the discovery RNA-seq data and circRNAs were called and filtered in both datasets simultaneously.

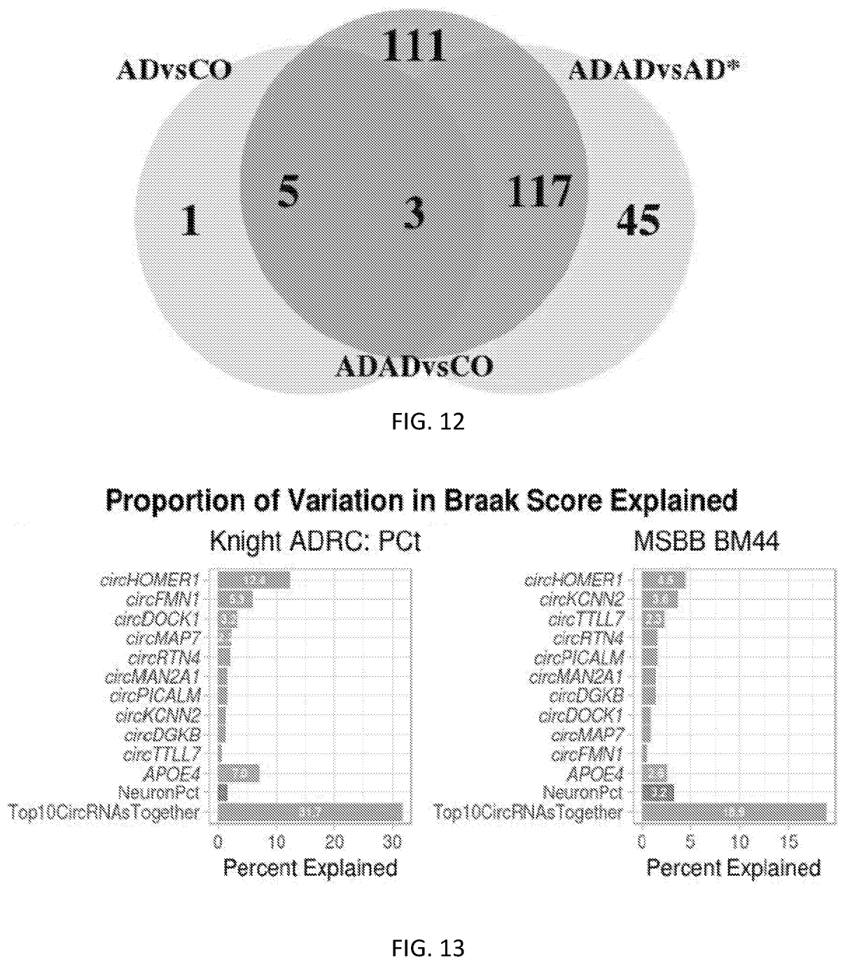

In a circular-transcriptome-wide analysis of circRNA differential expression between ADAD (n=21) and discovery dataset controls (n=13), 236 ADAD-associated circRNAs were identified that were significantly associated under the FDR threshold. These included almost all (8/9) AD case-control, status-associated circRNAs identified in the discovery analysis, with consistent directions of effect (FIG. 12). However, the magnitudes of effect were greater in the ADAD versus control analysis (for example, circHOMER1: AD versus control, log2(fold change) −0.64; ADAD versus control log2(fold change) −0.95).

To investigate whether the larger effect size was due to the greater pathological severity in the ADAD brains, a Braak score-adjusted, circRNA differential expression analysis was performed between ADAD and discovery dataset AD (samples with available Braak score: nADAD=17, nAD=73). This analysis identified 77 significantly differentially expressed circRNAs and 59 of these were identified in the ADAD versus control analysis (FIG. 12). As before, these 59 differentially expressed circRNAs had consistent directions of effect, and most (56/59) had greater magnitudes of effect when comparing controls versus AD versus ADAD. Altogether, these results demonstrate that changes in circRNA expression also occur in the context of ADAD and are more severe in magnitude, even when adjusting for neuropathological severity.

(viii) AD-Associated circRNAs Explain More of the Variation in AD Quantitative Measures than Number of APOE4 Alleles or Estimated Neuronal Proportion

Relative importance analyses were performed to assess the contribution of circRNA expression to the variation in AD quantitative traits: CDR and Braak score compared with two known contributors—number of APOE4 alleles (APOE4)—the most common genetic risk factor for AD—and the estimated proportion of neurons (EstNeuron).

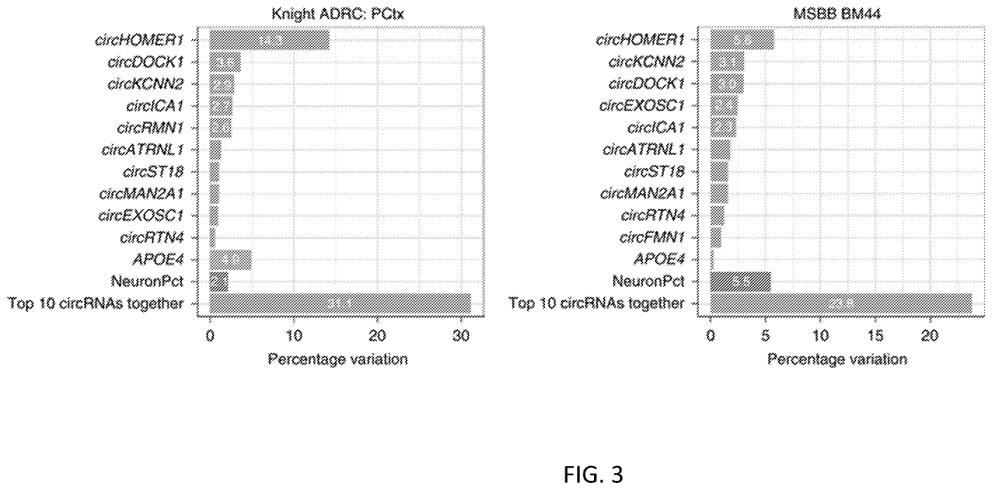

The top 10 meta-analysis significant CDR-associated circRNAs were selected for the proportion of variation-explained analyses. In the discovery dataset (ncoR=96), these circRNAs—included in the same multivariate model as APOE4 and EstNeuron—explained a total of 31.1% of the observed variation in CDR (FIG. 3). The BM44 replication dataset (n_{CDR}=195) results for the same circRNAs were consistent, with the circRNAs explaining a total of 23.8% of the variation in CDR (FIG. 3). In both the discovery and the replication datasets, some circRNAs individually, and the top 10 circRNAs together, were observed to explain more of the variation in CDR compared with APOE4 and EstNeuron (FIG. 3). The same pattern was observed when assessing the relative contribution of circRNAs to the observed variation in Braak score (FIG. 13) and also when analyzing the other MSBB tissues for contribution of circRNAs to variation in CDR , Braak score, and mean number of plaques (Supplementary Results). Finally, it was also observed that circRNAs explain more of the variation in Braak score in individuals with ADAD than APOE4 and EstNeuron (Supplementary Results).

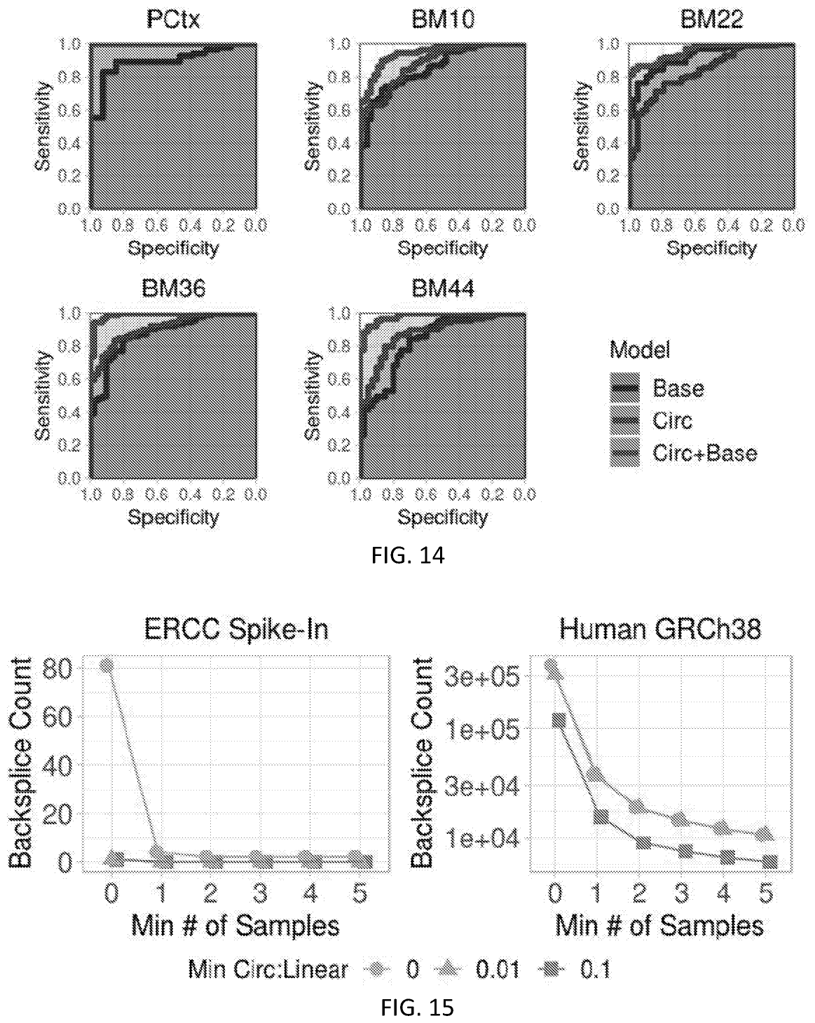

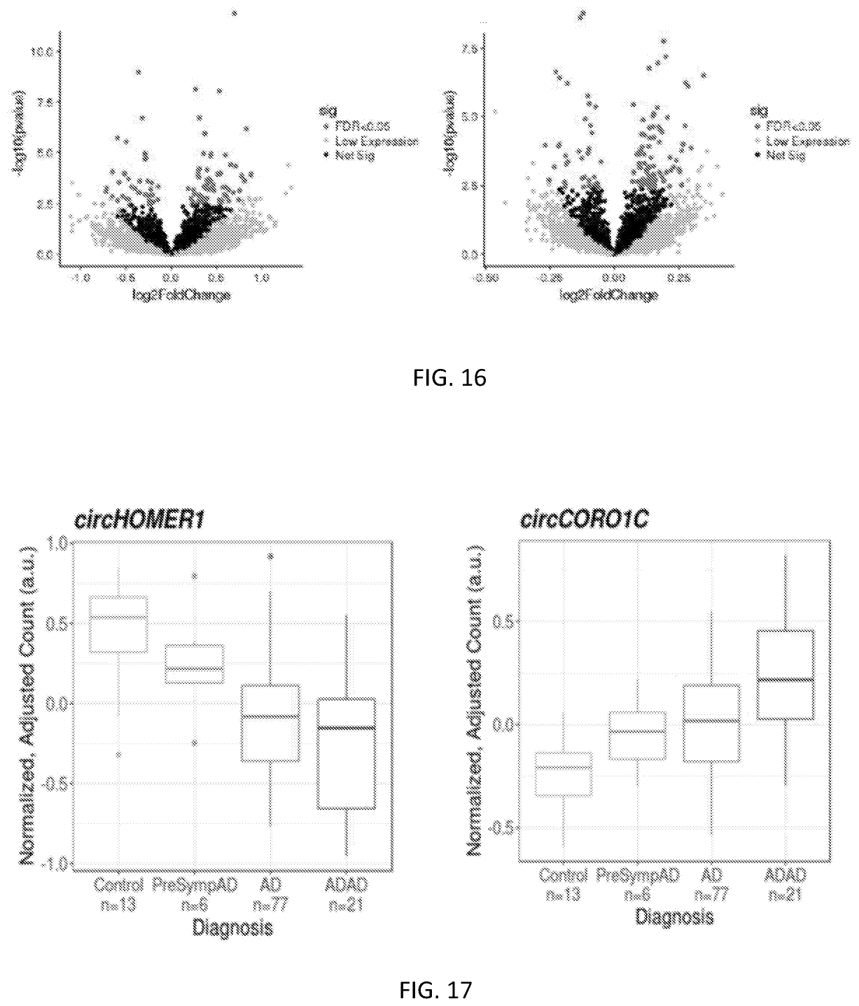

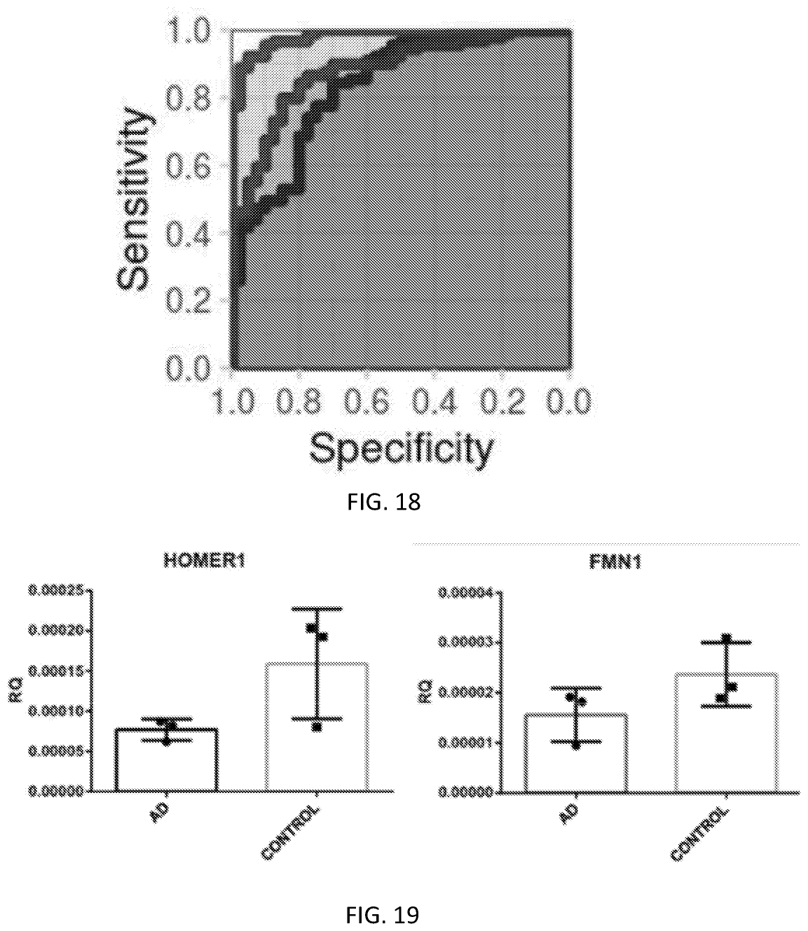

In addition to the proportion of variation analyses, the AD-predictive ability of the same 10 most meta-analysis significant CDR-associated circRNAs were also compared with the AD-predictive ability of baseline models that included number of APOE4 alleles and the differential expression covariates. Consistent with the relative importance analyses, it was found that circRNAs alone provided similar or greater predictive value compared with the baseline genetic-demographic models, and even improved the predictive ability when combined with the baseline genetic-demographic data (FIG. 14). Altogether, these results demonstrate that circRNA expression is strongly associated with AD quantitative traits and contributes notably to the variation in these AD severity measures.

(ix) Differentially Expressed circRNAs Co-Express with AD-Relevant Genes and Pathways

The analysis of circRNA co-expression with linear transcripts provides an opportunity to infer the biological and pathological relevance of circRNAs. Co-expression networks were computed in the discovery parietal dataset, as well as in each of the cortical regions of the MSBB dataset: BM10, BM22, BM36, and BM44, based on Spearman's correlation using MEGENA software. Further calculations were made of the correlation between the eigengenes of these networks and CDR.

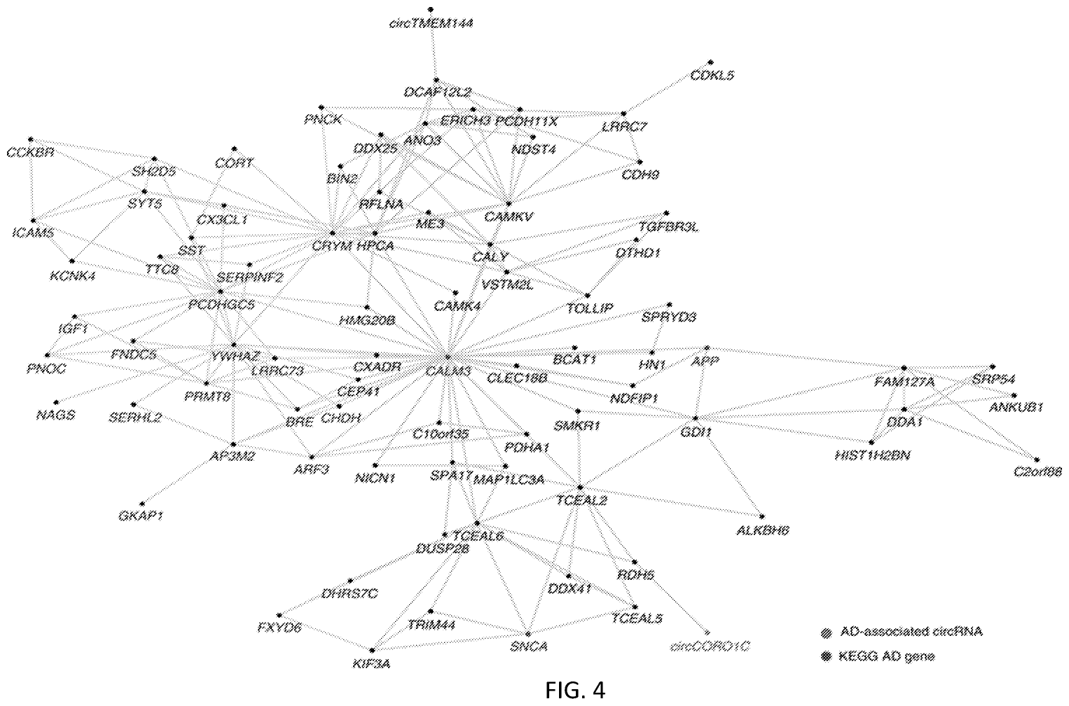

In the parietal dataset, 49 hierarchical co-expression modules were identified that were significantly correlated with CDR and contained at least one AD-associated circRNA. Similarly, in the MSBB BM44 dataset, 20 hierarchical co-expression modules were identified that significantly correlated with CDR and contained at least one AD-associated circRNA. CircHOMER1 was expressed in module c1_16 (module correlation with CDR, P=5.94×10⁻⁴) in the parietal dataset. This module included linear transcripts that are significantly enriched for AD pathways (KEGG (Kyoto Encyclopedia of Genes and Genomes) Alzheimer's Disease, 66/156 genes, adjusted P=1.07×10⁻¹⁵) and oxidative phosphorylation-related genes (KEGG Oxidative Phosphorylation, 58/115 genes, adjusted P=2.76×10⁻¹⁸). Similarly, the AD-associated circRNA circCORO1C co-expressed with the AD genes APP and SNCA in the BM44 dataset module c1_46 (module correlation with CDR, P=1.52×10⁻⁷; FIG. 4).

The MEGENA results in the other cortical regions of the MSBB dataset were consistent with AD-associated circRNAs co-expressing with AD-related genes and pathways. For example, APP was observed co-expressing with several AD-associated circRNAs in the BM22 module c1_14 (module correlation with CDR, P=2.39×10⁻⁶). Altogether, these results suggest an important role for circRNAs in AD.

(x) AD-Associated circRNAs Contain Binding Sites for miRNAs that Potentially Regulate AD-Associated Pathways and Genes

The functional consequences of circRNA expression is an area of active research. Although recent studies have demonstrated that circRNAs can regulate transcription (Li , X. et al., Mol. Cell 71, 428-442 (2018)) and even be translated (Legnini, I. et al., Mol. Cell 66, 22-37.e9 (2017)) their most well-characterized function is in miRNA regulation via sequestration (Legnini, I. et al., Mol. Cell 66, 22-37.e9 (2017); Hansen, T. B. et al., Nature 495, 384-388 (2013) incorporated herein by reference). For example, circCDR1-AS contains over 70 binding sites for miR-7 and reducing circCDR1-AS expression results in the downregulation of miR-7 target mRNAs. However, even a single miRNA-binding site on a circRNA appears sufficient to regulate miRNA function (Bai, Y. et al., J. Neurosci. Off. J. Soc. Neurosci. 38, 32-50 (2018)).

To identify miRNAs potentially regulated by AD-associated circRNAs, TargetScan70 software was used to predict miRNA-binding sites in circRNA sequences. The previously reported finding of over 70 miR-7-predicted binding sites was replicated in the circCDR1-AS sequence and binding sites for several intriguing miRNAs in the other AD-associated circRNAs were also predicted. CircATRNL1 contained 18 predicted binding sites for miR-136, increased expression of which triggers apoptosis in glioma cells. CircHOMER1 contained five predicted binding sites for miR-651, which is an miRNA predicted to target the AD-related genes PSEN1 and PSEN2 (Agarwal, V. et al., eLife 4, e05005 (2015) incorporated herein by reference). Finally, circCORO1C, which was identified as co-expressing with the AD-related genes APP and SNCA (FIG. 4), contains two predicted binding sites for miR-105, which is an miRNA predicted to target APP and SNCA. These bioinformatic results suggest that some AD-associated circRNAs may exert functional effects through miRNA regulation.

(xi) Supplementary Results

AD-associated changes in circRNA expression demonstrate independence from AD-associated changes in estimated cell-type proportion. AD pathology results in dramatic changes in brain cell-type proportions which can be estimated in RNA-seq data using computational deconvolution approaches. To demonstrate that AD-associated changes in circRNA expression are independent from AD-associated changes in brain cell-type proportions, regression analyses was performed with models including the differential expression covariates, library size-normalized circRNA counts, and computationally deconvoluted estimates of brain cell-type proportions. These regression analyses were performed in the discovery parietal and the replication BM44 datasets for all 148 meta-analysis significant CDR-association circRNAs. The fixed effects meta-analysis of these results are presented in Dube, U., Del-Aguila, J. L., Li, Z. et al. An atlas of cortical circular RNA expression in Alzheimer disease brains demonstrates clinical and pathological associations. Nat Neurosci 22, 1903-1912 (2019) Supplementary Table 17. We identify 106 CDR-associated circRNAs that retain a significant association (p-value<0.05, Supplementary Table 17) when included in the same model as brain cell-type proportion estimates, for example circHOMER1 (meta p-value: 8.15×10−13) and circDOCK1 (meta p-value: 1.03×10-05). These results demonstrate that the association between the majority of AD-associated circRNAs and CDR is independent from the CDR-associated changes in estimated cell-type proportions.

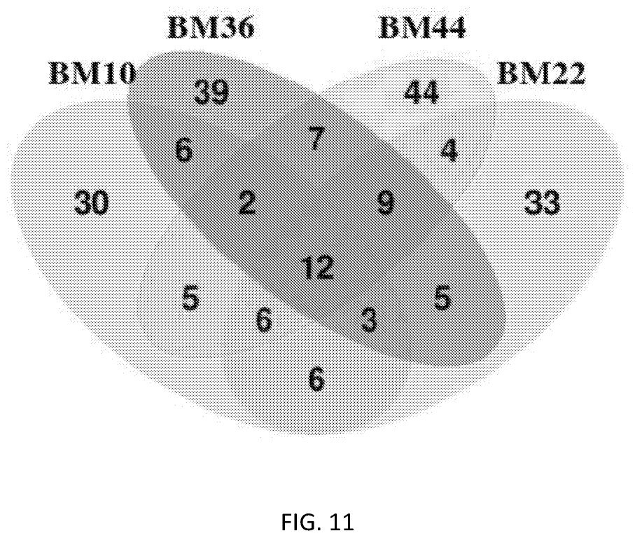

Changes in circRNA expression are correlated with the AD neuropathological quantitative trait: mean number of amyloid plaques. The MSBB dataset includes an additional AD neuropathological quantitative trait: mean number of amyloid beta plaques. 95 circRNAs were identified (Dube, 2019) that were significantly correlated with mean number of plaques, e.g. circHOMER1 (log2 fold change per unit of mean number of plaques: −0.02, p-value: 4.70×10⁻9 in the BM44 subset of the dataset (nPlaqueMean=195). Similarly circRNAs correlated with mean number of plaques were identified in the other cortical regions included in the MSBB dataset (Dube, 2019). Altogether, 12 circRNAs were significantly correlated with mean number of amyloid plaques in all four cortical regions (FIG. 11).

Significantly associated circRNAs explain more of the variation in mean number of amyloid plaques than number of APOE4 alleles or estimated neuronal proportion. It was investigated whether those circRNAs were identified as significantly associated with the mean number of plaques explained more of the variation in the mean number of plaques, compared to the known contributors: number of APOE4 alleles and estimated proportion of neurons (EstNeuron).

Consistent with our results from the other two AD quantitative traits (CDR and Braak score), the top 10 most significant PlaqueMean-associated circRNAs cumulatively explained more of the observed variation in mean number of amyloid plaques across all four cortical regions in the MSBB dataset (Dube, 2019) compared to APOE4 and EstNeuron.

Significantly associated circRNAs explain more of the variation in Braak score than number of APOE4 alleles or estimated neuronal proportion in individuals with ADAD. It was also investigated whether those circRNAs identified as significantly associated with Braak score adjusted ADAD versus AD case status explained more of the variation in Braak score, compared to the known contributors: number of APOE4 alleles and estimated proportion of neurons (EstNeuron) in the individuals with ADAD (nADAD with Braak=17). Consistent with our results for Braak score in the discovery and replication datasets, the top 10 most significant Braak score-adjusted, ADAD-associated circRNAs cumulatively explained more of the observed variation in Braak score (Dube, 2019) compared to APOE4 and EstNeuron.

AD-associated circRNAs improve sensitivity and specificity of logistic models predicting AD case status. Given the robust association between circRNA expression and AD traits, it was investigated if including circRNAs improved the sensitivity and specificity of logistic models predicting AD case status (FIG. 14). Our base, demographic-genetic model included the differential expression covariates (PMI, TIN, AOD, batch, sex, genetic ancestry) as well as number of APOE4 alleles and yielded good predictive value—as measured by area under the receiver operator characteristic curve (AUC)—in both the discovery (n_{control}=13, n_{AD}=83, AUCbase: 0.90) and replication (n_{control}=40, n_{Definite AD=}89, AUCbase: 0.84) datasets. Interestingly, models that only included normalized counts for the top 10 circRNAs most significantly associated with CDR on meta-analysis, yielded better predictive values in both the discovery (AUCcirc: 0.97) dataset and the replication (AUCcirc: 0.88) dataset. Finally, combining the models yielded the best predictive ability in both the discovery (AUCbase+circ: 1.00) and replication (AUCbase+circ: 0.98) datasets. Similar results for the other MSBB cortical regions were observed with the addition of circRNA expression information increasing the predictive ability above the base demographic-genetic model (FIG. 14). These results indicate that cortical circRNA expression is highly predictive of AD case status.

Data and Data Tables not shown here can be found in Dube, U., Del-Aguila, J. L., Li, Z. et al. An atlas of cortical circular RNA expression in Alzheimer disease brains demonstrates clinical and pathological associations. Nat Neurosci 22, 1903-1912 (2019) (Dube, 2019), which is incorporated by reference in its entirety.

### Discussion

Transcriptional regulation underlies the complexity of the human nervous system, and its misregulation can contribute to disease (Lee T. I. et al., Cell 152, 1237-1251 (2013)). Indeed, several studies focused on the linear transcriptome have identified co-expression networks and changes in splicing associated with AD case status. In the present Example, insight into the AD-associated circular transcriptome is provided.

Using two large and independent, brain-derived, RNA-seq datasets, it was established that changes in specific circRNAs are a replicable phenomenon in AD. It was demonstrated that circRNA expression levels are significantly correlated with both neuropathological and clinical measures of AD severity, suggesting an important role in the disease (Table 1). This role is further supported by evidence for changes in circRNA expression early in pre-symptomatic AD. The pathological processes underlying AD follow a well-characterized spatiotemporal progression, which begins decades before substantial symptom onset. Thus, changes in circRNA expression during the pre-symptomatic stage, which are observed as occurring across the cortex in a sequence consistent with the known spatiotemporal progression of AD pathology, may directly contribute to disease rather than being merely correlated. The finding of the present Example that the magnitude of changes in circRNA expression were greater in individuals with the genetically driven ADAD compared with sporadic AD, even after adjusting for neuropathological severity, also argues against AD-associated circRNAs being merely correlated with disease. An important role is further supported by the network analyses, which demonstrate that AD-associated circRNAs co-express with genes known to be part of AD causal pathways.

The present Example identified 164 AD-associated circRNAs on meta-analysis and performed network co-expression and miRNA-binding site prediction analyses to infer biological context and facilitate the interpretation of the association results. For example, circHOMER1, which was significantly associated with all three AD traits, co-expressed with linear genes involved in AD and oxidative phosphorylation, perhaps suggesting a role for this circRNA in brain hypometabolism associated with AD. Brain hypometabolism has also been demonstrated in PSEN1 mutation-driven ADAD and circHOMER1 contains multiple predicted binding sites for miR-651, an miRNA predicted to target PSEN1 and PSEN2. Similarly, circCORO1C was identified as co-expressing with the AD-related genes APP and SNCA, and its sequence was predicted to contain multiple miR-105-binding sites. miR-105 is predicted to target both APP and SNCA, suggesting that the co-expression observed may be mediated through this miRNA. Importantly, if this and other AD-associated circRNAs exert functional effects through miRNA regulation, then subtle changes in circRNA expression may have major impacts on downstream gene expression.

The identification of high-confidence circRNA expression in the present Example is technically limited by the high depth of sequencing and large number of samples required to generate sufficient reads for calling and stringently filtering backsplice junctions. In addition, circRNAs can only be called efficiently in rRNA-depleted RNA-seq datasets, which are currently uncommon. The results of the present Example support the generation of additional AD and control, brain rRNA-depleted, RNA-seq datasets.

Sensitivity analyses demonstrate that most of the circRNA-AD associations are independent of cognate linear mRNA or estimated cell-type proportion changes associated with AD—despite the inherent technical (linear) or biological (cell-type proportion) correlation. Nevertheless, the linear-circular technical correlation limits the interpretation of co-expression modules that include both AD-associated circRNAs and their cognate linear mRNAs. In addition, some AD-associated circRNAs may not be independent of their AD-linear mRNA associations, but, as the biological functions of circRNAs are different, these AD-associated circRNAs may still be pathologically relevant. Finally, the present Example identified some AD-associated circRNA-cognate linear mRNA pairs for which the circRNA appeared to be driving the association with AD. This finding suggests that circRNA analyses can be conducted alongside traditional linear mRNA differential expression analyses to investigate for this possibility in other rRNA-depleted RNA-seq datasets.

In the present Example, circRNA expression was observed to yield strong predictive ability for AD case status, even in the absence of demographic or APOE4 risk factor data. This observation, coupled with the relative stability of circRNAs in biofluids, such as cerebrospinal fluid and plasma (Maass, P. G. et al., J. Mol. Med. 1-11 (2017) doi:10.1007/s00109-017-1582-9) and their enrichment in exosomes, suggests that circRNAs have use as peripheral biomarkers of pre-symptomatic and symptomatic AD and other neurodegenerative diseases.

### Example 2

### Circular RNA as a Non-Invasive (Blood-Based) Biomarker of Alzheimer's Disease

The following example describes the identification of a robust and replicable differential expression (DE) of circular RNAs in brain tissues from neuropathologically-confirmed AD cases and controls. circRNAs are so significantly associated with AD, that a predictive model using the counts of only 10 DE circRNAs in brain tissues provided high specificity and sensitivity (AUC: 0.88). In addition, it was observed that circRNA DE was also significantly associated with both clinical and pathological AD severity. Consistent with this, it was also observed that nominally significant circRNA DE in pre-symptomatic brains showed evidence of AD pathology, but no clinical dementia when compared to control brains and more severe DE in brains from individuals with ADAD. Co-expression of AD-related genes with AD circRNAs were observed through network analysis. Altogether, the present disclosure provides multiple lines of evidence linking changes in circRNA expression to AD.

The pathological processes underlying Alzheimer's Disease (AD) begins decades before cognitive symptom onset. Thus, there exists a lengthy period of time in which disease-modifying or neuroprotective therapies could delay or even prevent AD onset. The Alzheimer's Association has estimated the societal and economic benefits of such therapies to be in the billions of dollars. Currently, research efforts towards identifying disease modifying or neuroprotective therapies for AD have been limited by current methods for identifying at-risk populations for enrollment in clinical trials. One promising approach pioneered by the Dominantly Inherited Alzheimer's Network (DIAN) has been to focus on the rare population of families with pathogenic mutations in known Mendelian genes for AD—autosomal dominant AD (ADAD). Individuals with such mutations are genetically at-risk and develop AD far earlier than individuals with sporadic AD. They also demonstrate more severe neuropathology compared with sporadic AD. Therefore, any therapeutic candidates identified in the rare ADAD population may not be generalizable to the 99% of the AD population with sporadic AD. Additionally, the number of therapies that can be studied in this population is limited by the relatively small number of families with Mendelian AD mutations. To address the growing societal need for AD disease-modifying or neuroprotective therapies, an economically feasible screening method to identify individuals with pre-symptomatic sporadic AD and accurately diagnose and stage symptomatic sporadic AD must be developed.

Current methods to screen for pre-symptomatic AD rely on CSF-based biomarkers or PET based amyloid imaging studies. These methods, while successful in identifying pre-symptomatic AD, require specialized healthcare providers and invasive procedures—a lumbar puncture to collect CSF or the injection of radioactive tracer for PET imaging—and are expensive. Together, the risk and cost of these methods have likely prevented their use for population-based screening to identify pre-symptomatic AD. A more feasible method for screening would be a blood-based test. Such tests are a routine part of medical care with blood commonly collected in primary care settings. Blood contains cell-derived metabolites, nucleic acids, exosomes, and other constituents allowing it to provide a relatively non-invasive readout of cellular health and pathology. In particular, blood nucleic acids have been leveraged for the sensitive and specific detection of infectious diseases like human immunodeficiency virus using a polymerase chain reaction (PCR)-based approach, even in resource-limited settings. Next generation sequencing-based approaches can also be used to measure nucleic acids within blood; however, its current expense is likely prohibitive for use in population screenings. Nevertheless, once panels of nucleic acids that are sensitive and specific for the trait of interest have been identified using sequencing-based approaches, they can be readily assayed by relatively inexpensive PCR-based methods.

Linear cell-free nucleic acids are subject to constant degradation by blood exonucleases. In contrast, circular RNAs (circRNAs) do not have free hydroxyl ends and are relatively stable. circRNAs are formed from back splicing, where the 3′ end of linear RNA are covalently spliced with the 5′ ends, thereby forming a continuous loop. Detection of backsplice junctions in RNA-sequencing data or by PCR has allowed for the profiling of circRNAs in all organ systems. These studies have found that circRNAs are most enriched in the nervous system, and are particularly highly expressed in synapses. Nervous system circRNA expression has been observed to occur independently of host gene expression and to be regulated during development and in response to neuronal excitation. Thus, measurement of circRNA expression has the potential to provide insight into neuronal and synaptic health. Much is still unknown regarding circRNA biology; for example, it was only recently demonstrated that circRNAs can be translated in vivo. Nevertheless, circRNAs detected in the blood have been found to have utility as biomarkers in cancer, stroke, and multiple sclerosis, but have yet to be investigated as a biomarker for AD.

A robust and replicable differential expression (DE) of circular RNAs in brain tissues from neuropathologically-confirmed AD cases and controls was identified. circRNAs are so significantly associated with AD, that a predictive model using the counts of only 10 DE circRNAs in brain tissues provided high specificity and sensitivity (AUC: 0.88). In addition, we observed that circRNA DE was also significantly associated with both clinical and pathological AD severity. Consistent with this, we also observed nominally significant circRNA DE in pre-symptomatic brains—evidence of AD pathology but no clinical dementia—compared to control brains and more severe DE in brains from individuals with ADAD. We have also observed co-expression of AD-related genes with AD circRNAs through network analysis. Altogether, we have multiple lines of evidence linking changes in circRNA expression to AD.

Some of the AD DE circRNAs we identified have been reported to be detected in human plasma samples. Thus, it is suggested that AD pathology-mediated changes in circRNA levels will be detectable in the plasma, even in pre-symptomatic individuals. The present Example investigates plasma circRNAs as a readily assayable and relatively inexpensive blood-based biomarker for the accurate diagnosis of AD and stratification of patients based on disease severity. The results of which provide the development of an economically viable screening test for symptomatic and pre-symptomatic AD.

Blood-based circRNA biomarkers are broadly applicable beyond diagnosis and stratification. In contrast to a recent report of a mass spectrometry-based blood plasma test for AD, blood-based circRNA biomarkers do not rely on the measurement of potential AD targets, e.g. amyloid beta or tau, and thus have the potential to be companion diagnostics. In addition, as we demonstrated in our brain-based preliminary analyses, circRNA expression tracks with both clinical and pathological severity, thereby enabling blood-based circRNA biomarkers to have the potential to be used to monitor disease progression. Finally, blood-based circRNA biomarkers also have potential applications to other diseases with synaptic and nervous system pathology, e.g. Frontotemporal dementia, Parkinsons disease dementia, and Lewy body dementia.

Using the parietal cortex circRNA data (n_{control}=13, n_{AD}=83), we conducted a transcriptome-wide analysis of circRNA DE. Through this analysis, we identified 9 significantly DE circRNAs (e.g. circHOMER1, p-val: 2.8×10−6). More than half of these signals replicated in our analysis of a larger, independent public RNA-seq dataset of inferior frontal gyrus tissues (ncontrol=40, nAD=89), where we identified 118 DE circRNAs (e.g. circHOMER1, p-val: 1.2×10−5; circCORO1c, p-val: 1.2×10−4). Interestingly, in both datasets circRNA expression was more significantly associated with AD neuropathological severity as measured by Braak score (e.g. parietal—circHOMER1, p-val: 1.7×10−7).

Change in DE circRNAs are observed in non-demented individuals with AD pathology (pre-symptomatic cases). We leveraged 18 pre-symptomatic AD cases in the inferior frontal gyrus RNA-seq dataset to assess for changes in circRNA expression early in the AD process. While this small sample size limited power to detect transcriptome-wide DE circRNAs, we observed 24 of the 118 previously mentioned DE circRNAs to be nominally DE between pre-symptomatic AD vs. controls (e.g. circST18, p-val: 1.3×10−4).

circRNAs are predictive of AD case control status. Ten DE circRNAs (circHOMER1, circDOCK1, circKCNN2, circMAN2A1, circST18, circATRNL1, circEXOSC1, circFMN1, circICA1, circRTN4) were used in a logistic regression model to predict AD case status between 40 neuropathologically-confirmed controls and 89 neuropathologically-confirmed, definite AD cases in the inferior frontal gyrus data and a ROC was calculated. The ROC and AUC values are presented below.

circRNAs are presently found to be detectable in human plasma samples. Applicants have found circRNA levels in blood from the circRNAs described above and find that these circRNAs are differentially expressed between AD cases and controls and in the expected direction, which is the same direction observed in the brain studies. The primers used in the blood studies are the same as described above, specifically, SEQ ID NOs:1-20 as divergent primers pairs to specifically amplify the backsplice junction of DE circRNAs

Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All references, patents and patent applications disclosed herein are incorporated by reference with respect to the subject matter for which each is cited, which in some cases may encompass the entirety of the document.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

1. A method for detecting Alzheimer's disease in a subject, the method comprising:
a) measuring the level of expression of one or more circRNA in a biological sample obtained from a subject; and
b) comparing the level of the at least one circRNA with a predetermined reference level, wherein if the level of the at least one circRNA is above or below the respective reference value, the subject is determined to be an asymptomatic individual at risk of developing AD.

2. The method of claim 1, wherein at least one circRNA is circHOMER1.

3. The method of claim 2, wherein measuring circHOMER1 comprising contacting the sample with primer pairs of SEQ ID NOs 1 and 2.

4. The method of claim 1, wherein the one or more circRNAs are selected from circHOMER1, circDOCK1, circKCNN2, circMAN2A1, circICA1, circFNM1, circRTN4, circST18, circATRNL1, and circEXOSC1.

5. A method of monitoring AD disease progression in a subject comprising:
a) obtaining a first biological sample for the subject and at a later time obtaining a second biological sample for the subject;
b) measuring the level of expression of one or more circRNA in the first and second biological samples; and
c) comparing the level of the at least one circRNA from the second biological sample with the circRNA from the first biological sample, wherein if the level of the at least one circRNA from the second sample is above or below the level of the at least one circRNA from the first sample, the subject is determined to have increased disease progression.

6. The method of claim 5, wherein at least one circRNA is circHOMER1.

7. The method of claim 6, wherein measuring circHOMER1 comprising contacting the sample with primer pairs of SEQ ID NOs 1 and 2.

8. The method of claim 5, wherein the one or more circRNAs are selected from circHOMER1, circDOCK1, circKCNN2, circMAN2A1, circICA1, circFNM1, circRTN4, circST18, circATRNL1, and circEXOSC1.

9. The method of claim 8, wherein measuring the one or more circRNAs comprising contacting the biological sample with a primer pair selected from SEQ ID NOs: 1-2, SEQ ID NOs: 3-4, SEQ ID NOs: 5-6, SEQ ID NOs: 7-8, SEQ ID NOs: 9-10, SEQ ID NOs: 11-12, SEQ ID NOs: 13-14, SEQ ID NOs: 16-15, SEQ ID NOs: 17-18, and SEQ ID NOs: 19-20.

10. The method of claim 5, wherein the one or more circRNAs are selected from circHOMER1, circDOCK1, circKCNN2, circMAN2A1, circST18, circATRNL1, circEXOSC1, circICA1, circFMN1, circRTN4, circCDR1-AS, circMAP7, circTTLL7, circFANCL, circEPB41L5, circCORO1C, circDGKI, circKATNAL2, circWDR78, circADGRB3, circPLEKHM3, circERBIN, circPICALM, circRNASEH2B, circPDE4B, circPHC3, circFAT3, circMLIP, circLPAR1, circSLAIN2, circSPHKAP, circYY1AP1, and circDNAJC6.

11. The method of claim 10, wherein the subject is classified with a disease status or disease severity when circDOCK1, circMAN2A1, circST18, circEXOSC1, circRTN4, circCDR1-AS, circMAP7, circTTLL7, circFANCL, circCORO1C, circWDR78, circERBIN, circPICALM, circRNASEH2B, circPHC3, circLPAR1, circSLAIN2, circYY1AP1, and circDNAJC6 are increased relative to a reference value and/or one or more of circHOMER1, circKCNN2, circATRNL1, circICA1, circFMN1, circEPB41L5, circDGKI, circKATNAL2, circPLEKHM3, circPDE4B, circFAT3, circMLIP, circSPHKAP, and circDNAJC6 are decreased relative to a reference value.

12. A method of treating a subject having or suspected of having AD comprising: in a subject comprising:
a) measuring the level of expression of one or more circRNAs in a biological sample obtained from a subject;
b) comparing the level of the at least one circRNA with a predetermined reference level; and
c) administering to the subject a pharmaceutical composition to correct or stabilize the differentially expressed at least one circRNA measured in step a).

13. The method of claim 12, wherein at least one circRNA is circHOMER1.

14. The method of claim 12, wherein the one or more circRNAs are selected from circHOMER1, circDOCK1, circKCNN2, circMAN2A1, circICA1, circFNM1, circRTN4, circST18, circATRNL1, and circEXOSC1.

15. The method of claim 12, wherein the one or more circRNAs are selected from circHOMER1, circDOCK1, circKCNN2, circMAN2A1, circST18, circATRNL1, circEXOSC1, circICA1, circFMN1, circRTN4, circCDR1-AS, circMAP7, circTTLL7, circFANCL, circEPB41L5, circCORO1C, circDGKI, circKATNAL2, circWDR78, circADGRB3, circPLEKHM3, circERBIN, circPICALM, circRNASEH2B, circPDE4B, circPHC3, circFAT3, circMLIP, circLPAR1, circSLAIN2, circSPHKAP, circYY1AP1, and circDNAJC6.

16. The method of claim 12, wherein the pharmaceutical composition comprises a cholinesterase inhibitor, an N-methyl D-aspartate (NMDA) antagonist, an antidepressant, a gamma-secretase inhibitor, a beta-secretase inhibitor, an anti-Aβ antibody, an anti-tau antibody, an antagonist of the serotonin receptor 6, a p38alpha MAPK inhibitor, recombinant granulocyte macrophage colony-stimulating factor, a passive immunotherapy, an active vaccine, a tau protein aggregation inhibitor, an anti-inflammatory agent, a phosphodiesterase 9A inhibitor, a sigma-1 receptor agonist, a kinase inhibitor, a phosphatase activator, a phosphatase inhibitor, an angiotensin receptor blocker, a CB1 and/or CB2 endocannabinoid receptor partial agonist, a β-2 adrenergic receptor agonist, a nicotinic acetylcholine receptor agonist, a 5-HT2A inverse agonist, an alpha-2c adrenergic receptor antagonist, a 5-HT 1A and 1D receptor agonist, a glutaminyl-peptide cyclotransferase inhibitor, a selective inhibitor of APP production, a monoamine oxidase B inhibitor, a glutamate receptor antagonist, an AMPA receptor agonist, a nerve growth factor stimulant, a HMG-CoA reductase inhibitor, a neurotrophic agent, a muscarinic M1 receptor agonist, a GABA receptor modulator, a PPAR-gamma agonist, a microtubule protein modulator, a calcium channel blocker, an antihypertensive agent, a statin, or any combination thereof.

17. The method of claim 12, comprising traditional linear mRNA analyses.

18. The method of claim 12, wherein the biological sample is a biological fluid, blood, brain tissue, CSF, or plasma.
